(19) United States
(12) Patent Application Publication
(10) Pub. No.: US 2026/0098046 A1
(43) Pub. Date: Apr. 9, 2026

(54) RIFABUTIN ANALOGS FOR THE TREATMENT OF DISEASE

(71) Applicant: BIOVERSYS AG, Basel (CH)

(72) Inventors: Kevin ANTRAYGUES, Loos (FR); Marilyne BOUROTTE, Perenchies (FR); Glenn E. DALE, Basel (CH); Olivier DEFERT, Lille (FR); Marc GITZINGER, Basel (CH); Sergio LOCIURO, Basel (CH); Mathieu MAINGOT, Lille (FR); Vincent TREBOSC, Kembs (FR); Nicolas WILLAND, Lille (FR)

(21) Appl. No.: 19/115,111

(22) PCT Filed: Sep. 29, 2023
(86) PCT No.: PCT/EP2023/077054

§ 371 Date: Mar. 25, 2025

(30) Foreign Application Priority Data
Sep. 30, 2022 (EP) 22198959.3

(51) Int. Cl. C07D 498/22; A61K 31/438; A61K 31/444; A61K 31/4545; A61K 31/496; A61K 31/541; A61K 31/55

(52) U.S. Cl. CPC C07D 498/22; A61K 31/438; A61K 31/444; A61K 31/4545; A61K 31/496; A61K 31/541; A61K 31/55

(57) ABSTRACT

The present invention relates to compounds and pharmaceutical compositions comprising the same for the treatment, amelioration and/or prevention of disease. In some embodiments, the disease is a bacterial infection. In some embodiments, the bacterial infection is caused by one or more bacterium belonging to a genus of non-tuberculous Mycobacteria, preferably M. abscessus.

The present invention relates to compounds and pharmaceutical compositions comprising the same for the treatment, amelioration and/or prevention of disease. In some embodiments, the disease is a bacterial infection. In some embodiments, the bacterium belongs to the genus or species Acinetobacter spp., Clostridium spp., Enterococcus spp., Hemophilus spp., Legionella spp., Mycobacterium spp., Neisseria spp., Staphylococcus spp., Streptococcus spp., Listeria monocytogenes, Moraxella catarrhalis, Bacillus spp., Bacteroides spp., Gardnerella vaginalis, Lactobacillus spp., Mobiluncus spp., Helicobacter pylori, Campylobacter jejuni, Chlamydia trachomatis and/or Toxoplasma gondii. In some embodiments the infection is caused by a A. baumannii, and/or S. aureus, and/or a genus of non-tuberculous Mycobacteria (NTM), preferably M. abscessus.

### RELATED ART

Rifamycins such as rifabutin are known antibiotics with activity against a broad spectrum of pathogens such as Clostridium spp., Enterococcus spp., Hemophilus spp., Legionella spp., Mycobacterium spp. (tuberculous and non-tuberculous Mycobacteria), Neisseria spp., Staphylococcus spp., Streptococcus spp., Listeria monocytogenes, Moraxella catarrhalis, Bacillus spp., Bacteroides spp., Gardnerella vaginalis, Lactobacillus spp., Mobiluncus spp., Helicobacter pylori, Campylobacter jejuni, Chlamydia trachomatis and Toxoplasma gondii (Kunin, Clin. Infect. Dis., 1996; Farr and Mandell, Med. Clin. North. Am., 1982; Thornsberry et al., Rev. Infect. Dis., 1983; Hoover et al., Diagn. Microbiol. Infect. Dis., 1993; Kerry et al., J. Antimicrob. Chemother., 1975).

Rifabutin has been recently shown to have potent in vitro and in vivo activity against Mycobacterium abscessus (Aziz et al., Antimicrob. Agents Chemother., 2017; Dick et al., Antimicrob. Agents Chemother., 2020) and Acinetobacter baumannii (Luna et al., Nat. Microbiol., 2020; Trebosc et al., Drug Discov. Today, 26(9), 2021, pp. 2099-2104; Trebosc et al., J. Antimicrob. Chemother., 2020;). C21-modified prodrugs of rifabutin for intravenous administration in A. baumannii infections were described by Antraygues et al., Eur. J. Med. Chem., 238, 2022. A rifamycin-nitroimidazole coupling molecule for the treatment of nontuberculosus mycobacteria was described by Ma et al., (US 2020/0360352). Substituted rifamycin derivatives in which a nitroimidazole, nitrothiazole, or nitrofuran pharmacophore is covaently bound to a rifamycin have been described by Ding et al. (WO 2008/008480). Peek et al. described semi-synthetic hybrid antibiotics formed by linking Kangelmycin A, a rifampicin analog, and a collection of fluoroquinolones (Peek et al., Bioorg. & Med. Chem. Lett., 57, 2021). Occelli et al. described rifamycin derivatives with substitution at the 36-position, and activity of such derivatives against gram positive bacteria and fastidious gram-negative bacteria (WO 94/28002). However, there remains a need for more effective rifamycins for the treatment of bacterial infections such as M. abscessus and A. baumannii infections.

### SUMMARY OF THE INVENTION

In one aspect, the present invention provides a compound of Formula (I) or a pharmaceutically acceptable salt, solvate, hydrate, tautomer, or enantiomer, thereof:

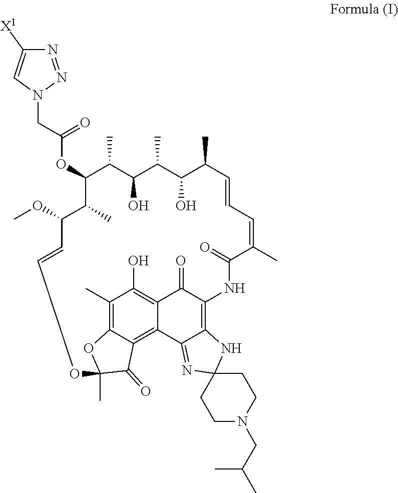

wherein:
X¹ is independently selected from —COOH, —C₁-C₆ alkyl, —C₃-C₈ cycloalkyl, —C₁-C₆ alkylene-(C₃-C₈ cycloalkyl), 5- to 10-membered heterocycloalkyl, —C₁-C₆ alkylene-(5- to 10-membered heterocycloalkyl), —C₆-C₁₀ aryl, —C₁-C₆ alkylene-(C₆-C₁₀ aryl), 5- to 10-membered heteroaryl, and —C₁-C₆ alkylene-(5- to 10-membered heteroaryl);
wherein said alkyl is optionally substituted with one or more R¹;
wherein said cycloalkyl is each independently, at each occurrence, optionally substituted with one or more R²;
wherein said heterocycloalkyl is each independently, at each occurrence, optionally substituted with one or more R³;
wherein said aryl is each independently, at each occurrence, optionally substituted with one or more R⁴;
wherein said heteroaryl is each independently, at each occurrence, optionally substituted with one or more R⁵;
R¹, R², R³, R⁴ and R⁵ are each independently, at each occurrence, selected from —OH, —OC₁-C₆ alkyl, —NR⁶R⁷, —NHSO₂R⁸, —COOH, oxo, —NO₂, phenyl, halogen, and cyano;
R⁶ and R⁷ are each independently, at each occurrence, selected from —H and —C₁-C₆ alkyl, wherein said C₁-C₆ alkyl is optionally substituted with phenyl; and
R⁸ is independently selected from —C₁-C₆ alkyl and phenyl, wherein said phenyl is optionally substituted with —C₁-C₆ alkyl or halogen.

In one aspect, the present invention provides a compound according to Formula (I), or a pharmaceutically acceptable salt, tautomer, solvate or hydrate thereof, or a pharmaceutical composition comprising a compound according to Formula (I), for use as a medicament.

In one aspect, the present invention provides a pharmaceutical composition comprising a compound of Formula (I), or a pharmaceutically acceptable salt thereof, and a pharmaceutically acceptable carrier. In some preferred embodiments, the pharmaceutical composition is effective for treating a bacterial infection. In some preferred embodiments, the bacterial infection is caused by one or more bacterium belonging to the genus Acinetobacter, Staphylococcus, and/or Mycobacteria. In some preferred embodiments, the bacterial infection is caused by one or more bacterium belonging to the species A. baumannii, and/or S. aureus, and/or a genus of non-tuberculous Mycobacteria, preferably M. abscessus. In preferred embodiments, the bacterial infection is caused by one or more bacterium belonging to a genus of non-tuberculous Mycobacteria, preferably M. abscessus. In some embodiments, the infection is caused by one or more bacterium belonging to the genus Acinetobacter and/or Staphylococcus, preferably A. baumannii and/or S. aureus.

In one aspect, the present invention provides a compound of Formula (I) or a pharmaceutically acceptable salt thereof as described herein for use as a medicament. In another aspect, the present invention provides a compound of Formula (I) or a pharmaceutically acceptable salt thereof as described herein for use in a method for treating a bacterial infection. In some preferred embodiments, the bacterial infection is caused by one or more bacterium belonging to the genus Acinetobacter, Staphylococcus, and/or Mycobacteria. In some preferred embodiments, the bacterial infection is caused by one or more bacterium belonging to the species A. baumannii, and/or S. aureus, and/or a genus of non-tuberculous Mycobacteria, preferably M. abscessus. In preferred embodiments, the bacterial infection is caused by one or more bacterium belonging to a genus of non-tuberculous Mycobacteria, preferably M. abscessus. In some embodiments, the infection is caused by one or more bacterium belonging to the genus Acinetobacter and/or Staphylococcus, preferably A. baumannii and/or S. aureus.

In one aspect, the present invention provides a use of a compound of Formula (I) or pharmaceutical composition comprising a compound of Formula (I) in the manufacture of a medicament for treating a bacterial infection. In some preferred embodiments, the bacterial infection is caused by one or more bacterium belonging to the genus Acinetobacter, Staphylococcus, and/or Mycobacteria. In some preferred embodiments, the bacterial infection is caused by one or more bacterium belonging to the species A. baumannii, and/or S. aureus, and/or a genus of non-tuberculous Mycobacteria, preferably M. abscessus. In preferred embodiments, the bacterial infection is caused by one or more bacterium belonging to a genus of non-tuberculous Mycobacteria, preferably M. abscessus. In some embodiments, the infection is caused by one or more bacterium belonging to the genus Acinetobacter and/or Staphylococcus, preferably A. baumannii and/or S. aureus.

In one aspect, the present invention provides a method of treating a bacterial infection in a subject in need thereof, comprising administering to said subject a therapeutically effective amount of a compound of Formula (I) or a pharmaceutically acceptable salt thereof. In some preferred embodiments, the bacterial infection is caused by one or more bacterium belonging to the genus Acinetobacter, Staphylococcus, and/or Mycobacteria. In some preferred embodiments, the bacterial infection is caused by one or more bacterium belonging to the species A. baumannii, and/or S. aureus, and/or a genus of non-tuberculous Mycobacteria, preferably M. abscessus. In preferred embodiments, the bacterial infection is caused by one or more bacterium belonging to a genus of non-tuberculous Mycobacteria, preferably M. abscessus. In some embodiments, the infection is caused by one or more bacterium belonging to the genus Acinetobacter and/or Staphylococcus, preferably A. baumannii and/or S. aureus.

The present invention provides rifabutin analogs that are modified at the C25 position to contain a 2-triazolo acetate ester, wherein said triazole is substituted at the 4-position, and pharmaceutical compositions comprising the same. The inventive compounds exhibit broad antibacterial activity against a wide array of bacterial species, and thus maintain the broad antibacterial activity characteristic of the rifamycin class of antibiotics. Additionally, the inventive compounds unexpectedly showed enhanced antibacterial activity against non-tuberculous Mycobacteria including M. abscessus compared to currently available antibiotics (e.g., rifabutin). Additional features and advantages of the present technology will be apparent to one of skill in the art upon reading the Detailed Description, below.

### DETAILED DESCRIPTION

The present invention provides analogs of rifabutin that are effective in treating bacterial infections, preferably bacterial infections caused by one or more bacterium belonging to a genus of non-tuberculous Mycobacteria, preferably M. abscessus.

### Definitions

Unless defined otherwise, all technical and scientific terms used herein have the same meanings as commonly understood by one of ordinary skill in the art to which this invention belongs.

The articles “a” and “an” are used in this disclosure to refer to one or more than one (i.e., to at least one) of the grammatical object of the article unless the context clearly dictates otherwise. By way of example, “an element” means one element or more than one element.

The term “and/or” is used in this disclosure to mean either “and” or “or” unless indicated otherwise.

The term “optionally substituted” is understood to mean that a given chemical moiety (e.g. an alkyl group) can (but is not required to) be bonded other substituents (e.g. heteroatoms). For instance, an alkyl group that is optionally substituted can be a fully saturated alkyl chain (i.e. a pure hydrocarbon). Alternatively, the same optionally substituted alkyl group can have substituents different from hydrogen. For instance, it can, at any point along the chain be bounded to a halogen atom, a hydroxyl group, or any other substituent described herein. Thus, the term “optionally substituted” means that a given chemical moiety has the potential to contain other functional groups, but does not necessarily have any further functional groups.

The term “alkyl” refers to a straight or branched chain saturated hydrocarbon. C₁-C₆ alkyl groups contain 1 to 6 carbon atoms. Examples of a —C₁-C₆ alkyl group include, but are not limited to, methyl, ethyl, propyl, butyl, pentyl, isopropyl, isobutyl, sec-butyl and tert-butyl, isopentyl and neopentyl.

The terms “alkylene” or “alkylenyl,” as used herein, refer to a straight or branched hydrocarbon chain bi-radical derived from alkyl, as defined herein, wherein one hydrogen of said alkyl is cleaved off generating the second radical of said alkylene. Examples of alkylene are, by way of illustration, —CH₂—, —CH₂—CH₂—, —CH(CH₃)—, —CH₂—CH₂—CH₂—, —CH(CH₃)—CH₂—, or —CH(CH₂CH₃)—.

The term “cycloalkyl” means monocyclic or polycyclic saturated carbon rings containing 3-8 carbon atoms. Examples of cycloalkyl groups include, without limitations, cyclopropyl, cyclobutyl, cyclopentyl, cyclohexyl, cycloheptyl, and cyclooctyl.

The term “aryl” refers to cyclic, aromatic hydrocarbon groups that have 1 to 2 aromatic rings, including monocyclic or bicyclic groups such as phenyl and naphthyl. A C₆-C₁₀ aryl group contains between 6 and 10 carbon atoms; preferably 6 or 10 carbon atoms. When containing two aromatic rings (bicyclic, etc.), the aromatic rings of the aryl group may be fused (e.g., naphthyl). The aryl group may be optionally substituted by one or more substituents, e.g., 1 to 5 substituents, at any point of attachment. Exemplary substituents include, but are not limited to, —H, -halogen, —O—C₁-C₆ alkyl, —C₁-C₆ alkyl, —OH, —NH₂, —NH(C₁-C₆ alkyl), and —N(C₁-C₆ alkyl)₂. In some embodiments, the aryl group can be optionally substituted by a substitutent selected from the group consisting of —OH, —OC₁-C₆ alkyl, —NR⁶R⁷, —NHSO₂R⁸, —COOH, oxo, —NO₂, phenyl, halogen, and cyano. The substituents (e.g., alkyl groups) can themselves be optionally substituted.

Unless otherwise specifically defined, “heteroaryl” means a monovalent monocyclic or bicyclic aromatic radical of 5 to 10 ring atoms containing one or more ring heteroatoms selected from N, S, P, and O, the remaining ring atoms being C. Preferably the heteroatom is selected from N, S, and O, more preferably N and O. The aromatic radical is optionally substituted independently with one or more substituents described herein. Examples include, but are not limited to, furyl, thienyl, pyrrolyl, pyridyl, pyrazolyl, pyrimidinyl, imidazolyl, isoxazolyl, oxazolyl, oxadiazolyl, pyrazinyl, indolyl, quinolyl, isothiazolyl, thiazolyl, thiadiazole, indazole, benzimidazolyl, 1,3-dihydro-2H-benzimidazol-2-one, thieno[3,2-b]thiophene, triazolyl, triazinyl, imidazo[1,2-b]pyrazolyl, furo[2,3-c]pyridinyl, imidazo[1,2-a]pyridinyl, indazolyl, pyrrolo[2,3-c]pyridinyl, pyrrolo[3,2-c]pyridinyl, pyrazolo[3,4-c]pyridinyl, thieno[3,2-c]pyridinyl, thieno[2,3-c]pyridinyl, thieno[2,3-b]pyridinyl, benzothiazolyl, benzofuran, quinolinyl, isoquinolinyl, 1,6-naphthyridinyl, thieno[2,3-b]pyrazinyl, quinazolinyl, tetrazolo[1,5-a]pyridinyl, [1,2,4]triazolo[4,3-a]pyridinyl, pyrrolo[2,3-b]pyridinyl, pyrrolo[3,4-b]pyridinyl, pyrrolo[3,2-b]pyridinyl, imidazo[5,4-b]pyridinyl, pyrrolo[1,2-a]pyrimidinyl, pyridin-2-one, furo[3,2-c]pyridinyl, furo[2,3-c]pyridinyl, benzooxazolyl, benzisoxazolyl, furo[2,3-b]pyridinyl, benzothiophenyl, 1,5-naphthyridinyl, furo[3,2-b]pyridine, [1,2,4]triazolo[1,5-a]pyridinyl, benzo[1,2,3]triazolyl, imidazo[1,2-a]pyrimidinyl, [1,2,4]triazolo[4,3-b]pyridazinyl, benzo[c][1,2,5]thiadiazolyl, benzo[c][1,2,5]oxadiazole, 1,3-dihydro-2H-benzo[d]imidazol-2-one, thiazolo[5,4-d]thiazolyl, imidazo[2,1-b][1,3,4]thiadiazolyl, and thieno[2,3-b]pyrrolyl.

The terms “heterocyclyl” or “heterocycloalkyl” or “heterocycle” refer to monocyclic or polycyclic saturated or partially saturated 5- to 10-membered rings containing carbon and heteroatoms taken from O, N, and S (preferably O and N) and wherein at least one ring does not comprise delocalized π electrons (aromaticity) shared among the ring carbon or heteroatoms. When the heterocycle is a monocyclic heterocycle, said monocyclic heterocyle does not comprise aromaticity. Heterocyclyl rings include, but are not limited to, oxetanyl, azetadinyl, tetrahydrofuranyl, pyrrolidinyl, oxazolinyl, oxazolidinyl, thiazolinyl, thiazolidinyl, pyranyl, thiopyranyl, tetrahydropyranyl, piperidinyl, morpholinyl, thiomorpholinyl, thiomorpholinyl S-oxide, thiomorpholinyl S-dioxide, piperazinyl, azepinyl, oxepinyl, [1,4]diazepane, [1,2]diazepane, decahydro-[1,6]naphthyridine and diazepinyl. In some embodiments, the heterocyclyl group is fully saturated. In some embodiments, the heterocyclyl group is partially saturated.

A heterocyclyl or heterocycloalkyl ring can be fused or bridged, e.g., can be a bicyclic or tricyclic ring. Furthermore, when containing two or more fused rings, the heterocycloalkyl groups herein defined can have an unsaturated or partially saturated ring fused with an aromatic and/or heteroaromatic ring. Exemplary ring systems of such heterocyle-aryl or heterocyle-heteroaryl groups include indolinyl, indolinonyl, dihydrobenzothiophenyl, dihydrobenzofuran, chromanyl, thiochromanyl, tetrahydroquinolinyl, dihydrobenzothiazine, 3,4-dihydro-1H-isoquinolinyl, 2,3-dihydrobenzofuran, 2,3,4,5-tetrahydro-1H-pyrido[4,3-b]indole, 5,6,7,8-tetrahydro-imidazo[1,2-a]pyrazine, and dihydrobenzoxanyl.

A heterocyclyl or heterocycloalkyl ring can also be a spirocyclic heterocycle or spiroheterocycle. As used herein a spirocyclic heterocycle or spiroheterocycle is understood to mean a bicyclic or multicyclic ring system in which at least two rings are connected through a single atom, and wherein at least one of the rings is a heterocycle (e.g., at least one of the rings is furanyl, morpholinyl, or piperadinyl). One or both of the rings in a spiroheterocycle can be can be fused to one or more additional carbocyclic, heterocyclic, aromatic, or heteroaromatic ring to form, e.g., a tricyclic ring system in which two of the rings are connected through a single atom.

As used herein, the term “halo” or “halogen” means fluoro (F), chloro (Cl), bromo (Br), or iodo (I).

The term “oxo” refers to a carbonyl functional group composing a carbon atom double-bonded to an oxygen atom. It can be abbreviated herein as “oxo”, as C(O), or as C═O.

The invention also includes pharmaceutical compositions comprising an effective amount of a disclosed compound and a pharmaceutically acceptable carrier.

Representative “pharmaceutically acceptable salts” include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, hydroiodide, sethionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

The term “stereoisomers” refers to the set of compounds which have the same number and type of atoms and share the same bond connectivity between those atoms, but differ in three-dimensional structure. The term “stereoisomer” refers to any member of this set of compounds.

The term “diastereomers” refers to the set of stereoisomers which cannot be made superimposable by rotation around single bonds. For example, cis- and trans-double bonds, endo- and exo-substitution on bicyclic ring systems, and compounds containing multiple stereogenic centers with different relative configurations are considered to be diastereomers. The term “diastereomer” refers to any member of this set of compounds. In some examples presented, the synthetic route may produce a single diastereomer or a mixture of diastereomers. In some cases these diastereomers were separated and in other cases a wavy bond is used to indicate the structural element where configuration is variable.

The term “enantiomers” refers to a pair of stereoisomers which are non-superimposable mirror images of one another. The term “enantiomer” refers to a single member of this pair of stereoisomers. The term “racemic” refers to a 1:1 mixture of a pair of enantiomers.

The term “tautomers” refers to a set of compounds that have the same number and type of atoms, but differ in bond connectivity and are in equilibrium with one another. A “tautomer” is a single member of this set of compounds. Typically a single tautomer is drawn but it is understood that this single structure is meant to represent all possible tautomers that might exist. Examples include enol-ketone tautomerism. When a ketone is drawn it is understood that both the enol and ketone forms are part of the present disclosure.

The term “solvate” refers to a complex of variable stoichiometry formed by a solute and solvent. Such solvents for the purpose of the invention may not interfere with the biological activity of the solute. Examples of suitable solvents include, but are not limited to, water, MeOH, EtOH, and AcOH. Solvates wherein water is the solvent molecule are typically referred to as “hydrates.” Hydrates include compositions containing stoichiometric amounts of water, as well as compositions containing variable amounts of water.

An “effective amount” when used in connection with a compound is an amount effective for treating or preventing a disease in a subject as described herein.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The term “treating” with regard to a subject, refers to improving at least one symptom of the subject's disorder. Treating includes curing, improving, or at least partially ameliorating the disorder.

The term “disorder” is used in this disclosure to mean, and is used interchangeably with, the terms disease, condition, or illness, unless otherwise indicated.

The term “administer”, “administering”, or “administration” as used in this disclosure refers to either directly administering a disclosed compound or pharmaceutically acceptable salt of the disclosed compound or a pharmaceutical composition comprising the same to a subject, or administering a prodrug derivative or analog of the compound or pharmaceutically acceptable salt of the compound or pharmaceutical composition to the subject, which can form an equivalent amount of active compound within the subject's body.

A “patient” or “subject” is a mammal, e.g., a human, mouse, rat, guinea pig, dog, cat, horse, cow, pig, or non-human primate, such as a monkey, chimpanzee, baboon or rhesus. Preferably the “patient” or “subject” is a human.

### Compounds of the Invention

In one aspect, the present invention provides a compound of Formula (I) or a pharmaceutically acceptable salt, solvate, hydrate, tautomer, or enantiomer, thereof:

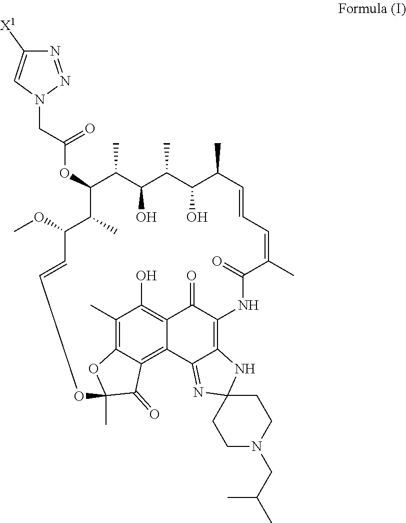

wherein:
X¹ is independently selected from —COOH, —C₁-C₆ alkyl, —C₃-C₈ cycloalkyl, —C₁-C₆ alkylene-(C₃-C₈ cycloalkyl), 5- to 10-membered heterocycloalkyl, —C₁-C₆ alkylene-(5- to 10-membered heterocycloalkyl), —C₆-C₁₀ aryl, —C₁-C₆ alkylene-(C₆-C₁₀ aryl), 5- to 10-membered heteroaryl, and —C₁-C₆ alkylene-(5- to 10-membered heteroaryl);
wherein said alkyl is optionally substituted with one or more R¹;
wherein said cycloalkyl is each independently, at each occurrence, optionally substituted with one or more R²;
wherein said heterocycloalkyl is each independently, at each occurrence, optionally substituted with one or more R³;
wherein said aryl is each independently, at each occurrence, optionally substituted with one or more R⁴;
wherein said heteroaryl is each independently, at each occurrence, optionally substituted with one or more R⁵;
R¹, R², R³, R⁴ and R⁵ are each independently, at each occurrence, selected from —OH, —OC₁-C₆ alkyl, —NR⁶R⁷, —NHSO₂R⁸, —COOH, oxo, —NO₂, phenyl, halogen, and cyano;
R⁶ and R⁷ are each independently, at each occurrence, selected from —H and —C₁-C₆ alkyl, wherein said C₁-C₆ alkyl is optionally substituted with phenyl; and
R⁸ is independently selected from —C₁-C₆ alkyl and phenyl, wherein said phenyl is optionally substituted with —C₁-C₆ alkyl or halogen.

In some embodiments of Formula (I), R¹, R², R³, R⁴ and R⁵ are each independently, at each occurrence, selected from —OH, —OC₁-C₆ alkyl, —NR⁶R⁷, —NHSO₂R⁸, —COOH, oxo, —NO₂, and phenyl;
R⁶ and R⁷ are each independently, at each occurrence, selected from —H and —C₁-C₆ alkyl, wherein said C₁-C₆ alkyl is optionally substituted with phenyl; and
R⁸ is independently selected from —C₁-C₆ alkyl and phenyl, wherein said phenyl is optionally substituted with —C₁-C₆ alkyl or halogen.

In some embodiments of Formula (I), X¹ is independently selected from —COOH, —C₁-C₆ alkyl, —C₃-C₆ cycloalkyl, —C₁-C₆ alkylene-(C₃-C₆ cycloalkyl), 5- to 7-membered heterocycloalkyl, —C₁-C₆ alkylene-(5- to 7-membered heterocycloalkyl), —C₆-C₁₀ aryl, —C₁-C₆ alkylene-(C₆-C₁₀ aryl), 5- to 10-membered heteroaryl, or —C₁-C₆ alkylene-(5- to 10-membered heteroaryl);
wherein said alkyl is optionally substituted with one or more R¹; said cycloalkyl is each independently, at each occurrence, optionally substituted with one or more R²; said heterocycloalkyl is each independently, at each occurrence, optionally substituted with one or more R³; said aryl is each independently, at each occurrence, optionally substituted with one or more R⁴; said 5- to 10-membered heteroaryl is each unsubstituted;
R¹ is independently, at each occurrence, selected from —OH, —NR⁶R⁷, —NHSO₂R⁸, and —COOH;
R² is selected from —NR⁶R⁷;
R³ is oxo; and
R⁴ is independently, at each occurrence, selected from —OC₁-C₆ alkyl, —NR⁶R⁷, —COOH, —NO₂, and phenyl.

In some embodiments of Formula (I), X¹ is independently selected from —COOH, —C₁-C₆ alkyl, —C₃-C₆ cycloalkyl, —C₁-C₆ alkylene-(C₃-C₆ cycloalkyl), 5- to 7-membered heterocycloalkyl, —C₁-C₆ alkylene-(5- to 7-membered heterocycloalkyl), —C₆-C₁₀ aryl, —C₁-C₆ alkylene-(C₆-C₁₀ aryl), 5- to 10-membered heteroaryl, or —C₁-C₆ alkylene-(5- to 10-membered heteroaryl);
wherein said alkyl is optionally substituted with one or more R¹; said cycloalkyl is each independently, at each occurrence, optionally substituted with one or more R²; said heterocycloalkyl is each independently, at each occurrence, optionally substituted with one or more R³; said aryl is each independently, at each occurrence, optionally substituted with one or more R⁴; said 5- to 10-membered heteroaryl is each unsubstituted;
R¹ is independently, at each occurrence, selected from —OH, —NR⁶R⁷, —NHSO₂R⁸, and —COOH;
R² is selected from —NR⁶R⁷;
R³ is oxo; and
R⁴ is independently, at each occurrence, selected from —OC₁-C₆ alkyl, —NR⁶R⁷, —COOH, —NO₂, and phenyl;
R⁶ and R⁷ are each independently, at each occurrence, selected from —H and —C₁-C₆ alkyl, wherein said C₁-C₆ alkyl is optionally substituted with phenyl; and
R⁸ is independently selected from —C₁-C₆ alkyl and phenyl, wherein said phenyl is optionally substituted with —C₁-C₆ alkyl or halogen.

In some embodiments, X¹ is independently selected from —COOH, —C₁-C₅ alkyl, —C₃-C₆ cycloalkyl, —C₁-C₄ alkylene-(C₃-C₆ cycloalkyl), 5- to 7-membered heterocycloalkyl, —C₁-C₄ alkylene-(5- to 7-membered heterocycloalkyl), —C₆-C₁₀ aryl, —C₁-C₄ alkylene-(C₆-C₁₀ aryl), 5- to 6-membered heteroaryl, or —C₁-C₄ alkylene-(5- to 6-membered heteroaryl);
wherein said alkyl is optionally substituted with one to three R¹; said cycloalkyl is each independently, at each occurrence, optionally substituted with one to three R²; said heterocycloalkyl is each independently, at each occurrence, optionally substituted with one to three R³; said aryl is each independently, at each occurrence, optionally substituted with one to three R⁴; said 5- to 6-membered heteroaryl is each unsubstituted;
R¹ is independently, at each occurrence, selected from —OH, —NR⁶R⁷, —NHSO₂R⁸, and —COOH;
R² is —NH₂;
R³ is oxo;
R⁴ is independently, at each occurrence, selected from —OC₁-C₂ alkyl, —NH₂, —NH(C₁-C₂alkyl), —N(C₁-C₂ alkyl)₂, —COOH, —NO₂, and phenyl;
R⁶ and R⁷ are independently, at each occurrence, selected from —H, —CH₃, and —CH₂—C₆H₅; and
R⁴ is independently, at each occurrence, selected from —CH₃ and phenyl, wherein said phenyl is optionally substituted with one or more —CH₃ or halogen.

In some embodiments, X¹ is independently selected from —COOH, —C₁-C₅ alkyl, —C₃-C₆ cycloalkyl, —C₁-C₂ alkylene-(C₃-C₆ cycloalkyl), 5- to 7-membered heterocycloalkyl, —C₁-C₂ alkylene-(5- to 7-membered heterocycloalkyl), —C₆-C₁₀ aryl, —C₁-C₃ alkylene-(C₆-C₁₀ aryl), and 5- to 6-membered heteroaryl;
wherein said alkyl is optionally substituted with one or two R¹, preferably with exactly one R¹; said cycloalkyl is each independently, at each occurrence, optionally substituted with one or two R², preferably with exactly one R²; said heterocycloalkyl is each independently, at each occurrence, optionally substituted with one or two R³, preferably with two R³; said aryl is each independently, at each occurrence, optionally substituted with one or two R⁴, preferably with exactly one R⁴; said 5- to 6-membered heteroaryl is unsubstituted;
R¹ is independently, at each occurrence, selected from —OH, —NH₂, —N(CH₃)(CH₂C₆H₅), —NHSO₂R⁸, and —COOH;
R² is —NH₂;
R³ is oxo;
R⁴ is independently, at each occurrence, selected from —OCH₃, —NH₂, —COOH, —NO₂, and phenyl; and
R⁸ is independently, at each occurrence, selected from —CH₃ and phenyl, wherein said phenyl is optionally substituted with one or more —CH₃ or halogen.

In some embodiments of Formula (I), X¹ is independently selected from —C₁-C₅ alkyl, —C₅-C₆ cycloalkyl, —C₁-C₂ alkylene-(C₅-C₆ cycloalkyl), 5- to 6-membered heterocycloalkyl, —C₁-C₂ alkylene-(5- to 6-membered heterocycloalkyl), —C₆-C₁₀ aryl, —C₁-C₆ alkylene-(C₆-C₁₀ aryl), and 5- to 6-membered heteroaryl;
wherein said alkyl is optionally substituted with one to three R¹, preferably with exactly one R¹; said cycloalkyl is each unsubstituted; said heterocycloalkyl is each independently, at each occurrence, optionally substituted with one or two R³; said aryl is each independently, at each occurrence, optionally substituted with one to three R⁴, preferably with exactly one R⁴;
R¹ is independently, at each occurrence, selected from —NR⁶R⁷ and —NHSO₂R⁸;
R³ is oxo;
R⁴ is independently, at each occurrence, selected from phenyl, —OC₁-C₆ alkyl, NH₂, and —NO₂;
R⁶ and R⁷ are independently, at each occurrence, selected from —H, —CH₃, and —CH₂—C₆H₅; and
R⁸ is —CH₃ or phenyl, wherein said phenyl is optionally substituted with one or more —CH₃ or —C₁.

In some embodiments of Formula (I), X¹ is independently selected from —C₁-C₅ alkyl, —C₅-C₆ cycloalkyl, —C₁-C₂ alkylene-(C₅-C₆ cycloalkyl), 5- to 6-membered heterocycloalkyl, —C₁-C₂ alkylene-(5- to 6-membered heterocycloalkyl), —C₆-C₁₀ aryl, —C₁-C₆ alkylene-(C₆-C₁₀ aryl), and 5- to 6-membered heteroaryl;
wherein said alkyl is optionally substituted with one to three R¹, preferably with exactly one R¹; said cycloalkyl is each unsubstituted; said heterocycloalkyl is each independently, at each occurrence, optionally substituted with one or two R; said aryl is each independently, at each occurrence, optionally substituted with one to three R⁴, preferably with exactly one R⁴;
R¹ is independently, at each occurrence, selected from —NR⁶R⁷ and —NHSO₂R⁸;
R³ is oxo;
R⁴ is independently, at each occurrence, selected from —OC₁-C₆ alkyl, NH₂, and —NO₂;
R⁶ and R⁷ are independently, at each occurrence, selected from —H, —CH₃, and —CH₂—C₆H₅; and
R⁸ is —CH₃ or phenyl, wherein said phenyl is optionally substituted with one or more —CH₃ or —Cl.

In some embodiments, X¹ is independently selected from —C₁-C₃ alkyl, cyclohexyl, —C₁-C₂ alkylene-(cyclohexyl), 5- to 6-membered heterocycloalkyl, —C₁-C₂ alkylene-(5- to 6-membered heterocycloalkyl), —C₆-C₁₀ aryl, —C₁-C₃ alkylene-(C₆-C₁₀ aryl), thiophenyl, and pyridinyl;
wherein said alkyl is optionally substituted with one to three R¹, preferably with exactly one R¹; said cyclohexyl is unsubstituted; said heterocycloalkyl is substituted with one or two oxo; said aryl is each independently, at each occurrence, optionally substituted with one to three R⁴, preferably with exactly one R⁴; said pyridinyl is unsubstituted;
R¹ is independently, at each occurrence, selected from —NH₂, —N(CH₃)(CH₂C₆H₅), and —NHSO₂R⁸.
R⁴ is independently, at each occurrence, selected from —OC₁-C₂ alkyl, —NH₂, and —NO₂;
R⁸ is —CH₃ or phenyl, wherein said phenyl is optionally substituted with one or more —CH₃ or —Cl.

In some embodiments, X¹ is independently selected from —C₁-C₃ alkyl, cyclohexyl, —C₁-C₂ alkylene-(cyclohexyl), 5- to 6-membered heterocycloalkyl, —C₁-C₂ alkylene-(5- to 6-membered heterocycloalkyl), —C₆-C₁₀ aryl, —C₁-C₃ alkylene-(C₆-C₁₀ aryl), thiophenyl, and pyridinyl;
wherein said alkyl is optionally substituted with one to three R¹, preferably with exactly one R¹; said cyclohexyl is unsubstituted; said heterocycloalkyl is substituted with one or two oxo; said aryl is each independently, at each occurrence, optionally substituted with one to three R⁴, preferably with exactly one R⁴; said thiophenyl and pyridinyl are unsubstituted;
R¹ is independently, at each occurrence, selected from —NH₂, —N(CH₃)(CH₂C₆H₅), and —NHSO₂R⁸.
R⁴ is independently, at each occurrence, selected from —OC₁-C₂ alkyl, —NH₂, and —NO₂;
R⁸ is —CH₃ or phenyl, wherein said phenyl is optionally substituted with one or more —CH₃ or —Cl.

In some embodiments, X¹ is independently selected from —C₁-C₃ alkyl, cyclohexyl, —C₁ alkylene-(cyclohexyl), 4-thiomorpholine 1,1-dioxide, —C₁-C₂ alkylene-(4-thiomorpholine 1,1-dioxide), —C₆-C₁₀ aryl, —C₁-C₃ alkylene-(C₆-C₁₀ aryl), 2-thiophenyl, and 2-pyridinyl;
wherein said alkyl is optionally substituted with one or two R¹, preferably with exactly one R¹; said cyclohexyl is unsubstituted; said aryl is unsubstituted or substituted with one or two, preferably with exactly one —OCH₃, —NH₂, or —NO₂; said 2-thiophenyl and 2-pyridinyl are unsubstituted;
R¹ is independently, at each occurrence, selected from —NH₂, —N(CH₃)(CH₂C₆H₅), and —NHSO₂R⁸; and
R⁸ is —CH₃ or phenyl, wherein said phenyl is optionally substituted at the 4-position with —CH₃ or —Cl.

In some embodiments of Formula (I), X¹ is independently selected from —C₁-C₃ alkyl, cyclohexyl, —C₁ alkylene-(cyclohexyl), phenyl, —C₁-C₃ alkylene-(phenyl), and 2-pyridinyl;
wherein said alkyl is optionally substituted with one or two R¹, preferably with exactly one R¹; said cyclohexyl is each unsubstituted; said phenyl each is unsubstituted or substituted with one or two, preferably with exactly one —OCH₃ or —NO₂; said 2-pyridinyl is unsubstituted;
R¹ is independently, at each occurrence, selected from —N(CH₃)(CH₂C₆H₅), and —NHSO₂R⁸; and
R⁸ is phenyl, wherein said phenyl is optionally substituted at the 4-position with —CH₃ or —C₁.

In some embodiments of Formula (I), X¹ is —COOH.

In some embodiments of Formula (I), X¹ is —C₁-C₆ alkyl, wherein said alkyl is optionally substituted with one or more R¹; and wherein R¹ is independently, at each occurrence, selected from —OH, —OC₁-C₆ alkyl, —NR⁶R⁷, —NHSO₂R⁸, —COOH, oxo, —NO₂, phenyl, halogen, and cyano.

In some embodiments, X¹ is —C₁-C₆ alkyl, wherein said alkyl is optionally substituted with one or more R¹; and R¹ is independently, at each occurrence, selected from —OH, —OC₁-C₆ alkyl, —NR⁶R⁷, —NHSO₂R⁸, —COOH, oxo, —NO₂, and phenyl.

In some embodiments, X¹ is —C₁-C₆ alkyl, wherein said alkyl is optionally substituted with one or more R¹; and R¹ is independently, at each occurrence, selected from —OH, —NR⁶R⁷, —NHSO₂R⁸, and —COOH.

In some embodiments, X¹ is —C₁-C₅ alkyl, wherein said alkyl is optionally substituted with one to three R¹; and R¹ is independently, at each occurrence, selected from —OH, —NR⁶R⁷, —NHSO₂R⁸, and —COOH; wherein R⁶ and R⁷ are independently, at each occurrence, selected from —H, —CH₃, and —CH₂—C₆H₅; and wherein R⁸ is independently, at each occurrence, selected from —CH₃ and phenyl, wherein said phenyl is optionally substituted with one or more —CH₃ or halogen.

In some embodiments, X¹ is —C₁-C₅ alkyl, wherein said alkyl is optionally substituted with one or two R¹, preferably with exactly one R¹; and R¹ is independently, at each occurrence, selected from —OH, —NH₂, —N(CH₃)(CH₂C₆H₅), —NHSO₂R⁸, and —COOH; and wherein R⁸ is independently, at each occurrence, selected from —CH₃ and phenyl, wherein said phenyl is optionally substituted with one or more —CH₃ or halogen.

In some embodiments, X¹ is —C₁-C₅ alkyl, wherein said alkyl is optionally substituted with one to three R¹, preferably with exactly one R¹; and R¹ is independently, at each occurrence, selected from —NR⁶R⁷ and —NHSO₂R⁸; wherein R⁶ and R⁷ are independently, at each occurrence, selected from —H, —CH₃, and —CH₂—C₆H₅; and wherein R⁸ is —CH₃ or phenyl, wherein said phenyl is optionally substituted with one or more —CH₃ or —C₁.

In some embodiments, X¹ is —C₁-C₃ alkyl, wherein said alkyl is unsubstituted.

In some embodiments, X¹ is —C₁-C₃ alkyl, wherein said alkyl is optionally substituted with one to three R¹, preferably with exactly one R¹; and R¹ is independently, at each occurrence, selected from —NH₂, —N(CH₃)(CH₂C₆H₅), and —NHSO₂R⁸; wherein R⁸ is —CH₃ or phenyl, wherein said phenyl is optionally substituted with one or more —CH₃ or —C₁.

In some embodiments, X¹ is —C₁-C₃ alkyl, wherein said alkyl is optionally substituted with one or two R¹, preferably with exactly one R¹; and R¹ is independently, at each occurrence, selected from —NH₂, —N(CH₃)(CH₂C₆H₅), and —NHSO₂R⁸; wherein R⁸ is —CH₃ or phenyl, wherein said phenyl is optionally substituted at the 4-position with —CH₃ or —Cl. In some embodiments, X¹ is —C₁-C₃ alkyl, wherein said alkyl is optionally substituted with one to three R¹, preferably with exactly one R¹; and R¹ is independently, at each occurrence, selected from —NH₂, and —N(CH₃)(CH₂C₆H₅).

In some embodiments, X¹ is —C₁ alkyl, wherein said alkyl is optionally substituted with one to three R¹, preferably with exactly one R¹; and R¹ is —NHSO₂R⁸; wherein R⁸ is —CH₃ or phenyl, wherein said phenyl is optionally substituted at the 4-position with —CH₃ or —C₁.

In some embodiments, X¹ is —C₁ alkyl wherein said alkyl is optionally substituted with one to three R¹, preferably with exactly one R¹; and R¹ is —NHSO₂R⁸; wherein R⁸ is phenyl, wherein said phenyl is substituted at the 4-position with —CH₃ or —C₁.

In some embodiments, X¹ is —C₁ alkyl wherein said alkyl is optionally substituted with one to three R¹, preferably with exactly one R¹; and R¹ is —NHSO₂R⁸; wherein R⁸ is phenyl, wherein said phenyl is unsubstituted.

In some embodiments of Formula (I), X¹ is —C₃-C₈ cycloalkyl or —C₁-C₆ alkylene-(C₃-C₈ cycloalkyl), wherein said cycloalkyl is each independently, at each occurrence, optionally substituted with one or more R²; and wherein R² is selected from —OH, —OC₁-C₆ alkyl, —NR⁶R⁷, —NHSO₂R⁸, —COOH, oxo, —NO₂, phenyl, halogen, and cyano.

In some embodiments, X¹ is —C₃-C₆ cycloalkyl or —C₁-C₆ alkylene-(C₃-C₆ cycloalkyl), wherein said cycloalkyl is each independently, at each occurrence, optionally substituted with one or more R²; and wherein R² is selected from —OH, —OC₁-C₆ alkyl, —NR⁶R⁷, —NHSO₂R⁸, —COOH, oxo, —NO₂, and phenyl.

In some embodiments, X¹ is —C₃-C₆ cycloalkyl or —C₁-C₆ alkylene-(C₃-C₆ cycloalkyl), wherein said cycloalkyl is each independently, at each occurrence, optionally substituted with one or more R²; and wherein R² is selected from —NR⁶R⁷.

In some embodiments, X¹ is —C₃-C₆ cycloalkyl or —C₁-C₄ alkylene-(C₃-C₆ cycloalkyl), wherein said cycloalkyl is each independently, at each occurrence, optionally substituted with one to three R²; and wherein R² is —NH₂.

In some embodiments, X¹ is —C₃-C₆ cycloalkyl or —C₁-C₂ alkylene-(C₃-C₆ cycloalkyl), wherein said cycloalkyl is each independently, at each occurrence, optionally substituted with one or two R², preferably with exactly one R²; and wherein R² is —NH₂.

In some embodiments, X¹ is —C₅-C₆ cycloalkyl or —C₁-C₂ alkylene-(C₅-C₆ cycloalkyl), wherein said cycloalkyl is unsubstituted.

In some embodiments, X¹ is cyclohexyl or —C₁-C₂ alkylene-(cyclohexyl), wherein said cyclohexyl is unsubstituted.

In some embodiments, X¹ is cyclohexyl or —C₁ alkylene-(cyclohexyl), wherein said cyclohexyl is unsubstituted.

In some embodiments of Formula (I), X¹ is 5- to 10-membered heterocycloalkyl or —C₁-C₆ alkylene-(5- to 10-membered heterocycloalkyl), wherein said heterocycloalkyl is each independently, at each occurrence, optionally substituted with one or more R³; and wherein R³ is selected from —OH, —OC₁-C₆ alkyl, —NR⁶R⁷, —NHSO₂R⁸, —COOH, oxo, —NO₂, phenyl, halogen, and cyano. In some embodiments, said heterocycloalkyl is saturated.

In some embodiments, X¹ is 5- to 10-membered heterocycloalkyl or —C₁-C₆ alkylene-(5- to 10-membered heterocycloalkyl), wherein said heterocycloalkyl is each independently, at each occurrence, optionally substituted with one or more R³; and wherein R³ is selected from —OH, —OC₁-C₆ alkyl, —NR⁶R⁷, —NHSO₂R⁸, —COOH, oxo, —NO₂, and phenyl. In some embodiments, said heterocycloalkyl is saturated.

In some embodiments, X¹ is 5- to 7-membered heterocycloalkyl or —C₁-C₆ alkylene-(5- to 7-membered heterocycloalkyl), wherein said heterocycloalkyl is each independently, at each occurrence, optionally substituted with one or more R³; and wherein R³ is oxo.

In some embodiments, X¹ is 5- to 7-membered heterocycloalkyl or —C₁-C₄ alkylene-(5- to 7-membered heterocycloalkyl), wherein said heterocycloalkyl is each independently, at each occurrence, optionally substituted with one to three R³; and wherein R³ is oxo.

In some embodiments, X¹ is 5- to 7-membered heterocycloalkyl or —C₁-C₂ alkylene-(5- to 7-membered heterocycloalkyl), wherein said heterocycloalkyl is each independently, at each occurrence, optionally substituted with one or two R³, preferably with two R³; and wherein R³ is oxo.

In some embodiments, X¹ is 5- to 6-membered heterocycloalkyl or —C₁-C₂ alkylene-(5- to 6-membered heterocycloalkyl), wherein said heterocycloalkyl is each independently, at each occurrence, optionally substituted with one or two R³; and wherein R³ is oxo.

In some embodiments, X¹ is 5- to 6-membered heterocycloalkyl or —C₁-C₂ alkylene-(5- to 6-membered heterocycloalkyl), wherein said heterocycloalkyl is substituted with one or two oxo.

In some embodiments, X¹ is 4-thiomorpholine 1,1-dioxide or —C₁-C₂ alkylene-(4-thiomorpholine 1,1-dioxide).

In some embodiments, X¹ is —C₁-C₂ alkylene-(4-thiomorpholine 1,1-dioxide).

In some embodiments, X¹ is —C₁ alkylene-(4-thiomorpholine 1,1-dioxide).

In some embodiments, X¹ is azepane.

In some embodiments, X¹ is piperidine.

In some embodiments, X¹ is a spiroheterocyle, preferably a six- to nine-membered spiroheterocycle.

In some embodiments of Formula (I), X¹ is —C₆-C₁₀ aryl or —C₁-C₆ alkylene-(C₆-C₁₀ aryl), wherein said aryl is each independently, at each occurrence, optionally substituted with one or more R⁴; and wherein R⁴ is independently, at each occurrence, selected from —OH, —OC₁-C₆ alkyl, —NR⁶R⁷, —NHSO₂R⁸, —COOH, oxo, —NO₂, phenyl, halogen, and cyano.

In some embodiments, X¹ is —C₆-C₁₀ aryl or —C₁-C₆ alkylene-(C₆-C₁₀ aryl), wherein said aryl is each independently, at each occurrence, optionally substituted with one or more R⁴; and wherein R⁴ is selected from —OH, —OC₁-C₆ alkyl, —NR⁶R⁷, —NHSO₂R⁸, —COOH, oxo, —NO₂, and phenyl.

In some embodiments, X¹ is —C₆-C₁₀ aryl or —C₁-C₆ alkylene-(C₆-C₁₀ aryl), wherein said aryl is each independently, at each occurrence, optionally substituted with one or more R⁴; and wherein R⁴ is selected from —OC₁-C₆ alkyl, —NR⁶R⁷, —COOH, —NO₂, and phenyl.

In some embodiments, X¹ is —C₆-C₁₀ aryl or —C₁-C₄ alkylene-(C₆-C₁₀ aryl), wherein said aryl is each independently, at each occurrence, optionally substituted with one to three R⁴; and wherein R⁴ is selected from —OC₁-C₂ alkyl, —NH₂, —NH(C₁-C₂ alkyl), —N(C₁-C₂ alkyl)₂, —COOH, —NO₂, and phenyl.

In some embodiments, X¹ is —C₆-C₁₀ aryl or —C₁-C₃ alkylene-(C₆-C₁₀ aryl), wherein said aryl is each independently, at each occurrence, optionally substituted with one or two R⁴, preferably with exactly one R⁴; and wherein R⁴ is selected from —OC₁-C₂ alkyl, —NH₂, —NH(C₁-C₂ alkyl), —N(C₁-C₂ alkyl)₂, —COOH, —NO₂, and phenyl.

In some embodiments, X¹ is —C₆-C₁₀ aryl or —C₁-C₃ alkylene-(C₆-C₁₀ aryl), wherein said aryl is each independently, at each occurrence, optionally substituted with one or two R⁴, preferably with exactly one R⁴; and wherein R⁴ is selected from —OCH₃, —NH₂, —COOH, —NO₂, and phenyl.

In some embodiments, X¹ is —C₆-C₁₀ aryl, wherein said aryl is each independently, at each occurrence, optionally substituted with one or two R⁴, preferably with exactly one R⁴; and wherein R⁴ is selected from —OCH₃, —NH₂, —COOH, —NO₂, and phenyl.

In some embodiments, X¹ is —C₁-C₃ alkylene-(C₆-C₁₀ aryl); and wherein said C₆-C₁₀ aryl is unsubstituted.

In some embodiments, X¹ is —C₁-C₃ alkylene-(phenyl); and wherein said phenyl is unsubstituted.

In some embodiments, X¹ is —C₆-C₁₀ aryl or —C₁-C₆ alkylene-(C₆-C₁₀ aryl), wherein said aryl is each independently, at each occurrence, optionally substituted with one to three R⁴, preferably with exactly one R⁴; and wherein R⁴ is independently, at each occurrence, selected from phenyl, —OC₁-C₆ alkyl, —NH₂, and —NO₂.

In some embodiments, X¹ is —C₆-C₁₀ aryl or —C₁-C₆ alkylene-(C₆-C₁₀ aryl), wherein said aryl is each independently, at each occurrence, optionally substituted with one to three R⁴, preferably with exactly one R⁴; and wherein R⁴ is independently, at each occurrence, selected from —OC₁-C₆ alkyl, —NH₂, and —NO₂.

In some embodiments, X¹ is —C₆-C₁₀ aryl or —C₁-C₆ alkylene-(C₆-C₁₀ aryl), wherein said aryl is each independently, at each occurrence, optionally substituted with one to three R⁴, preferably with exactly one R⁴; and wherein R⁴ is independently, at each occurrence, selected from —OC₁-C₂ alkyl, —NH₂, and —NO₂.

In some embodiments, X¹ is —C₆-C₁₀ aryl or —C₁-C₃ alkylene-(C₆-C₁₀ aryl), wherein said aryl is each independently, at each occurrence, optionally substituted with one to three R⁴, preferably with exactly one R⁴; and wherein R⁴ is independently, at each occurrence, selected from —OC₁-C₂ alkyl, —NH₂, and —NO₂.

In some embodiments, X¹ is —C₆-C₁₀ aryl or —C₁-C₃ alkylene-(C₆-C₁₀ aryl); and wherein said aryl is unsubstituted or substituted with one or two, preferably with exactly one —OC₁-C₂ alkyl, —NH₂, or —NO₂.

In some embodiments, X¹ is —C₆-C₁₀ aryl or —C₁-C₃ alkylene-(C₆-C₁₀ aryl); and wherein said aryl is unsubstituted or substituted with one or two, preferably with exactly one —OCH₃, —NH₂, or —NO₂.

In some embodiments, X¹ is phenyl or —C₁-C₃ alkylene-(phenyl), preferably —C₂-C₃ alkylene-(phenyl), wherein said phenyl is unsubstituted.

In some embodiments, X¹ is phenyl or naphthyl, wherein said phenyl or naphthyl is substituted with one or two, preferably with exactly one —OCH₃.

In some embodiments, X¹ is phenyl, wherein said phenyl is substituted with one or two, preferably with exactly one —NH₂.

In some embodiments, X¹ is phenyl, wherein said phenyl is substituted with one or two, preferably with exactly one —NO₂.

In some embodiments of Formula (I), X¹ is 5- to 10-membered heteroaryl or —C₁-C₆ alkylene-(5- to 10-membered heteroaryl); wherein said heteroaryl is independently, at each occurrence, optionally substituted with one or more R⁵; and wherein R⁵ is selected from —OH, —OC₁-C₆ alkyl, —NR⁶R⁷, —NHSO₂R⁸, —COOH, oxo, —NO₂, phenyl, halogen, and cyano.

In some embodiments, X¹ is 5- to 10-membered heteroaryl or —C₁-C₆ alkylene-(5- to 10-membered heteroaryl), wherein said heteroaryl is independently, at each occurrence, optionally substituted with one or more R⁵; and wherein R⁵ is selected from —OH, —OC₁-C₆ alkyl, —NR⁶R⁷, —NHSO₂R⁸, —COOH, oxo, —NO₂, and phenyl.

In some embodiments, X¹ is 5- to 10-membered heteroaryl or —C₁-C₆ alkylene-(5- to 10-membered heteroaryl), wherein said 5- to 10-membered heteroaryl is each unsubstituted.

In some embodiments, X¹ is 5- to 6-membered heteroaryl or —C₁-C₄ alkylene-(5- to 6-membered heteroaryl), wherein said 5- to 6-membered heteroaryl is each unsubstituted.

In some embodiments, X¹ is 5- to 6-membered heteroaryl, wherein said 5- to 6-membered heteroaryl is unsubstituted.

In some embodiments, X¹ is pyridinyl or thiophenyl.

In some embodiments, X¹ is pyridinyl or thiophenyl, wherein said pyridinyl and thiophenyl are unsubstituted.

In some embodiments, X¹ is 2-pyridinyl or 2-thiophenyl, wherein said 2-pyridinyl and 2-thiophenyl are unsubstituted.

In some embodiments, X¹ is thiophenyl.

In some embodiments, X¹ is thiophenyl, wherein said thiophenyl is unsubstituted.

In some embodiments, X¹ is 2-thiophenyl, wherein said 2-thiophenyl is unsubstituted.

In some embodiments, X¹ is pyridinyl.

In some embodiments, X¹ is pyridinyl, wherein said pyridinyl is unsubstituted.

In some embodiments, X¹ is 2-pyridinyl, wherein said 2-pyridinyl is unsubstituted.

In one or more embodiments, X¹ of a compound of Formula (I) can form one of the structures selected from Table 1, below:

TABLE 1
Exemplary Embodiments of X¹

| Embodiment | Structure |
|---|---|
| 1 | 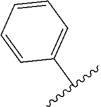 [SMILES: *c1ccccc1] |
| 2 | 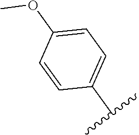 [SMILES: *c1ccc(OC)cc1] |
| 3 | 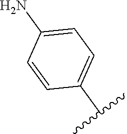 [SMILES: *c1ccc(N)cc1] |
| 4 | 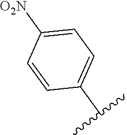 [SMILES: *c1ccc([N+](=O)[O-])cc1] |
| 5 | 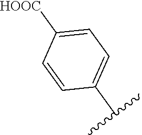 [SMILES: *c1ccc(C(=O)O)cc1] |
| 6 | 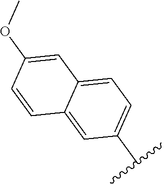 [SMILES: *c1ccc2cc(OC)ccc2c1] |
| 7 | 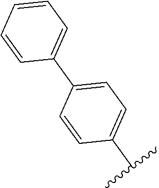 [SMILES: *c1ccc(-c2ccccc2)cc1] |
| 8 | 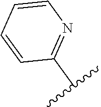 [SMILES: *c1ccccn1] |
| 9 | 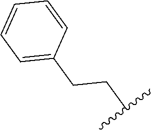 [SMILES: *CCc1ccccc1] |
| 10 | 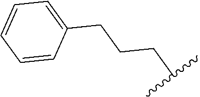 [SMILES: *CCCc1ccccc1] |
| 11 | 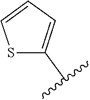 [SMILES: *c1cccs1] |
| 12 | 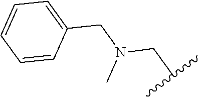 [SMILES: *CN(C)Cc1ccccc1] |
| 13 | 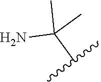 [SMILES: *C(C)(C)N] |
| 14 | 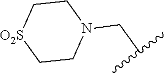 [SMILES: *CN1CCS(=O)(=O)CC1] |
| 15 | 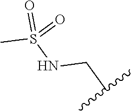 [SMILES: *CNS(C)(=O)=O] |
| 16 | 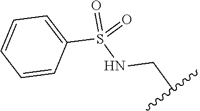 [SMILES: *CNS(=O)(=O)c1ccccc1] |
| 17 | 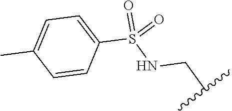 [SMILES: *CNS(=O)(=O)c1ccc(C)cc1] |
| 18 | 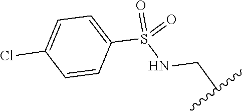 [SMILES: *CNS(=O)(=O)c1ccc(Cl)cc1] |
| 19 | 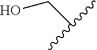 [SMILES: *CO] |
| 20 | 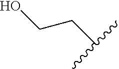 [SMILES: *CCO] |
| 21 | 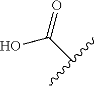 [SMILES: *C(=O)O] |
| 22 | 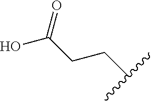 [SMILES: *CCC(=O)O] |
| 23 | 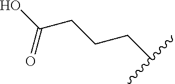 [SMILES: *CCCC(=O)O] |
| 24 | 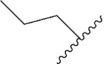 [SMILES: *CCC] |
| 25 | 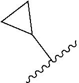 [SMILES: *C1CC1] |
| 26 | 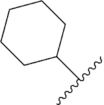 [SMILES: *C1CCCCC1] |
| 27 | 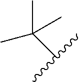 [SMILES: *C(C)(C)C] |
| 28 | 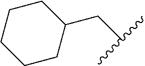 [SMILES: *CC1CCCCC1] |
| 29 | 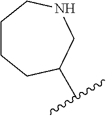 [SMILES: *C1CCCCNC1] |
| 30 | 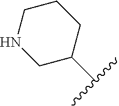 [SMILES: *C1CCCNC1] |
| 31 | 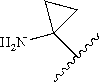 [SMILES: *C1(N)CC1] |
| 32 | 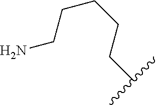 [SMILES: *CCCCCN] |
| 33 | 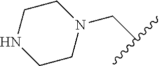 [SMILES: *CN1CCNCC1] |
| 34 | 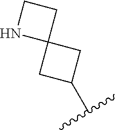 [SMILES: *C1CC2(CCN2)C1] |

In one or more embodiments of any of the above aspects, the compound of Formula (I) is selected from a compound of Table 2, below:

TABLE 2
Exemplary Compounds of the Invention

| Cpd | Structure |
|---|---|
| 1 | 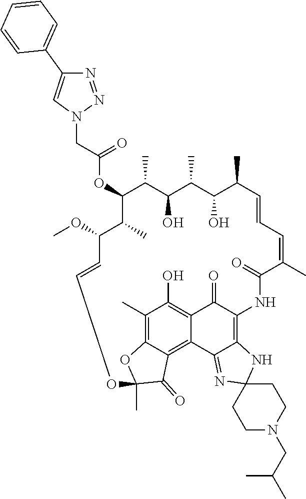 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(-c3ccccc3)nn2)[C@@H]1C)C4=O] |
| 2 | 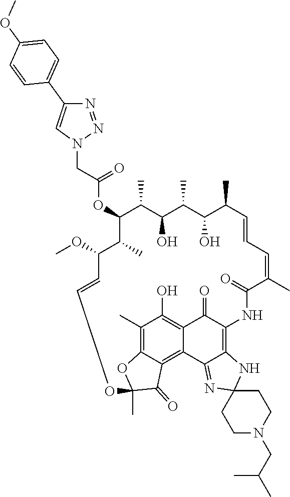 [SMILES: COc1ccc(-c2cn(CC(=O)O[C@H]3[C@H](C)[C@H](O)[C@H](C)[C@@H](O)[C@@H](C)/C=C/C=C(/C)C(=O)NC4=C5NC6(CCN(CC(C)C)CC6)N=C5c5c(c(O)c(C)c6c5C(=O)[C@@](C)(O/C=C/[C@H](OC)[C@H]3C)O6)C4=O)nn2)cc1] |
| 3 | 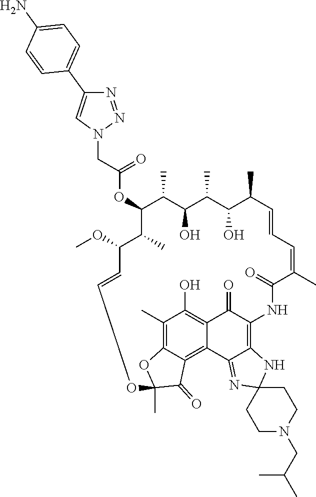 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(-c3ccc(N)cc3)nn2)[C@@H]1C)C4=O] |
| 4 | 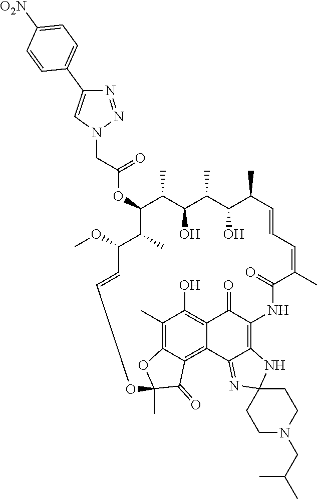 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(-c3ccc([N+](=O)[O-])cc3)nn2)[C@@H]1C)C4=O] |
| 5 | 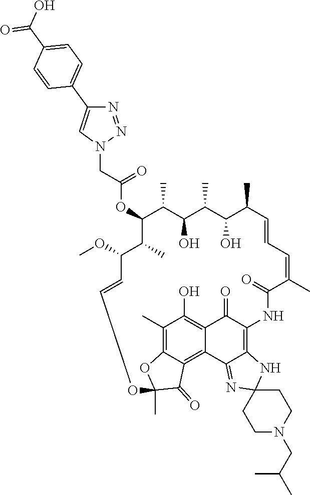 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(-c3ccc(C(=O)O)cc3)nn2)[C@@H]1C)C4=O] |
| 6 | 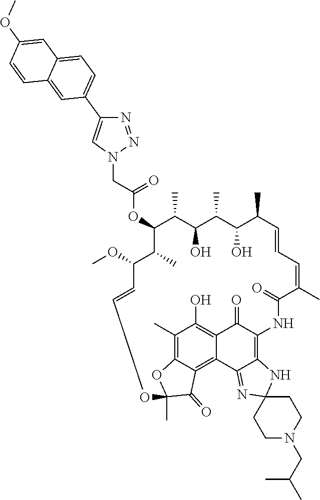 [SMILES: COc1ccc2cc(-c3cn(CC(=O)O[C@H]4[C@H](C)[C@H](O)[C@H](C)[C@@H](O)[C@@H](C)/C=C/C=C(/C)C(=O)NC5=C6NC7(CCN(CC(C)C)CC7)N=C6c6c(c(O)c(C)c7c6C(=O)[C@@](C)(O/C=C/[C@H](OC)[C@H]4C)O7)C5=O)nn3)ccc2c1] |
| 7 | 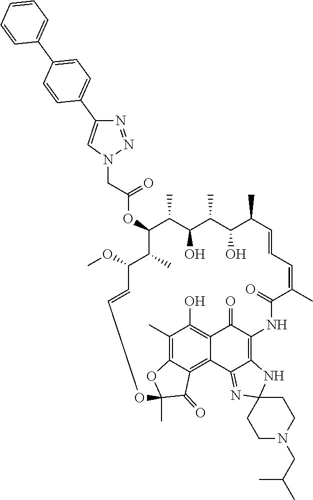 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(-c3ccc(-c5ccccc5)cc3)nn2)[C@@H]1C)C4=O] |
| 8 | 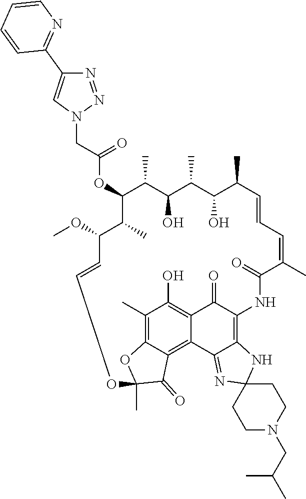 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(-c3ccccn3)nn2)[C@@H]1C)C4=O] |
| 9 | 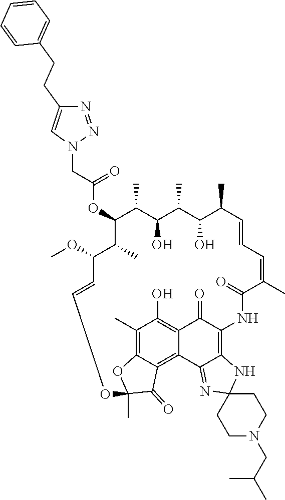 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CCc3ccccc3)nn2)[C@@H]1C)C4=O] |
| 10 | 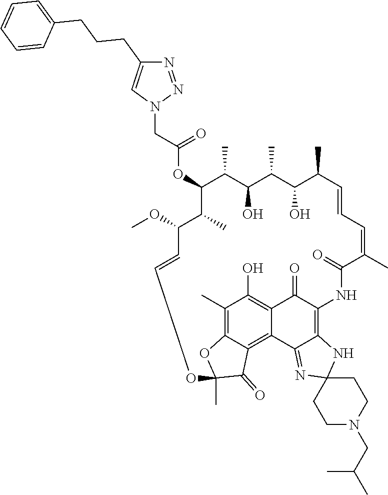 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CCCc3ccccc3)nn2)[C@@H]1C)C4=O] |
| 11 | 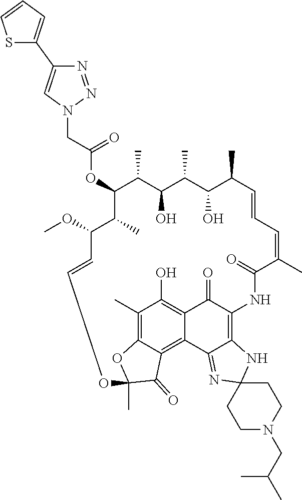 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(-c3cccs3)nn2)[C@@H]1C)C4=O] |
| 12 | 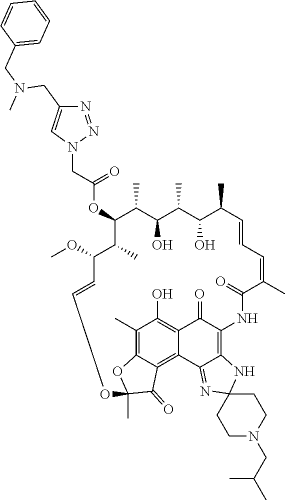 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CN(C)Cc3ccccc3)nn2)[C@@H]1C)C4=O] |
| 13 | 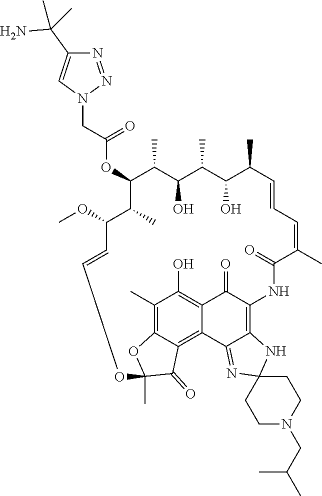 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(C(C)(C)N)nn2)[C@@H]1C)C4=O] |
| 14 | 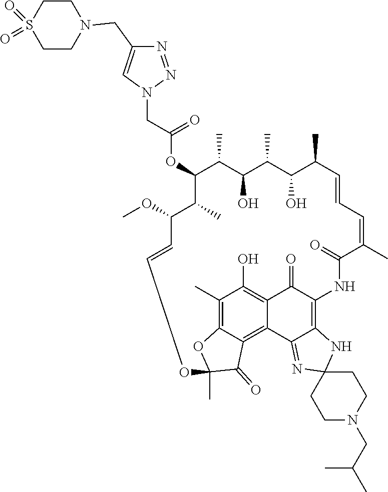 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CN3CCS(=O)(=O)CC3)nn2)[C@@H]1C)C4=O] |
| 15 | 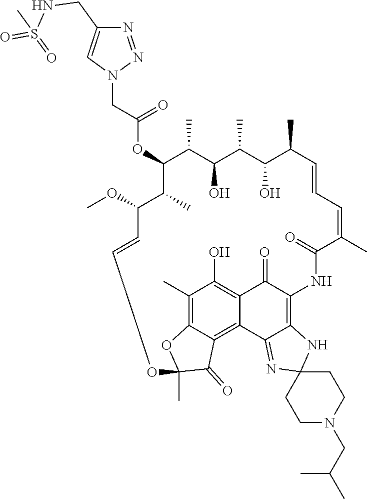 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CNS(C)(=O)=O)nn2)[C@@H]1C)C4=O] |
| 16 | 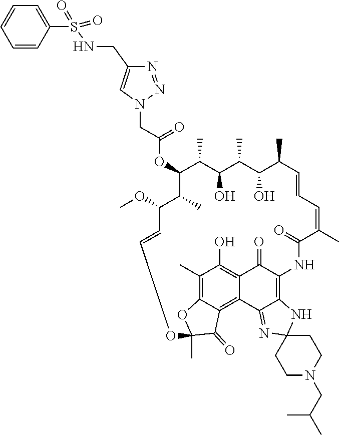 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CNS(=O)(=O)c3ccccc3)nn2)[C@@H]1C)C4=O] |
| 17 | 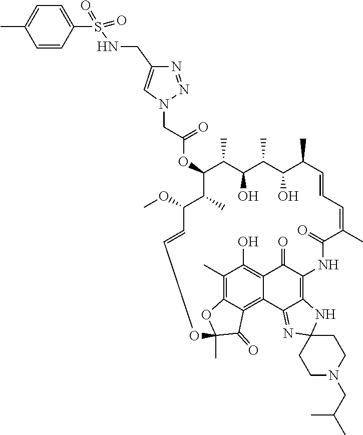 |
| 18 | 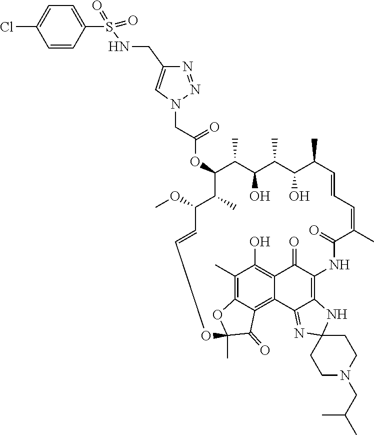 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CNS(=O)(=O)c3ccc(Cl)cc3)nn2)[C@@H]1C)C4=O] |
| 19 | 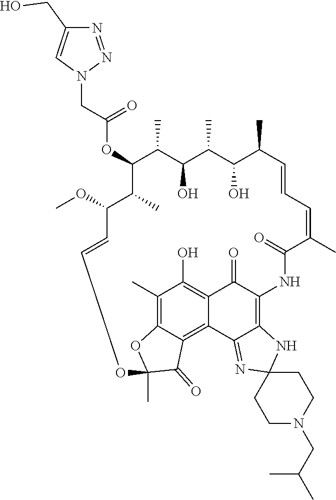 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CO)nn2)[C@@H]1C)C4=O] |
| 20 | 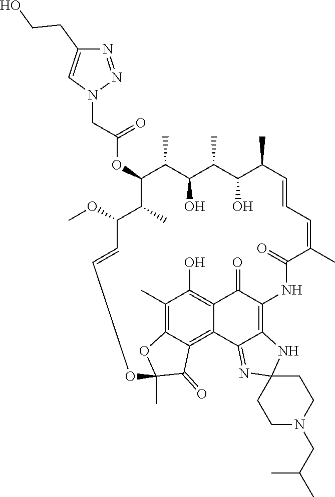 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CCO)nn2)[C@@H]1C)C4=O] |
| 21 | 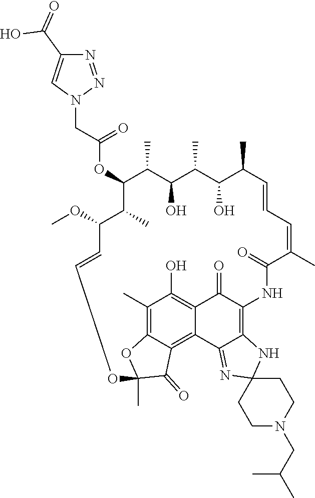 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(C(=O)O)nn2)[C@@H]1C)C4=O] |
| 22 | 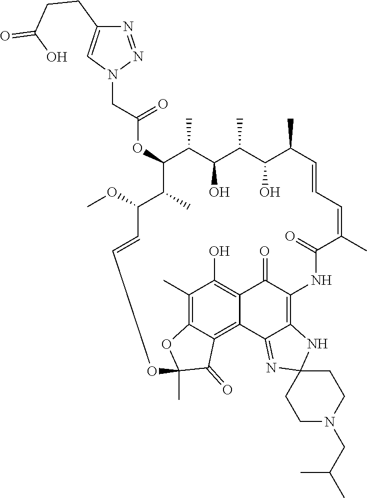 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CCC(=O)O)nn2)[C@@H]1C)C4=O] |
| 23 | 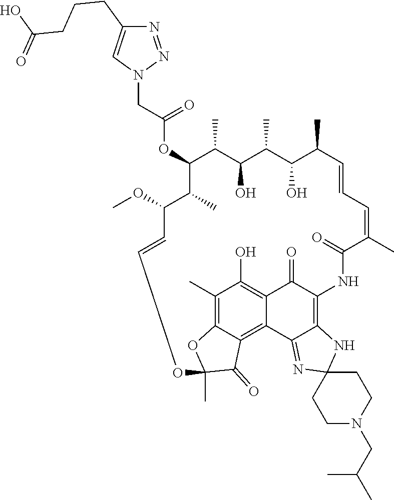 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CCCC(=O)O)nn2)[C@@H]1C)C4=O] |
| 24 | 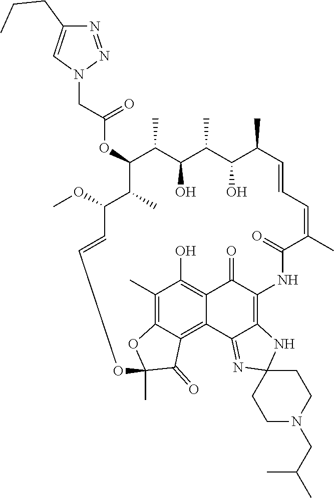 [SMILES: CCCc1cn(CC(=O)O[C@H]2[C@H](C)[C@H](O)[C@H](C)[C@@H](O)[C@@H](C)/C=C/C=C(/C)C(=O)NC3=C4NC5(CCN(CC(C)C)CC5)N=C4c4c(c(O)c(C)c5c4C(=O)[C@@](C)(O/C=C/[C@H](OC)[C@H]2C)O5)C3=O)nn1] |
| 25 | 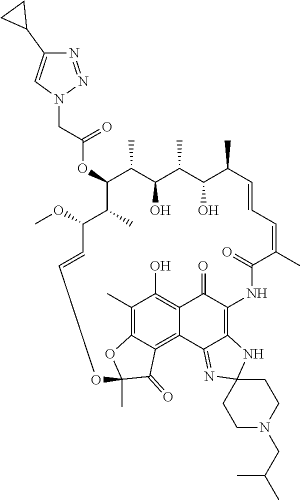 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(C3CC3)nn2)[C@@H]1C)C4=O] |
| 26 | 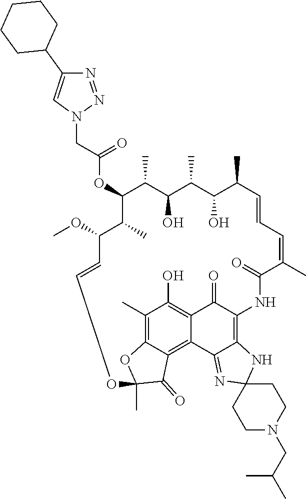 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(C3CCCCC3)nn2)[C@@H]1C)C4=O] |
| 27 | 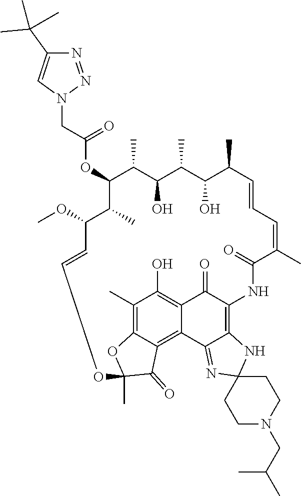 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(C(C)(C)C)nn2)[C@@H]1C)C4=O] |
| 28 | 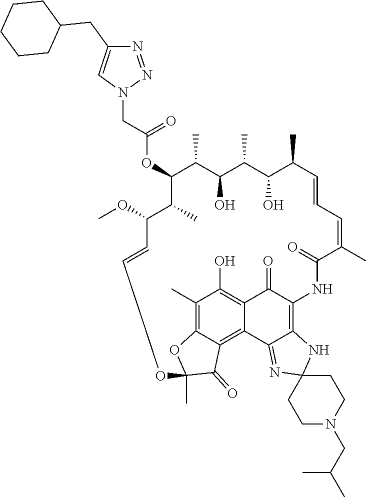 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CC3CCCCC3)nn2)[C@@H]1C)C4=O] |
| 29 | 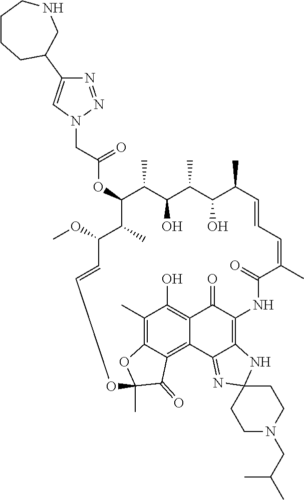 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(C3CCCCNC3)nn2)[C@@H]1C)C4=O] |
| 30 | 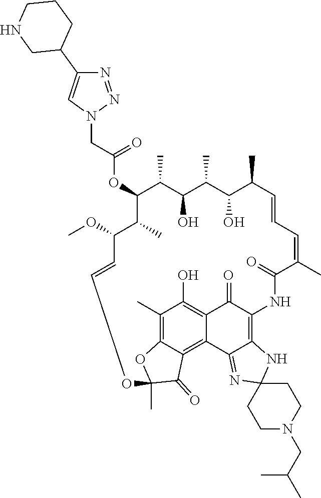 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(C3CCCNC3)nn2)[C@@H]1C)C4=O] |
| 31 | 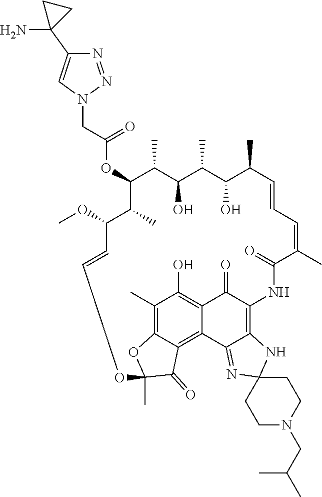 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(C3(N)CC3)nn2)[C@@H]1C)C4=O] |
| 32 | 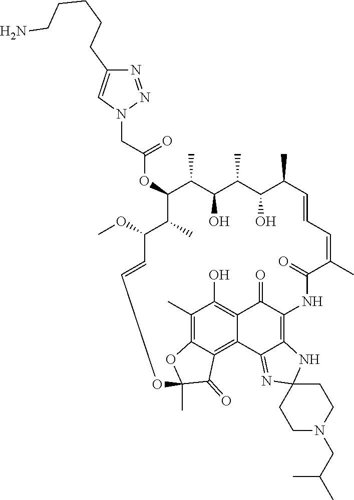 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CCCCCN)nn2)[C@@H]1C)C4=O] |
| 33 | 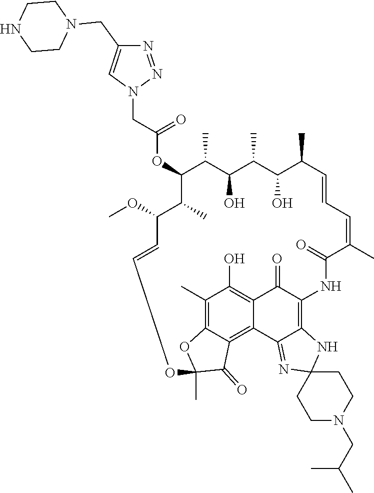 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CN3CCNCC3)nn2)[C@@H]1C)C4=O] |
| 34 | 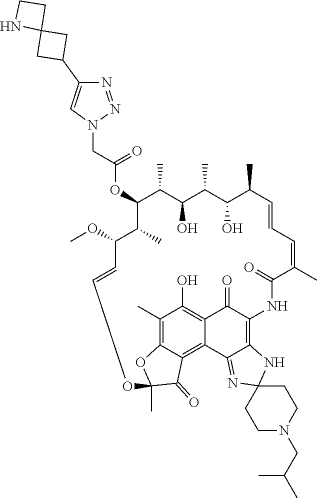 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(C3CC5(CCN5)C3)nn2)[C@@H]1C)C4=O] |

In one or more embodiments of any of the above aspects, the compound of Formula (I) is selected from a compound of Table 3, below:

TABLE 3
Compounds of the Invention

| Cpd | Structure |
|---|---|
| 1 | 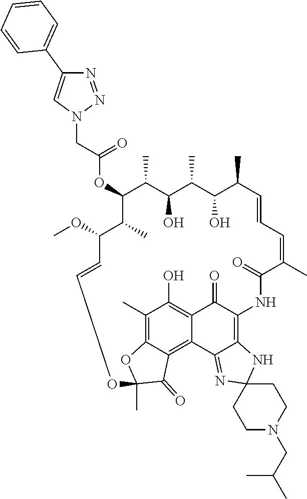 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(-c3ccccc3)nn2)[C@@H]1C)C4=O] |
| 2 | 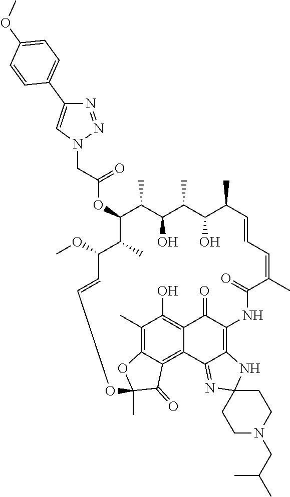 [SMILES: COc1ccc(-c2cn(CC(=O)O[C@H]3[C@H](C)[C@H](O)[C@H](C)[C@@H](O)[C@@H](C)/C=C/C=C(/C)C(=O)NC4=C5NC6(CCN(CC(C)C)CC6)N=C5c5c(c(O)c(C)c6c5C(=O)[C@@](C)(O/C=C/[C@H](OC)[C@H]3C)O6)C4=O)nn2)cc1] |
| 3 | 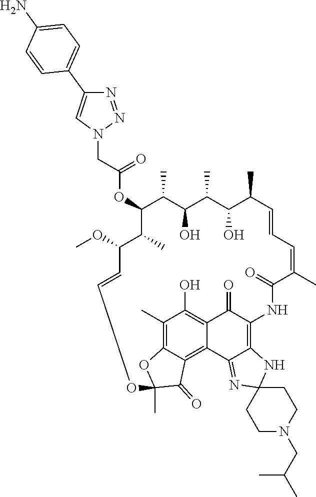 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(-c3ccc(N)cc3)nn2)[C@@H]1C)C4=O] |
| 4 | 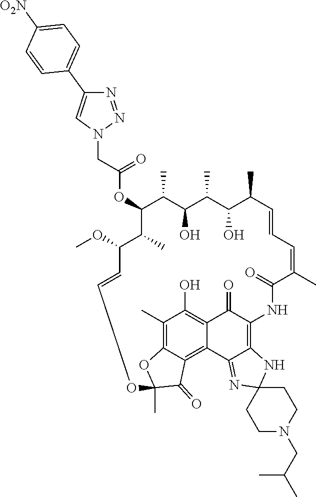 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(-c3ccc([N+](=O)[O-])cc3)nn2)[C@@H]1C)C4=O] |
| 5 | 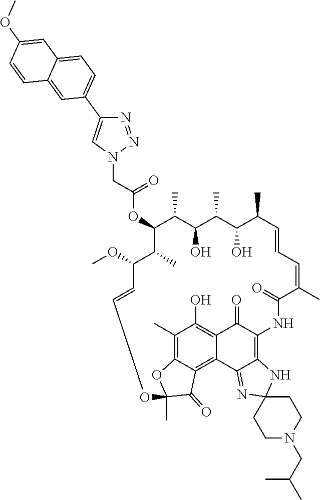 [SMILES: COc1ccc2cc(-c3cn(CC(=O)O[C@H]4[C@H](C)[C@H](O)[C@H](C)[C@@H](O)[C@@H](C)/C=C/C=C(/C)C(=O)NC5=C6NC7(CCN(CC(C)C)CC7)N=C6c6c(c(O)c(C)c7c6C(=O)[C@@](C)(O/C=C/[C@H](OC)[C@H]4C)O7)C5=O)nn3)ccc2c1] |
| 8 | 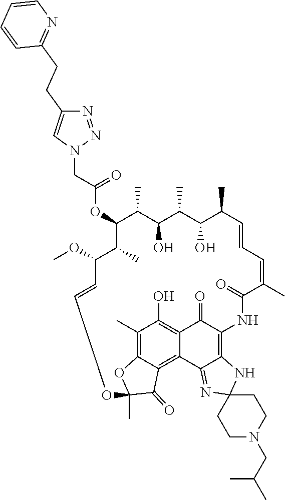 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CCc3ccccn3)nn2)[C@@H]1C)C4=O] |
| 9 | 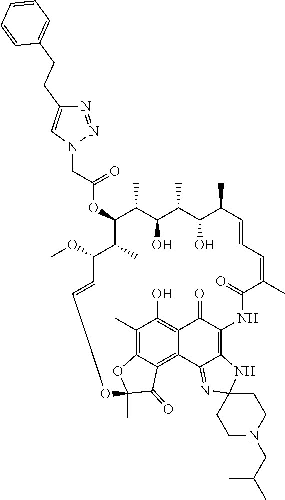 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CCc3ccccc3)nn2)[C@@H]1C)C4=O] |
| 10 | 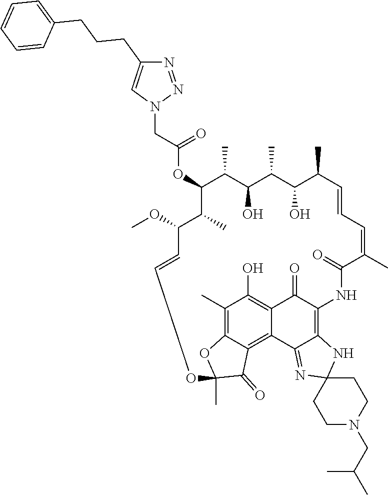 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CCCc3ccccc3)nn2)[C@@H]1C)C4=O] |
| 11 | 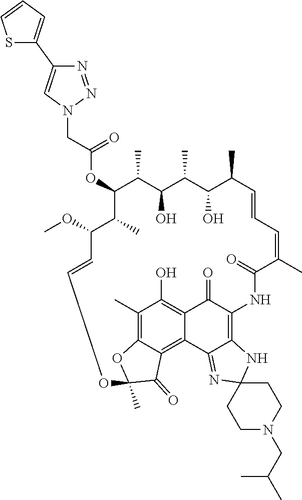 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(-c3cccs3)nn2)[C@@H]1C)C4=O] |
| 12 | 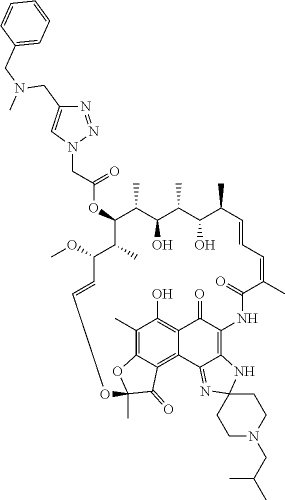 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CN(C)Cc3ccccc3)nn2)[C@@H]1C)C4=O] |
| 13 | 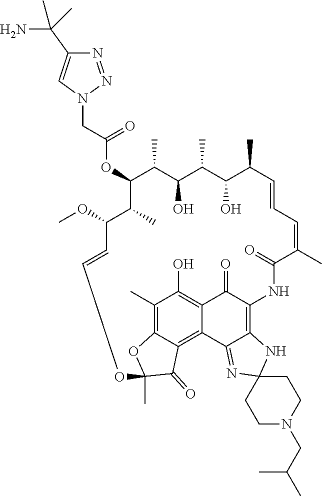 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(C(C)(C)N)nn2)[C@@H]1C)C4=O] |
| 14 | 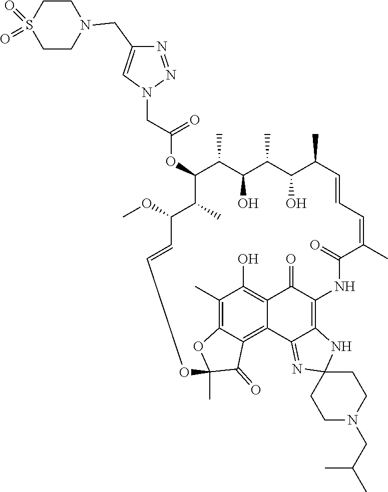 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CN3CCS(=O)(=O)CC3)nn2)[C@@H]1C)C4=O] |
| 15 | 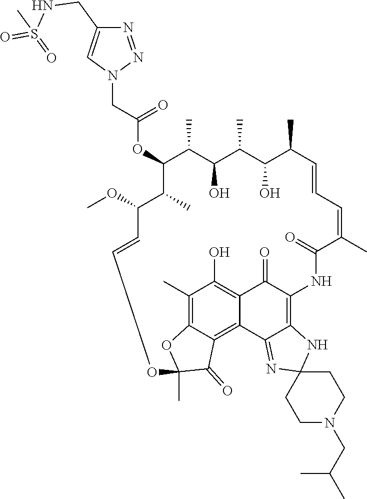 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CNS(C)(=O)=O)nn2)[C@@H]1C)C4=O] |
| 16 | 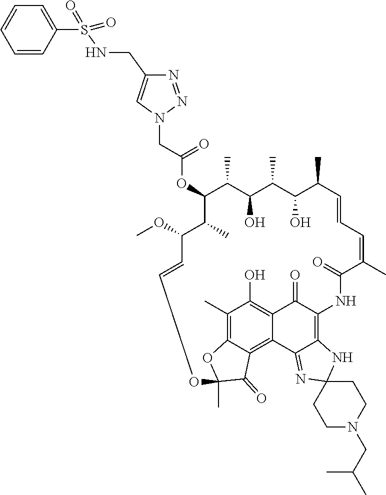 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CNS(=O)(=O)c3ccccc3)nn2)[C@@H]1C)C4=O] |
| 17 | 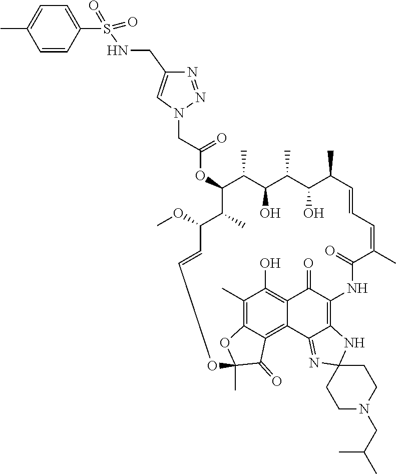 |
| 18 | 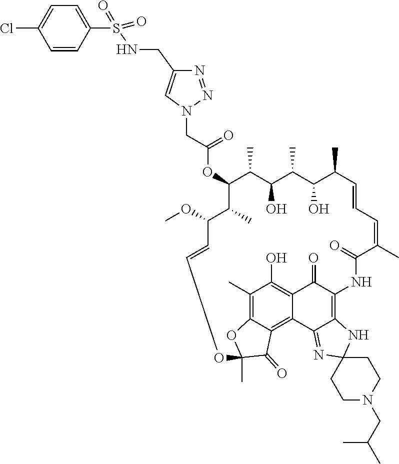 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CNS(=O)(=O)c3ccc(Cl)cc3)nn2)[C@@H]1C)C4=O] |
| 24 | 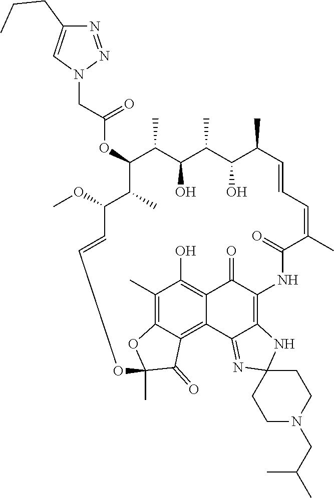 [SMILES: CCCc1cn(CC(=O)O[C@H]2[C@H](C)[C@H](O)[C@H](C)[C@@H](O)[C@@H](C)/C=C/C=C(/C)C(=O)NC3=C4NC5(CCN(CC(C)C)CC5)N=C4c4c(c(O)c(C)c5c4C(=O)[C@@](C)(O/C=C/[C@H](OC)[C@H]2C)O5)C3=O)nn1] |
| 26 | 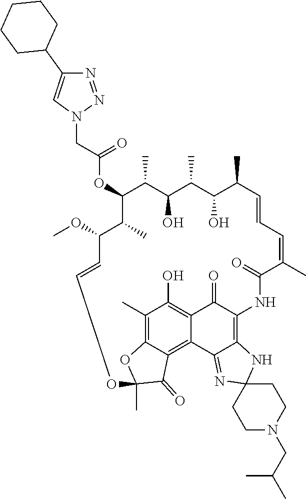 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(C3CCCCC3)nn2)[C@@H]1C)C4=O] |
| 28 | 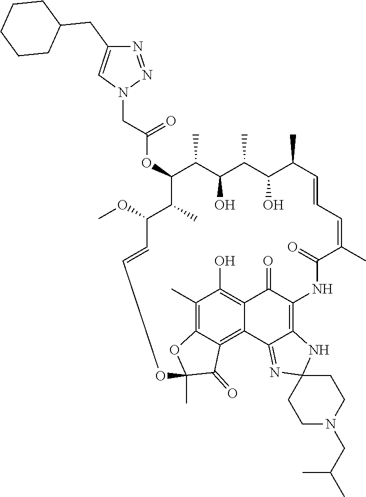 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CC3CCCCC3)nn2)[C@@H]1C)C4=O] |

In one or more embodiments of any of the above aspects, the compound of Formula (I) is selected from a compound of Table 4, below:

TABLE 4
Compounds of the Invention

| Cpd | Structure |
|---|---|
| 1 | 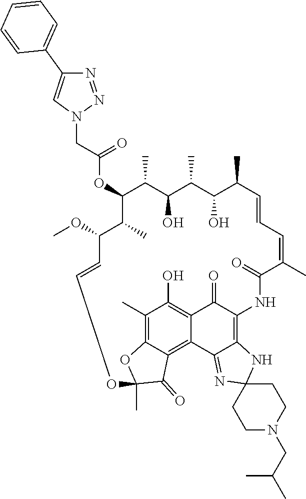 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(-c3ccccc3)nn2)[C@@H]1C)C4=O] |
| 2 | 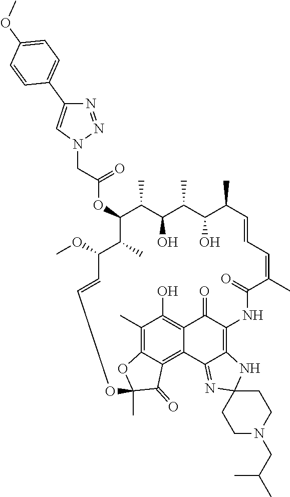 [SMILES: COc1ccc(-c2cn(CC(=O)O[C@H]3[C@H](C)[C@H](O)[C@H](C)[C@@H](O)[C@@H](C)/C=C/C=C(/C)C(=O)NC4=C5NC6(CCN(CC(C)C)CC6)N=C5c5c(c(O)c(C)c6c5C(=O)[C@@](C)(O/C=C/[C@H](OC)[C@H]3C)O6)C4=O)nn2)cc1] |
| 4 | 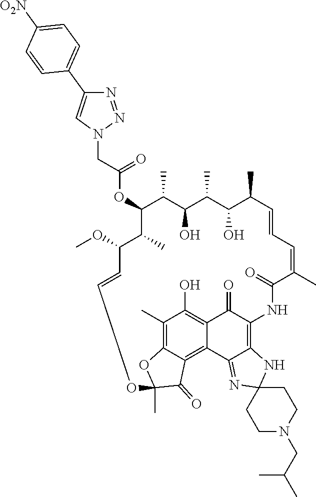 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(-c3ccc([N+](=O)[O-])cc3)nn2)[C@@H]1C)C4=O] |
| 8 | 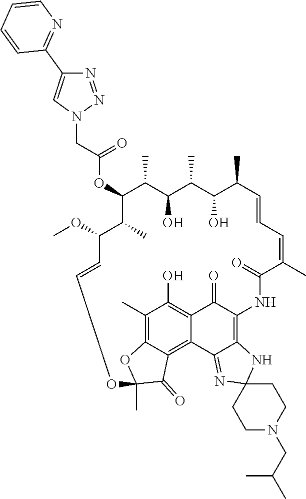 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(-c3ccccn3)nn2)[C@@H]1C)C4=O] |
| 9 | 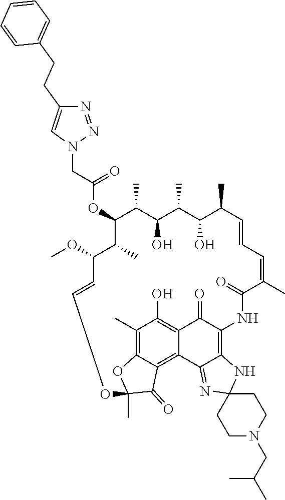 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CCc3ccccc3)nn2)[C@@H]1C)C4=O] |
| 12 | 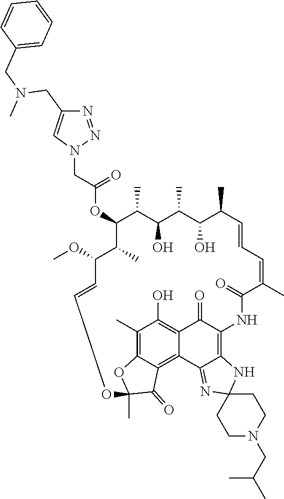 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CN(C)Cc3ccccc3)nn2)[C@@H]1C)C4=O] |
| 16 | 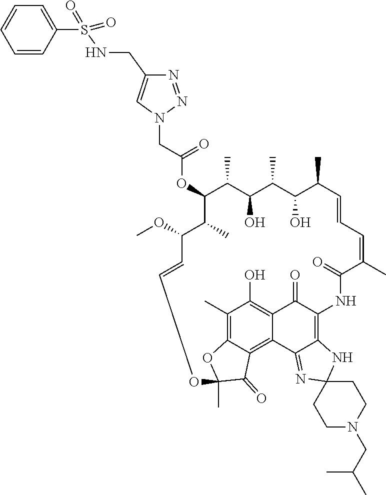 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CNS(=O)(=O)c3ccccc3)nn2)[C@@H]1C)C4=O] |
| 17 | 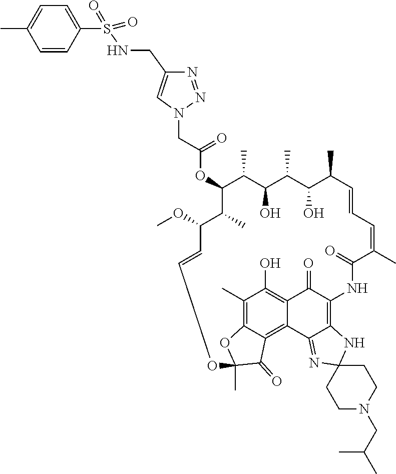 |
| 18 | 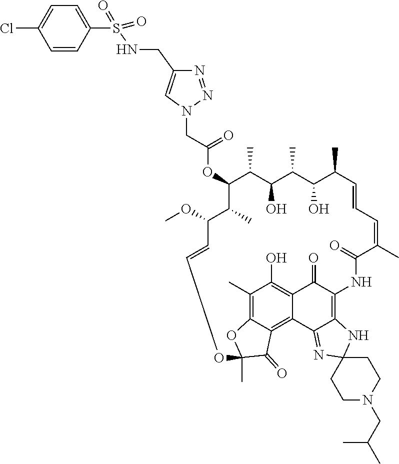 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CNS(=O)(=O)c3ccc(Cl)cc3)nn2)[C@@H]1C)C4=O] |
| 26 | 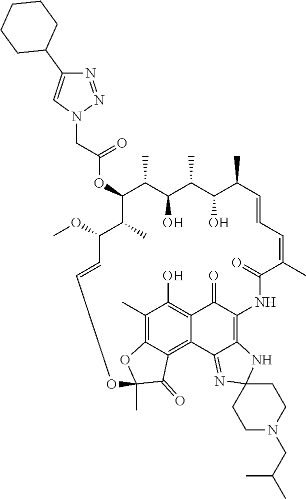 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(C3CCCCC3)nn2)[C@@H]1C)C4=O] |
| 28 | 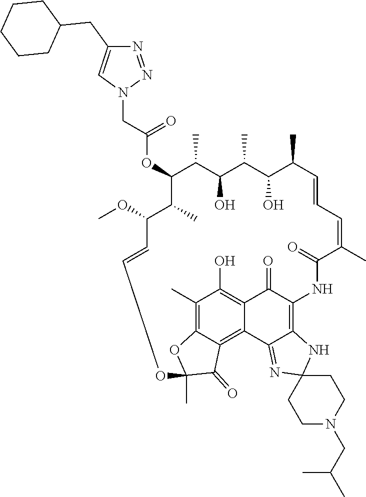 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CC3CCCCC3)nn2)[C@@H]1C)C4=O] |

In some embodiments, the compound of Formula (I) is selected from the group consisting of Compound 1, Compound 2, Compound 3, Compound 4, Compound 6, Compound 7, Compound 8, Compound 9, Compound 10, Compound 11, Compound 12, Compound 13, Compound 14, Compound 15, Compound 16, Compound 17, Compound 18, Compound 24, Compound 26, and Compound 28.

In some embodiments, the compound of Formula (I) is selected from Compound 1, Compound 2, Compound 3, Compound 4, Compound 6, Compound 8, Compound 9, Compound 10, Compound 11, Compound 12, Compound 13, Compound 14, Compound 15, Compound 16, Compound 17, Compound 18, Compound 24, Compound 26, and Compound 28.

In some embodiments, the compound of Formula (I) is selected from Compound 1, Compound 2, Compound 4, Compound 8, Compound 9, Compound 12, Compound 16, Compound 17, Compound 18, Compound 26, and Compound 28.

In some embodiments, the compound of Formula (I) is Compound 1. In some embodiments, the compound of Formula (I) is Compound 2. In some embodiments, the compound of Formula (I) is Compound 3. In some embodiments, the compound of Formula (I) is Compound 4. In some embodiments, the compound of Formula (I) is Compound 5. In some embodiments, the compound of Formula (I) is Compound 6. In some embodiments, the compound of Formula (I) is Compound 7. In some embodiments, the compound of Formula (I) is Compound 8. In some embodiments, the compound of Formula (I) is Compound 9. In some embodiments, the compound of Formula (I) is Compound 10. In some embodiments, the compound of Formula (I) is Compound 11. In some embodiments, the compound of Formula (I) is Compound 12. In some embodiments, the compound of Formula (I) is Compound 13. In some embodiments, the compound of Formula (I) is Compound 14. In some embodiments, the compound of Formula (I) is Compound 15. In some embodiments, the compound of Formula (I) is Compound 16. In some embodiments, the compound of Formula (I) is Compound 17. In some embodiments, the compound of Formula (I) is Compound 18. In some embodiments, the compound of Formula (I) is Compound 19. In some embodiments, the compound of Formula (I) is Compound 20. In some embodiments, the compound of Formula (I) is Compound 21. In some embodiments, the compound of Formula (I) is Compound 22. In some embodiments, the compound of Formula (I) is Compound 23. In some embodiments, the compound of Formula (I) is Compound 24. In some embodiments, the compound of Formula (I) is Compound 25. In some embodiments, the compound of Formula (I) is Compound 26. In some embodiments, the compound of Formula (I) is Compound 27. In some embodiments, the compound of Formula (I) is Compound 28. In some embodiments, the compound of Formula (I) is Compound 29. In some embodiments, the compound of Formula (I) is Compound 30. In some embodiments, the compound of Formula (I) is Compound 31. In some embodiments, the compound of Formula (I) is Compound 32. In some embodiments, the compound of Formula (I) is Compound 33. In some embodiments, the compound of Formula (I) is Compound 34.

In one aspect, the present invention provides a pharmaceutical composition comprising at least one compound according to the present invention, or a pharmaceutically acceptable salt, tautomer, solvate or hydrate thereof, and a pharmaceutically acceptable excipient.

### Methods of Synthesizing the Disclosed Compounds

The compounds of the present invention may be made by a variety of methods, including standard chemistry. The methods include but are not limited to the methods described in the suitable synthetic routes depicted in the schemes given below.

The compounds of the present invention may be prepared by methods known in the art of organic synthesis as set forth in part by the following synthetic schemes and examples. In the schemes described below, it is well understood that protecting groups for sensitive or reactive groups are employed where necessary in accordance with general principles or chemistry. Protecting groups are manipulated according to standard methods of organic synthesis (T. W. Greene and P. G. M. Wuts, “Protective Groups in Organic Synthesis”, Third edition, Wiley, New York 1999). These groups are removed at a convenient stage of the compound synthesis using methods that are readily apparent to those skilled in the art. The selection processes, as well as the reaction conditions and order of their execution, shall be consistent with the preparation of compounds of the present invention.

Those skilled in the art will recognize if a stereocenter exists in the compounds of Formula (I). Accordingly, the present invention includes both possible stereoisomers (unless specified in the synthesis) and includes not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley—Interscience, 1994).

The compounds described herein may be made from commercially available starting materials or synthesized using known organic, inorganic, and/or enzymatic processes.

### Preparation of Compounds

Compounds of the present invention can be synthesized by following the steps outlined in Schemes 1, 2, 3 and 4. Starting materials are either commercially available or made by known procedures in the reported literature or as illustrated.

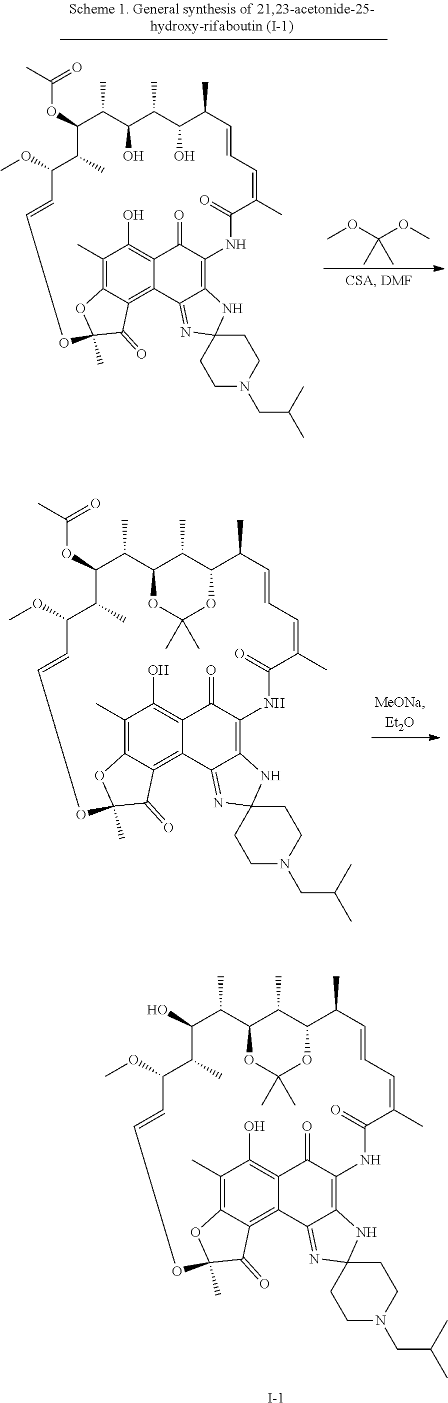

A general method for the preparation of 21,23-acetonide-25-hydroxy-rifabutin I-1 is shown above in Scheme 1. Protection of the C21 and C23 hydroxy groups using a suitable protecting group such as dimethoxy propane and camphorsulphonic acid in a solvent, e.g., DMF afforded an acetal-protected rifabutin. De-acetylation of the protected rifabutin using a basic solution such as sodium methoxide in ether afforded the 25-OH rifabutin with the C21 and C23 hydroxy groups protected (I-1).

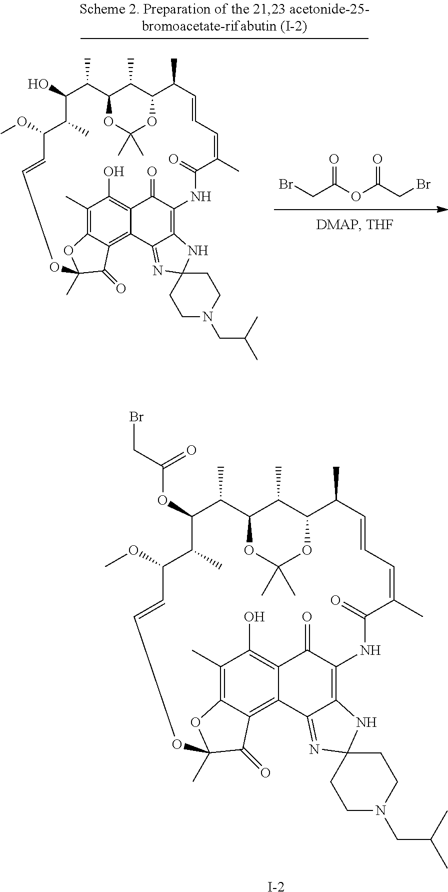

21,23-Acetonide-25-hydroxy-rifabutin (I-1) can be esterified using an appropriate carboxylic acid anhydride such as bromoacetic anhydride in the presence of a suitable base such as an amine base, e.g., 4-(dimethylamino)pyridine, as shown in Scheme 2.

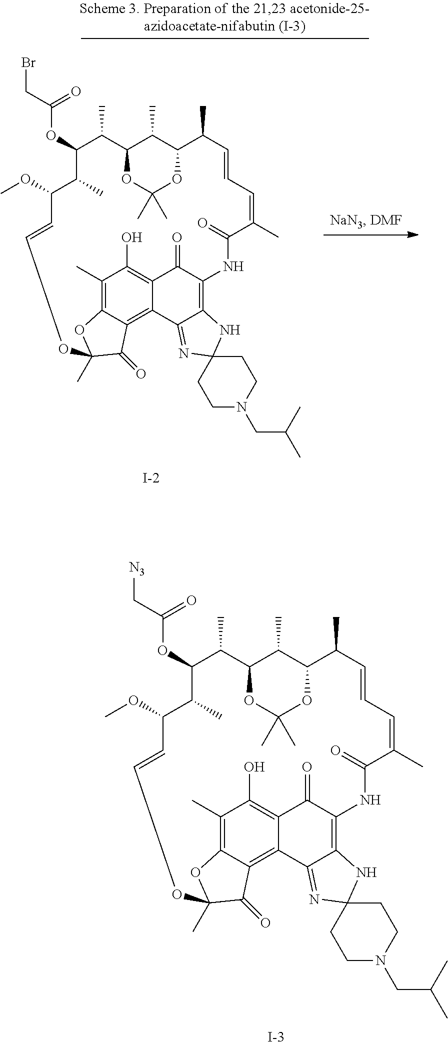

The intermediate I-2 can be converted into the corresponding azide by reaction with a suitable nucleophilic azide, e.g., sodium azide in a solvent such as DMF, as shown in Scheme 3.

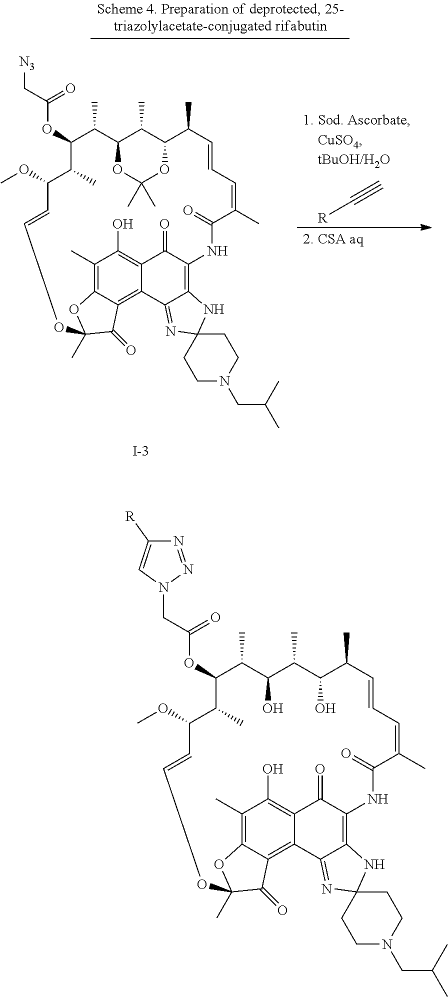

The C25-esterified, protected rifabutin can be condensed with an appropriate alkyne in the presence of an appropriate catalyst such as copper sulfate and sodium ascorbate in a suitable solvent such as a mixture of tBuOH and water. Deprotection of the C21-C23 acetonide in the conjugated rifabutin can be carried out by treating with an acid such as camphorsulphonic acid in water.

### Antibacterial Efficacy of the Disclosed Compounds

The inventive compounds are analogs of rifabutin modified at C₂₅ to comprise a 2-triazolo acetate ester, wherein said triazole is substituted at the 4-position. The inventive compounds exhibited broad spectrum antibacterial activity characteristic of the rifamycin class. Additionally, the inventive compounds unexpectedly showed enhanced antibacterial activity against non-tuberculous Mycobacteria including M. abscessus compared to currently available antibiotics (e.g., rifabutin).

As shown below in Example 5, Table 6, the compounds of the invention are effective at inhibiting bacterial growth in strains of S. aureus, M. abscessus, A. baumannii, M. kansasii, M. xenopi and M. avium.

Rifampicin exhibited MIC value above 32 mg/L on M. abscessus and was not considered active against M. abscessus strains. Rifabutin, which is not modified on its C25 position, exhibited modest activity against the tested M. abscessus strains with an MIC value of 8 mg/L. In contrast, Compounds of the invention showed MIC values from 0.125 to 4 mg/L, corresponding to 2 to 64-fold increased activity over rifabutin. Accordingly, the present invention teaches compounds that display increased activity against M. abscessus beyond that of antibiotics known in the literature.

### Methods of Using the Disclosed Compounds

An aspect of the present invention relates to a compound of Formula (I) or a pharmaceutically acceptable salt thereof for use as a medicament. An aspect of the present invention relates to a pharmaceutical composition comprising a compound of Formula (I) or a pharmaceutically acceptable salt thereof for use as a medicament.

In one aspect, the present invention provides a compound of Formula (I) or a pharmaceutically acceptable salt, tautomer, solvate or hydrate thereof, or a pharmaceutical composition comprising a compound of Formula (I), for use in a method of preventing or treating a disease in a subject, preferably an infection, further preferably a bacterial infection.

In one aspect, the present invention provides a method of treating a disease, preferably an infection, further preferably a bacterial infection in a subject in need thereof, the method comprising administering to the subject an effective amount of a compound of Formula (I) or a pharmaceutically acceptable salt, tautomer, solvate, or hydrate thereof, or a pharmaceutical composition comprising a compound of Formula (I).

In one aspect, the present invention provides the use of a compound of Formula (I) or a pharmaceutically acceptable salt, tautomer, solvate, or hydrate thereof in the manufacture of a medicament for treating a disease, preferably an infection, further preferably a bacterial infection in a subject in need thereof.

In some embodiments of any of the above-aspects, the infection, e.g., the bacterial infection, is caused by one or more bacterium belonging to the genera Mycobacterium spp., Acinetobacter spp., Clostridium spp., Enterococcus spp., Hemophilus spp., Legionella spp., Neisseria spp., Staphylococcus spp., Streptococcus spp., Listeria monocytogenes, Moraxella catarrhalis, Bacillus spp., Bacteroides spp., Gardnerella vaginalis, Lactobacillus spp., Mobiluncus spp., Helicobacter pylori, Campylobacter jejuni, Chlamydia trachomatis and/or Toxoplasma gondii.

In some preferred embodiments of any of the above-aspects, the bacterial infection is caused by one or more bacterium belonging to the genus Acinetobacter, Staphylococcus, and/or Mycobacteria. In some preferred embodiments, the bacterial infection is caused by one or more bacterium belonging to the species A. baumannii, and/or S. aureus, and/or a genus of non-tuberculous Mycobacteria, preferably M. abscessus. In some embodiments, the infection is caused by one or more bacterium belonging to the species M. abscessus, A. baumannii, and/or S. aureus, preferably M. abscessus.

In some preferred embodiments, the infection is caused by one or more bacterium belonging to a genus of non-tuberculosis Mycobacterium, preferably M. abscessus, M avium, M. kansasii, M. smegmantis, M. xenopi and/or M. malmoense, more preferably M. abscessus, M. avium, M. kansasii, and/or M. xenopi, yet more preferably M. abscessus. In some embodiments, the M. abscessus infection is resistant to current antibiotics.

In some embodiments, the infection is caused by one or more bacterium belonging to the genus Acinetobacter and/or Staphylococcus, preferably A. baumannii and/or S. aureus. In some embodiments, the infection is caused by one or more bacterium belonging to the genus Acinetobacter, preferably A. baumannii. In some embodiments, the infection is caused by one or more bacterium belonging to the genus Staphylococcus, preferably S. aureus.

In one aspect, the present invention provides a compound of Formula (I) or a pharmaceutically acceptable salt, tautomer, solvate, or hydrate thereof, or a pharmaceutical composition comprising a compound of Formula (I), for use in a method of treating a non-tuberculous Mycobacteria pulmonary infection. In some embodiments, the bacteria causing the non-tuberculous Mycobacteria pulmonary infection is M. abscessus, M. avium, M. kansasii, M. smegmantis, M. xenopi and/or M. malmoense, preferably M. abscessus, M. avium, M. kansasii, and/or M. xenopi, more preferably M. abscessus.

In one aspect, the present invention provides a method of treating a non-tuberculous Mycobacteria pulmonary infection in a subject in need thereof, the method comprising administering to the subject an effective amount of a compound of Formula (I) or a pharmaceutically acceptable salt, tautomer, solvate, or hydrate thereof, or a pharmaceutical composition comprising a compound of Formula (I). In some embodiments, the bacteria causing the non-tuberculous Mycobacteria pulmonary infection is M. abscessus, M avium, M kansasii, M. smegmantis, M. xenopi and/or M. malmoense, preferably M. abscessus, M avium, M. kansasii, and or M. xenopi, more preferably M. abscessus.

In one aspect, the present invention provides the use of a compound of Formula (I) or a pharmaceutically acceptable salt, tautomer, solvate, or hydrate thereof, in the manufacture of a medicament for treating a non-tuberculous Mycobacteria pulmonary infection in a subject in need thereof. In some embodiments, the bacteria causing the non-tuberculous Mycobacteria pulmonary infection is M. abscessus, M. avium, M. kansasii, M. smegmantis, M. xenopi and/or M. malmoense, preferably M. abscessus, M. avium, M. kansasii, and or M. xenopi, more preferably M. abscessus.

In one aspect, the present invention provides a pharmaceutical composition comprising at least one compound according to Formula (I), or a pharmaceutically acceptable salt, tautomer, solvate or hydrate thereof, and a pharmaceutically acceptable excipient.

Illustrative pharmaceutical compositions are tablets and gelatin capsules comprising a compound of the invention and a pharmaceutically acceptable carrier, such as a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, algiic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, PEG200.

The inventive compounds and pharmaceutical compositions may be administered by any suitable route, e.g. orally, for example as a syrup, tablet, capsule, lozenge, controlled-release preparation, fast-dissolving preparation, or lozenge.

Liquid, particularly injectable, compositions can, for example, be prepared by dissolution, dispersion, etc. For example, the disclosed compound is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the disclosed compounds.

The disclosed compounds can be also formulated as a suppository that can be prepared from fatty emulsions or suspensions, using polyalkylene glycols such as propylene glycol, as the carrier.

The disclosed compounds can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564.

Parental injectable administration is generally used for subcutaneous, intramuscular or intravenous injections and infusions. Injectables can be prepared in conventional forms, either as liquid solutions or suspensions or solid forms suitable for dissolving in liquid prior to injection.

Another aspect of the invention relates to a pharmaceutical composition comprising a compound of the present invention and a pharmaceutically acceptable carrier. The pharmaceutically acceptable carrier can further include an excipient, diluent, or surfactant.

Compositions can be prepared according to conventional mixing, granulating or coating methods, respectively, and the present pharmaceutical compositions can contain from about 0.1% to about 99%, from about 5% to about 90%, or from about 1% to about 20% of the disclosed compound by weight or volume.

The dosage regimen utilizing the disclosed compound is selected in accordance with a variety of factors including type, species, age, weight, sex and medical condition of the patient; the severity of the condition to be treated; the route of administration; the renal or hepatic function of the patient; and the particular disclosed compound employed. A physician or veterinarian of ordinary skill in the art can readily determine and prescribe the effective amount of the drug required to prevent, counter, or arrest the progress of the condition.

Effective dosage amounts of the disclosed compounds, when used for the indicated effects, range from about 0.5 mg to about 5000 mg of the disclosed compound as needed to treat the condition. Compositions for in vivo or in vitro use can contain about 0.5, 5, 20, 50, 75, 100, 150, 250, 500, 750, 1000, 1250, 2500, 3500, or 5000 mg of the disclosed compound, or, in a range of from one amount to another amount in the list of doses. In one embodiment, the compositions are in the form of a tablet that can be scored.

### EQUIVALENTS

While the present technology has been described in conjunction with the specific embodiments set forth above, many alternatives, modifications and other variations thereof will be apparent to those of ordinary skill in the art. All such alternatives, modifications and variations are intended to fall within the spirit and scope of the present invention.

### EXAMPLES

The invention will now be illustrated by way of the following non-limiting examples. While particular embodiments of the invention are described below, a skilled person will appreciate that various changes and modifications can be made. References to preparations carried out in a similar manner to, or by the general method of, other preparations, may encompass variations in routine parameters such as time, temperature, workup conditions, minor changes in reagents amounts, and the like.

### Abbreviations

The following list provides definitions of certain abbreviations and symbols as used herein. It will be appreciated that the list is not exhaustive, but the meaning of those abbreviations and symbols not herein below defined will be readily apparent to those skilled in the art. In describing the invention, chemical elements are identified in accordance with the Periodic Table of the Elements.
ACN acetonitrile
AcOH acetic acid
aq. aqueous
Ar argon
CSA camphorsulphonic acid
DCM dichloromethane
DMF dimethylformamide
eq. equivalent
EtOAc ethyl acetate
EtOH ethanol
h hour(s)
HPLC high performance liquid chromatography
LC liquid chromatography
M molar
MeOH methanol
MS mass spectroscopy
min minutes
NMR nuclear magnetic resonance spectroscopy
rt room temperature
Rt retention time
sat. saturated

Unless specified otherwise, the purity and identity of Intermediate or example compounds were assessed by state-of-the-art HLPC-MS. Methods are described below.

### Characterization of the Compounds

Method A was used for the intermediates. Method B was used to measure the purity of the final compounds. Purity (%) was determined by reversed phase HPLC or UPLC, using UV detection (254 nm). Structure was confirmed by MS, using electro spray ionization positive (ESI+) method and reported as [M+H]⁺, referring to the protonated molecular ion.

### Method A

UPLC system: UPLC I BIN SOL MGR with ACQUITY UPLC I-Class eK PDA Detector; Column: Acquity BEH C18 column (1.7 μm particle size, dimensions 50 mm×2.1 mm); Mobile phases: phase A (H₂O/ammonium formate, pH 3.75 (A) or 9.2 (B)) and phase B (CH₃CN+5% H₂O/ammonium formate, pH 3.75 (A) or 9.2 (B)) were used according to the following methods:

Method A

| Time | | | Flow |
|---|---|---|---|
| (min) | Phase A | Phase B | (mL/min) |
| 0.00 | 98.0 | 2.0 | 0.6 |
| 0.20 | 98.0 | 2.0 | 0.6 |
| 2.50 | 0.0 | 100.0 | 0.6 |
| 3.50 | 0.0 | 100.0 | 0.6 |
| 3.60 | 98.0 | 2.0 | 0.6 |
| 5.00 | 98.0 | 2.0 | 0.6 |

Mass spectrometer: ACQUITY QDa (Performance) Xevo TQD. Ionization: electrospray (polarity: negative and positive).

### Method B

HPLC system: Waters 2695 LC with photodiode array detector Waters 996; Column: XBridge C18 column (3.5 μm particle size, dimensions 50 mm×4.6 mm); Mobile phases: phase A (H₂O/ammonium formate, pH 9.2) and phase B (CH₃CN+5% H₂O/ammonium formate, pH 9.2) were used according to the following methods:

Method B

| Time | | | Flow |
|---|---|---|---|
| (min) | Phase A | Phase B | (mL/min) |
| 0.00 | 100.0 | 0.0 | 2.00 |
| 1.00 | 100.0 | 0.0 | 2.00 |
| 25.00 | 0.0 | 100.0 | 2.00 |
| 27.00 | 0.0 | 100.0 | 2.00 |
| 27.10 | 100.0 | 0.0 | 2.00 |
| 30.00 | 100.0 | 0.0 | 2.00 |

Mass spectrometer: Waters Alliance Micromass ZQ 2000. Ionization: electrospray (polarity: negative and positive).

### NMR Analysis

NMR spectra were recorded on a Bruker DRX-300 spectrometer or Bruker 500 MHz spectrometer with a TXI probe. Chemical shifts are in parts per million (ppm). The assignments were made using one-dimensional (1D)¹H and ¹³C spectra and two-dimensional (2D) HSQC, HMBC spectra.

### Example 1

### Synthesis of 21,23-acetonide-25-hydroxy-rifabutin (I-1)

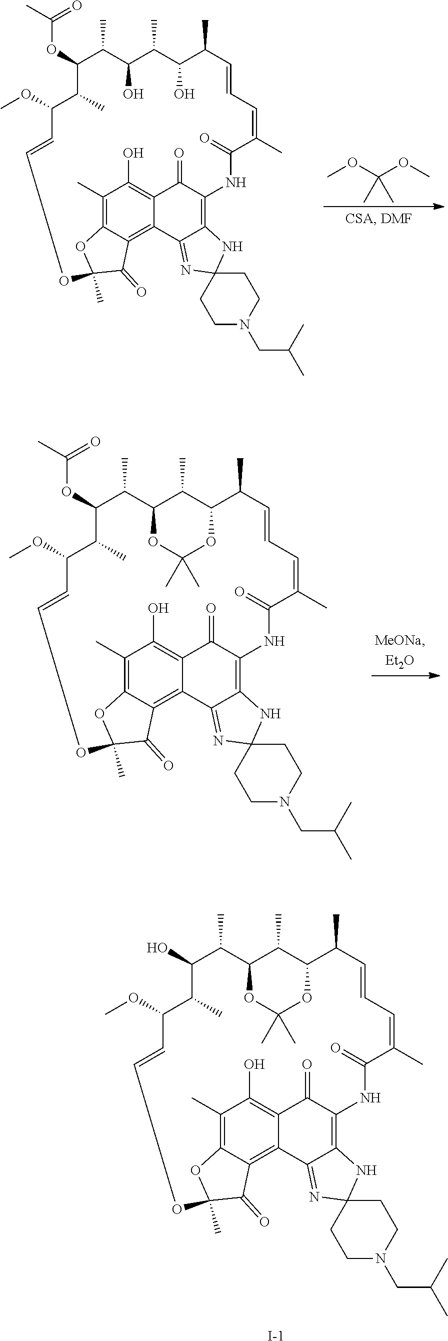

Commercially available rifabutin (40.0 g, 47.2 mmol) was dissolved in dry DMF (80 mL) at rt under nitrogen atmosphere. 2,2-Dimethoxy-propane (58.1 mL, 472 mmol) and camphorsulphonic acid (12.6 g, 54.3 mmol) were sequentially added to the solution. The reaction mixture was stirred at rt for 26 h under nitrogen atmosphere. The mixture was then cooled at 0° C. and poured into a mixture of sat. aq. solution of NaHCO₃ (700 mL) and water (500 mL). The reaction flask was washed with acetone (100 mL). The resulting suspension was stirred on an ice bath for 10 min and filtered. The cake was rinsed with sat. aq. solution of NaHCO₃ (100 mL) and water (50 mL) and dried under vacuum at 40° C. for 24 h. The crude product was purified by flash chromatography (DCM to 50% of the mixture DCM/MeOH/NH₄OH 90/9/1.5 in DCM) to afford the 21,23-acetonide-rifabutin as a purple solid (37.68 g, 90% yield).

LC/MS A (ESI+): tr=3.20 min, m/z [M+H]⁺=887.48, UV purity: 97% (254 nm).

¹H NMR (300 MHz, CDCl₃): δ 0.39 (d, J=7.0 Hz, 3H), 0.68 (d, J=7.0 Hz, 3H), 0.77-0.88 (m, 9H), 0.89-0.98 (m, 6H), 1.23 (s, 3H), 1.36-1.55 (m, 2H), 1.71-2.18 (m, 6H), 1.77 (s, 3H), 1.96 (s, 3H), 2.04 (s, 3H), 2.22-2.35 (m, 3H), 2.31 (s, 3H), 2.52-2.72 (m, 2H), 2.79 (s, 3H), 2.91-3.09 (m, 2H), 3.06 (dd, J=10.3, 5.4 Hz, 1H), 3.34 (d, J=6.7 Hz, 1H), 3.58 (dd, J=3.1 Hz, 1H), 4.91 (d, J=7.5 Hz, 1H), 5.08 (dd, J=12.1, 6.7 Hz, 1H), 5.87 (d, J=12.2 Hz, 1H), 6.08 (dd, J=15.5, 6.8 Hz, 1H), 6.16 (dd, J=10.7, 1.4 Hz, 1H), 6.28 (dd, J=15.5, 10.7 Hz, 1H), 7.75 (s, 1H), 8.75 (s, 1H), 14.82 (s, 1H).

¹³C NMR (75 MHz, CDCl₃): δ 7.89, 9.11, 9.99, 12.8, 18.0, 20.2, 20.3, 20.9, 23.5, 25.8, 34.1, 35.4, 36.0, 36.8, 40.8, 41.4, 51.5, 56.2, 66.3, 71.2, 74.6, 76.9, 78.9, 95.0, 100.1, 104.6, 106.1, 108.7, 111.6, 113.7, 115.3, 123.8, 125.5, 131.2, 132.6, 140.8, 140.9, 142.3, 155.2, 168.2, 168.9, 170.5, 172.2, 181.3, 192.7.

21,23-Acetonide-rifabutin (10.6 g, 11.9 mmol) was dissolved in dry diethyl ether (500 mL). The solution was cooled to −10° C. with Ar bubbling. After 15 min, a solution of NaOMe (30 mL, 25 w % in MeOH) was slowly added. Precipitation occurred and another portion of diethyl ether (100 mL) was added to homogenize. NaOMe solution was again added (27.4 mL). The solution was stirred at −5° C. for 10 min before the ice bath was removed. The reaction mixture was stirred at rt for 6 h. Sat. aq. solution of NaHCO₃ (400 mL) was added and the layers were separated. The aqueous layer was extracted with diethyl ether (400 mL) and the combined organic layers were washed with brine (300 mL) and evaporated to afford the desired crude product as a black-purple powder. The product was purified by flash chromatography (DCM to 50% of the mixture DCM/MeOH/NH₄OH 90/9/1.5 in DCM). After evaporation, the product was redissolved in acetone and slowly added into water under vigorous stirring. The solid was collected by filtration and dried at 40° C. to yield 8.54 g of the Intermediate I-1.

LC/MS A (ESI+): tr=2.98 min, m/z [M+H]⁺=845.59, UV purity: 97% (254 nm).

¹H NMR (300 MHz, CDCl₃): δ 0.48 (d, J=7.0 Hz, 3H), 0.70 (d, J=6.8 Hz, 3H), 0.74-0.84 (m, 9H), 0.86-0.96 (m, 6H), 1.04 (s, 3H), 1.23-1.33 (m, 1H), 1.43-1.66 (m, 2H), 1.72 (s, 3H), 1.75-2.11 (m, 5H), 2.00 (s, 3H), 2.18-2.29 (m, 3H), 2.21 (s, 3H), 2.47-2.71 (m, 2H), 2.85-3.09 (m, 2H), 3.05-3.14 (m, 1H), 3.10 (s, 3H), 3.27-3.35 (m, 1H), 3.38-3.51 (m, 2H), 3.55 (dd, J=9.2, 3.3 Hz, 1H), 4.93 (dd, J=12.5, 9.5 Hz, 1H), 5.93 (dd, J=15.5, 6.5 Hz, 1H), 6.05-6.16 (m, 2H), 6.24 (dd, J=15.5, 11.7 Hz, 1H), 7.72 (s, 1H), 8.63 (s, 1H), 14.83 (s, 1H).

¹³C NMR (75 MHz, CDCl₃): δ 7.7, 8.4, 12.9, 17.8, 19.9, 20.0, 20.9, 21.0, 24.2, 25.5, 25.8, 34.6, 35.2, 36.3, 39.7, 41.1, 51.4, 51.7, 56.2, 66.3, 71.0, 71.5, 75.5, 83.0, 95.0, 99.6, 104.9, 105.7, 108.6, 111.7, 111.8, 114.3, 124.1, 125.5, 132.0, 132.4, 140.3, 142.6, 142.7, 155.2, 168.2, 169.1, 171.3, 181.6, 191.2.

### Example 2

### Synthesis of 21,23-acetonide-25-bromoacetate-rifabutin (I-2)

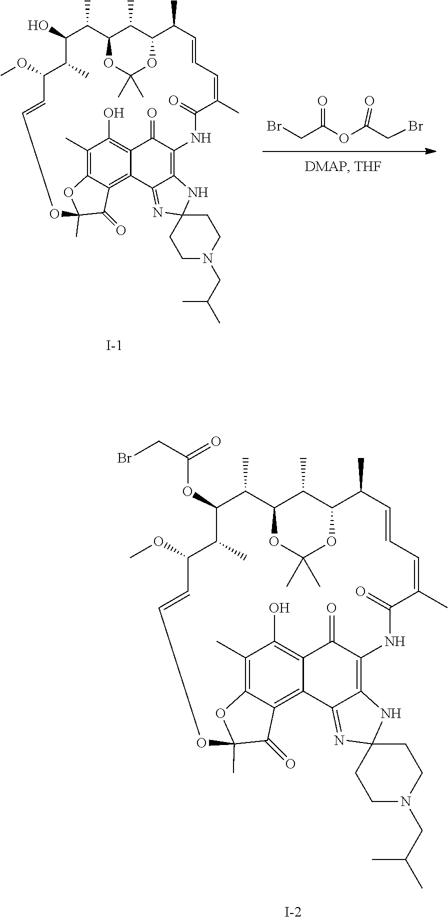

Intermediate I-1 (800 mg, 0.94 mmol, 1 eq) was dissolved in DCM (20 mL) and cold at 0° C. Bromoacetic anhydride (1.54 g, 5.92 mmol, 5 eq) and DMAP (723 mg, 5.92 mmol, 5 eq) were then added and reaction was stirred at 0° C. After 1 h, the mixture was washed with an aqueous solution of HCl-1N (20 mL), a saturated aqueous solution of NaHCO₃ (20 mL) and brine (20 mL). Organic layer was dried over MgSO₄ and concentrated under vacuum. The obtained solid was purified by flash chromatography (CHCl₃/MeOH, from 100/0 to 95/5). Pure fractions were gathered, and solvent evaporated to yield I-2 as a black solid (m=620 mg, yield=54%).

LC/MS A (ESI+): tr=3.48 min, m/z [M+H]⁺=965.48/967.47, UV purity: 95% (254 nm).

¹H NMR (300 MHz, CD₂Cl₂): δ 0.39 (d, J=7.0 Hz, 3H), 0.73 (d, J=7.1 Hz, 3H), 0.79-0.88 (m, 9H), 0.92-0.98 (m, 6H), 1.17 (s, 3H), 1.47-1.60 (m 2H), 1.74 (s, 3H), 1.78-2.07 (m, 6H), 2.03 (d, J=0.8 Hz, 3H), 2.20-2.35 (m, 3H), 2.28 (s, 3H), 2.58-2.76 (m, 2H), 2.83 (s, 3H), 2.88-3.05 (m, 2H), 3.01 (dd, J=10.3, 5.2 Hz, 1H), 3.34-3.41 (m, 1H), 3.59 (dd, J=10.6, 3.2 Hz, 1H), 3.74 (d, J=12.3 Hz, 1H), 3.79 (d, J=12.3 Hz, 1H), 4.96-5.01 (m, 1H), 5.03 (dd, J=12.1, 6.9 Hz, 1H), 5.93 (dd, J=12.1, 0.9 Hz, 1H), 6.05 (dd, J=15.6, 6.9 Hz, 1H), 6.16 (dd, J=10.7, 1.4 Hz, 1H), 6.29 (dd, J=15.6, 10.7 Hz, 1H), 7.75 (s, 1H), 8.77 (s, 1H), 14.87 (s, 1H).

¹³C NMR (75 MHz, CD₂C₂): δ 7.8, 9.7, 9.9, 12.9, 17.8, 20.1, 20.3, 20.9, 21.0, 24.0, 25.9, 26.1, 26.7, 34.7, 35.6, 36.3, 36.4, 40.8, 41.0, 51.7, 51.8, 56.3, 66.6, 71.1, 76.6, 77.0, 79.2, 95.1, 100.1, 104.9, 106.1, 108.9, 112.0, 114.0, 114.5, 124.1, 125.8, 131.8, 132.5, 140.9, 141.5, 142.6, 155.5, 166.7, 168.5, 169.0, 172.3, 181.7, 192.5.

### Example 3

### Synthesis of 21,23-acetonide-25-azidoacetate-rifabutin (I-3)

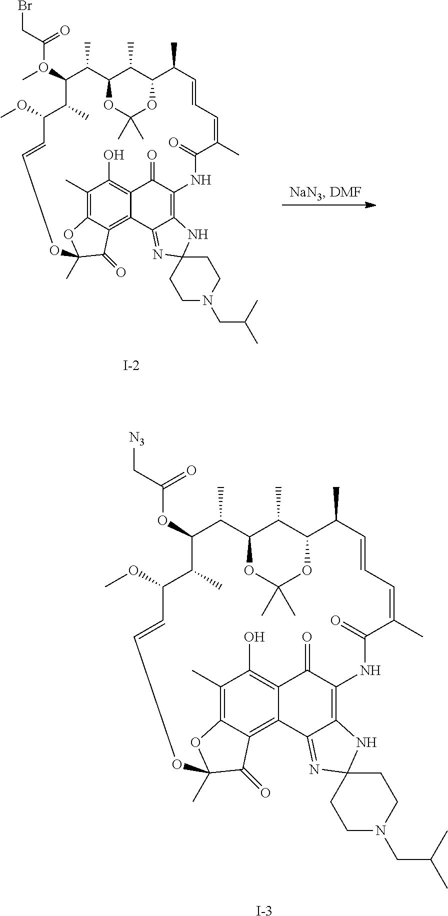

To a solution of I-2 (2.00 g, 2.07 mmol, 1 eq) in DMF (30 mL) was added sodium azide (141 mg, 2.17 mmol, 1.05 eq) and reaction was stirred at rt. After 3 h, mixture was concentrated under vacuum. The obtained oil was dissolved in EtOAc (50 mL) and washed with a saturated aqueous solution of NaHCO₃ (50 mL) and brine (50 mL). The organic layer was dried over MgSO₄ and concentrated under vacuum. The obtained solid was purified by flash chromatography (cyclohexane/acetone/TEA from 100/0/0 to 80/18.5/1.5) to yield the desired product I-3 (m=880 mg, yield=50%).

LC/MS A (ESI+): tr=2.57, m/z [M+H]⁺=928.48, UV purity: 99% (254 nm).

¹H NMR (300 MHz, CDCl₃): δ 0.48 (d, J=6.9 Hz, 3H), 0.73 (d, J=7.1 Hz, 3H), 0.78-0.88 (m, 9H), 0.88-0.96 (m, 6H), 1.20 (s, 3H), 1.45-1.54 (m, 1H), 1.54-1.69 (m, 1H), 1.73-1.92 (m, 4H), 1.76 (s, 3H), 1.99-2.20 (m, 2H), 2.01 (s, 3H), 2.22-2.36 (m, 3H), 2.31 (s, 3H), 2.47-2.67 (m, 2H), 2.81 (s, 3H), 2.87-3.08 (m, 3H), 3.30 (dd, J=7.5, 1.5 Hz, 1H), 3.57 (dd, J=10.7, 3.2 Hz, 1H), 3.70 (d, J=16.9 Hz, 1H), 3.78 (d, J=16.9 Hz, 1H), 4.99-5.15 (m, 2H), 5.92 (d, J=12.3 Hz, 1H), 6.06 (dd, J=15.6, 6.9 Hz, 1H), 6.15 (dd, J=10.6, 1.4 Hz, 1H), 6.26 (dd, J=15.6, 10.6 Hz, 1H), 7.75 (s, 1H), 8.75 (brs, 1H), 14.81 (s, 1H).

¹³C NMR (75 MHz, CDCl₃): δ 7.8, 9.7, 9.8, 12.9, 18.0, 20.14, 20.18, 20.93, 20.94, 23.45, 25.7, 25.9, 34.2, 35.5, 36.2, 36.8, 40.5, 40.9, 50.5, 51.50, 51.59, 56.0, 66.4, 71.0, 76.1, 76.4, 79.9, 95.2, 100.1, 104.6, 106.0, 108.7, 111.6, 113.8, 113.9, 123.8, 125.4, 131.4, 132.5, 140.5, 141.6, 142.3, 155.1, 167.6, 168.2, 168.8, 172.0, 181.3, 192.3.

### Example 4

### General Procedure for the Preparation of Triazole Derivatives

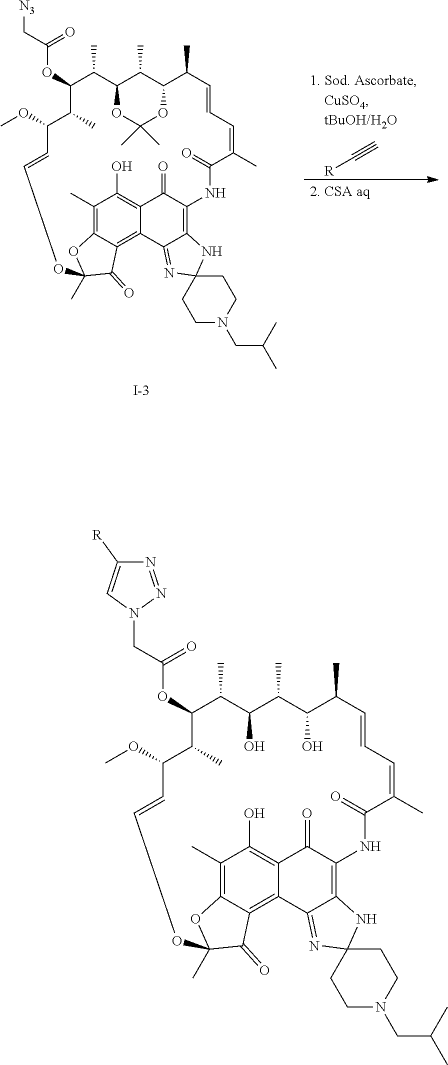

A solution of 21,23-acetonide-25-azidoacetate-rifabutin I-3 (0.05 mol/L) with sodium ascorbate (0.007 mol/L) and CuSO₄ 5H₂O (0.007 mol/L) was prepared in tBuOH/H₂O (3/1). The desired alkyne (0.32 mmol, 1.5 eq) was packed in Kimble reactor, then 400 μL of solution of 25-azido-acetic-21,23-acetonide-rifabutin in tBuOH/H₂O (corresponding to 20 mg, 0.021 mmol, 1 eq of 25-azido-acetic-21,23-acetonide-rifabutin, 0.6 mg, 0.003 mmol, 0.15 eq of sodium ascorbate and 0.8 mg, 0.003 mmol, 0.15 eq of CuSO₄ 5H₂O) was added. Reaction was stirred at 60° C.

After 24 h, 400 μL of a solution of camphor sulfonic acid in water (0.5N) was added to the mixture. Reaction was stirred at rt for 24 h. The mixture was then deposited on ColumnPoraPak (Rxn RP 6CC) equilibrated with water. Elution was processed by 5 mL of water, 5 mL of a saturated aqueous solution of NaHCO₃, 5 mL of a solution of water/ACN (90/10) and 10 mL of ACN. The 10 mL of ACN were collected, frozen and freeze-dried to give the desired product as a purple powder.

Table 5 below summarizes exemplary compounds of the invention that were prepared according to the protocols of Example 4 above. (Data given in Table 5 were obtained following the analytic method B).

TABLE 5
Compounds of the Invention.

| Cpd | Structure | MW | Rt | [M + H]+ |
|---|---|---|---|---|
| 1 | 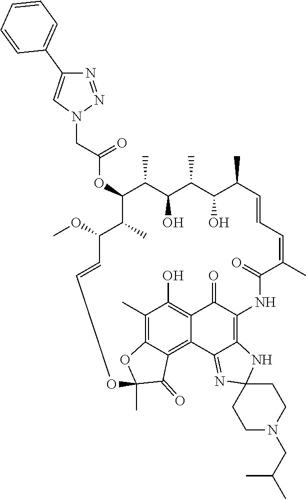 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(-c3ccccc3)nn2)[C@@H]1C)C4=O] | 989.48 | 17.20 | 990.52 |
| 2 | 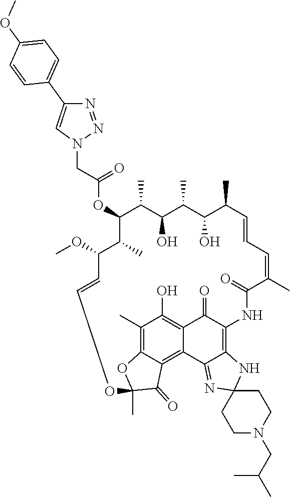 [SMILES: COc1ccc(-c2cn(CC(=O)O[C@H]3[C@H](C)[C@H](O)[C@H](C)[C@@H](O)[C@@H](C)/C=C/C=C(/C)C(=O)NC4=C5NC6(CCN(CC(C)C)CC6)N=C5c5c(c(O)c(C)c6c5C(=O)[C@@](C)(O/C=C/[C@H](OC)[C@H]3C)O6)C4=O)nn2)cc1] | 1019.50 | 16.93 | 1020.54 |
| 3 | 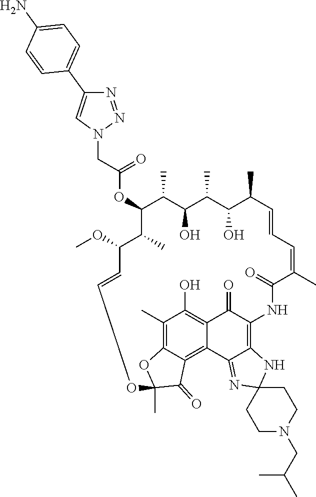 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(-c3ccc(N)cc3)nn2)[C@@H]1C)C4=O] | 1004.50 | 15.19 | 1005.55 |
| 4 | 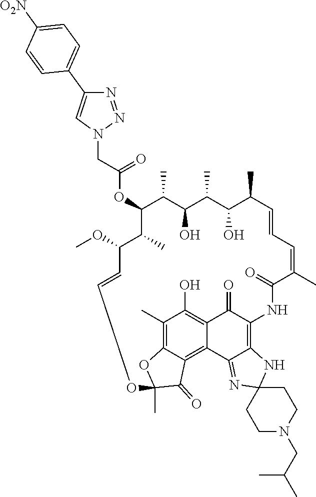 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(-c3ccc([N+](=O)[O-])cc3)nn2)[C@@H]1C)C4=O] | 1034.47 | 17.50 | 1035.54 |
| 5 | 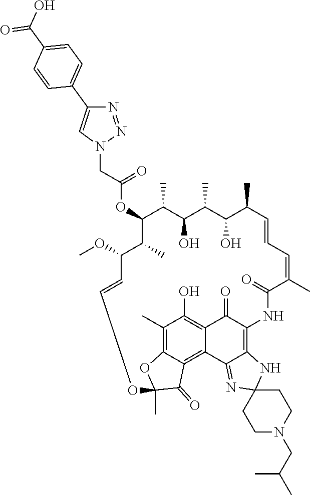 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(-c3ccc(C(=O)O)cc3)nn2)[C@@H]1C)C4=O] | 1033.47 | 12.02 | 1034.51 |
| 6 | 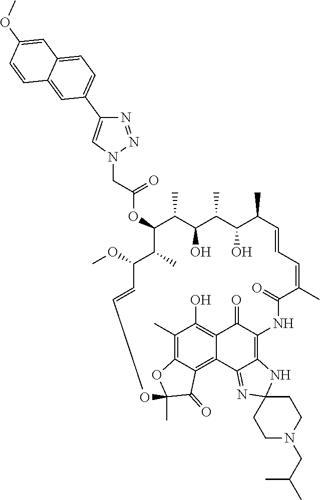 [SMILES: COc1ccc2cc(-c3cn(CC(=O)O[C@H]4[C@H](C)[C@H](O)[C@H](C)[C@@H](O)[C@@H](C)/C=C/C=C(/C)C(=O)NC5=C6NC7(CCN(CC(C)C)CC7)N=C6c6c(c(O)c(C)c7c6C(=O)[C@@](C)(O/C=C/[C@H](OC)[C@H]4C)O7)C5=O)nn3)ccc2c1] | 1069.51 | 18.20 | 1070.55 |
| 7 | 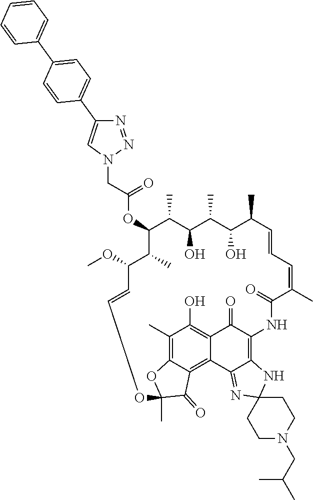 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(-c3ccc(-c5ccccc5)cc3)nn2)[C@@H]1C)C4=O] | 1065.52 | 19.25 | 1066.59 |
| 8 | 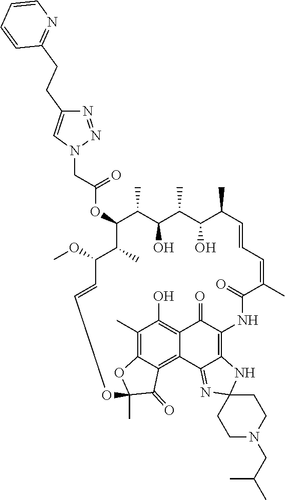 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CCc3ccccn3)nn2)[C@@H]1C)C4=O] | 990.48 | 15.70 | 991.54 |
| 9 | 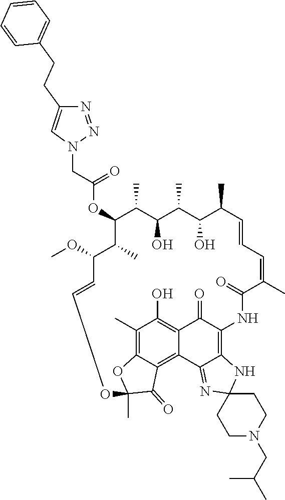 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CCc3ccccc3)nn2)[C@@H]1C)C4=O] | 1017.52 | 17.67 | 1018.60 |
| 10 | 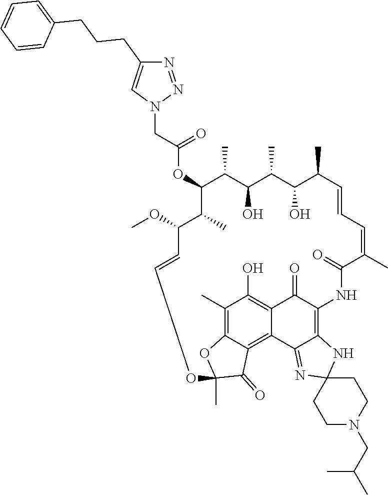 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CCCc3ccccc3)nn2)[C@@H]1C)C4=O] | 1031.53 | 18.20 | 1032.64 |
| 11 | 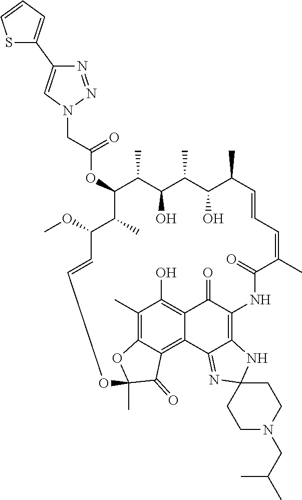 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(-c3cccs3)nn2)[C@@H]1C)C4=O] | 995.44 | 16.87 | 996.53 |
| 12 | 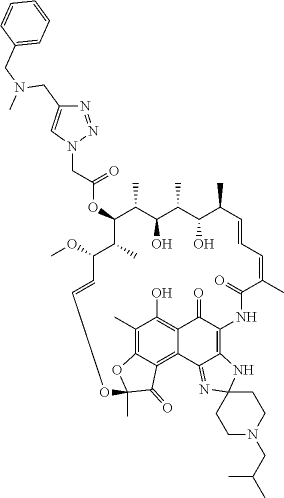 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CN(C)Cc3ccccc3)nn2)[C@@H]1C)C4=O] | 1046.54 | 17.05 | 1047.60 |
| 13 | 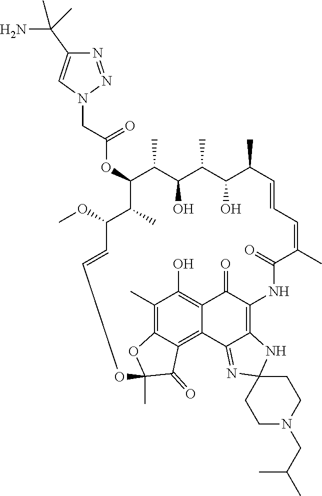 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(C(C)(C)N)nn2)[C@@H]1C)C4=O] | 970.51 | 13.80 | 971.60 |
| 14 | 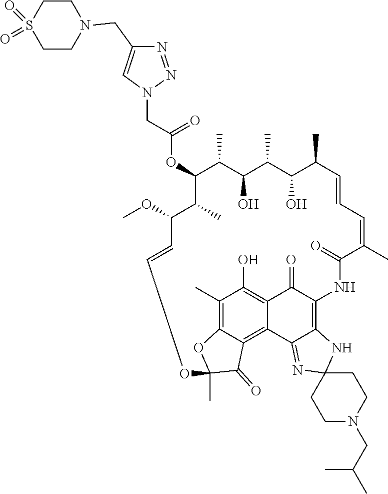 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CN3CCS(=O)(=O)CC3)nn2)[C@@H]1C)C4=O] | 1060.49 | 14.07 | 1061.57 |
| 15 | 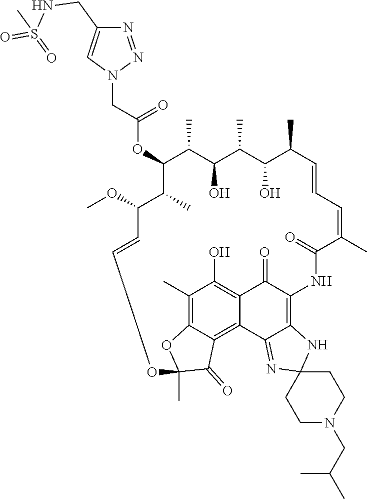 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CNS(C)(=O)=O)nn2)[C@@H]1C)C4=O] | 1020.46 | 14.32 | 1021.53 |
| 16 | 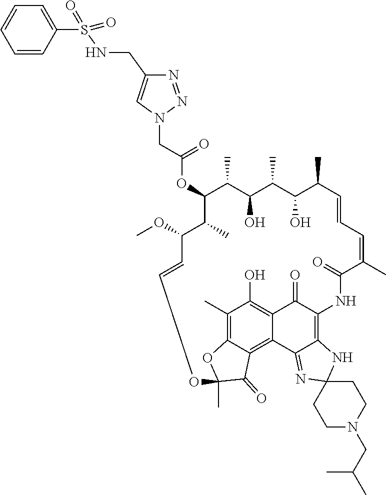 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CNS(=O)(=O)c3ccccc3)nn2)[C@@H]1C)C4=O] | 1082.47 | 15.92 | 1083.53 |
| 17 | 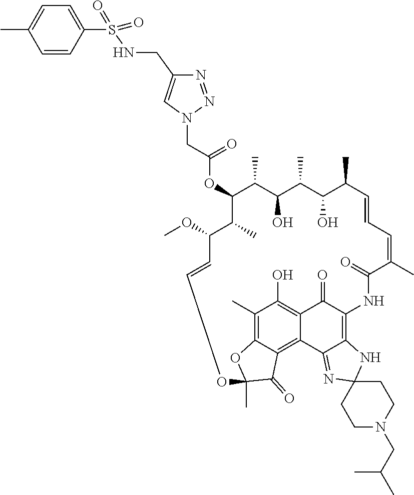 | 1096.49 | 16.45 | 1097.57 |
| 18 | 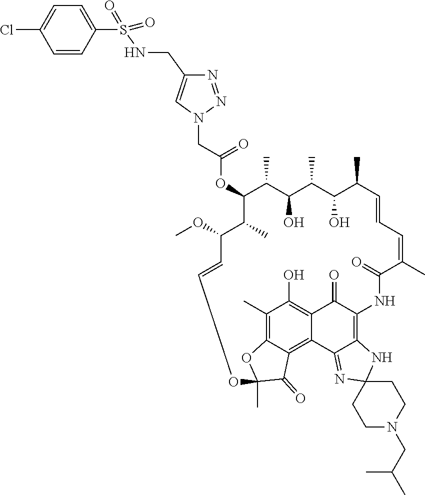 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CNS(=O)(=O)c3ccc(Cl)cc3)nn2)[C@@H]1C)C4=O] | 1116.43 | 16.87 | 1117.48 |
| 19 | 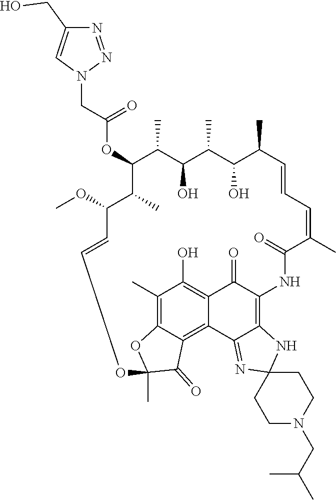 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CO)nn2)[C@@H]1C)C4=O] | 943.46 | 13.62 | 944.54 |
| 20 | 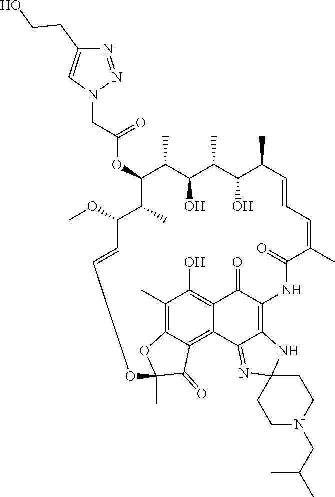 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CCO)nn2)[C@@H]1C)C4=O] | 957.48 | 13.60 | 958.55 |
| 21 | 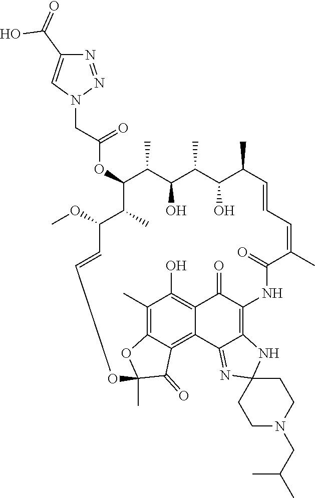 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(C(=O)O)nn2)[C@@H]1C)C4=O] | 957.44 | 11.30 | 958.51 |
| 22 | 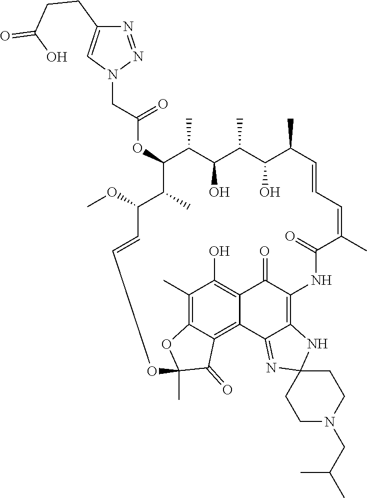 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CCC(=O)O)nn2)[C@@H]1C)C4=O] | 985.47 | 11.28 | 986.56 |
| 23 | 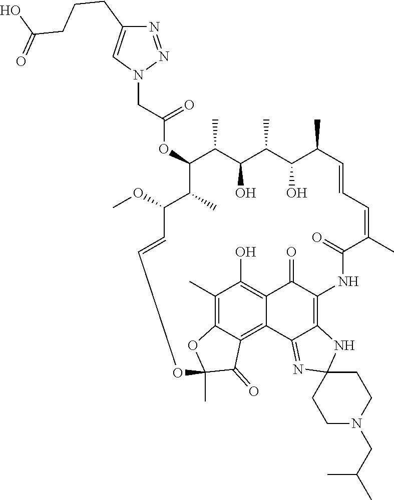 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CCCC(=O)O)nn2)[C@@H]1C)C4=O] | 999.49 | 11.30 | 1000.56 |
| 24 | 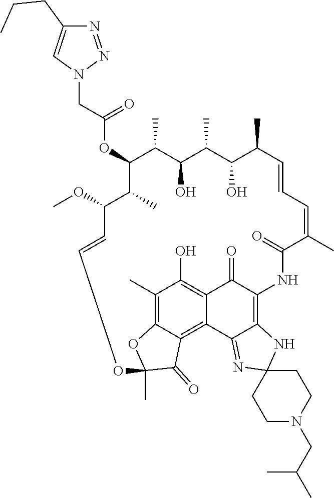 [SMILES: CCCc1cn(CC(=O)O[C@H]2[C@H](C)[C@H](O)[C@H](C)[C@@H](O)[C@@H](C)/C=C/C=C(/C)C(=O)NC3=C4NC5(CCN(CC(C)C)CC5)N=C4c4c(c(O)c(C)c5c4C(=O)[C@@](C)(O/C=C/[C@H](OC)[C@H]2C)O5)C3=O)nn1] | 955.50 | 16.25 | 956.57 |
| 25 | 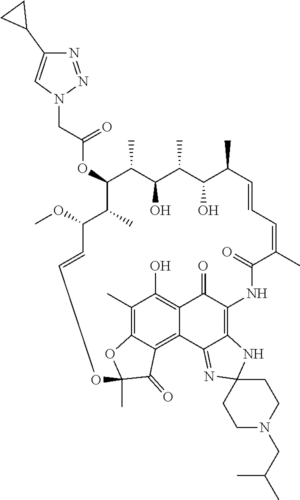 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(C3CC3)nn2)[C@@H]1C)C4=O] | 953.48 | 15.85 | 954.59 |
| 26 | 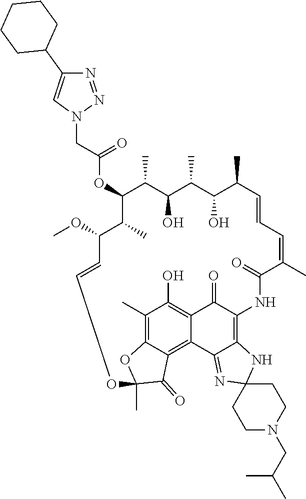 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(C3CCCCC3)nn2)[C@@H]1C)C4=O] | 995.53 | 17.89 | 996.60 |
| 27 | 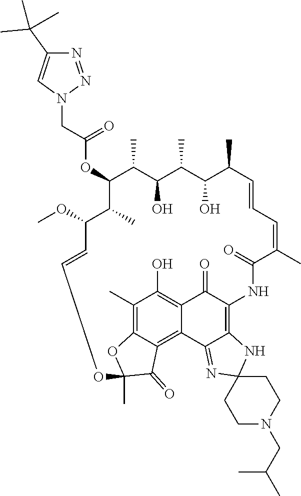 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(C(C)(C)C)nn2)[C@@H]1C)C4=O] | 969.52 | 16.75 | 970.61 |
| 28 | 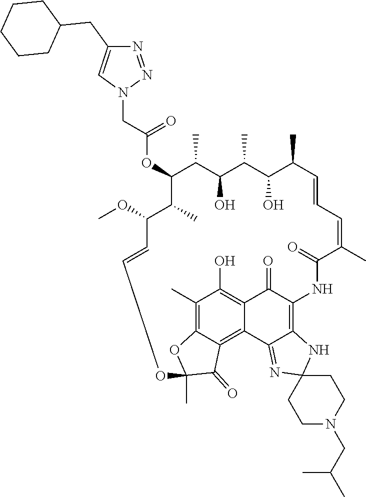 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CC3CCCCC3)nn2)[C@@H]1C)C4=O] | 1009.55 | 18.67 | 1010.64 |

### Additional Analytical Data (NMR):

### Compound 1:

¹H NMR (300 MHz, CDCl3): δ (ppm) −0.14 (d, J=7.2 Hz, 3H), 0.52 (d, J=6.8 Hz, 3H), 0.80 (d, J=6.8 Hz, 3H), 0.93 (d, J=6.8 Hz, 6H), 1.04 (d, J=6.8 Hz, 3H), 1.18-1.26 (in, 1H), δ 1.67-1.76 (m, 1H), 1.71 (s, 3H), 1.77-1.93 (m, 3H), 1.94-2.09 (m, 3H), 2.03 (s, 3H), 2.24-2.34 (m, 2H), 2.31 (s, 3H), 2.37-2.47 (m, 1H), 2.55-2.72 (m, 2H), 2.88-3.07 (m, 4H), 3.09 (s, 3H), 3.20 (dd, J=9.5 Hz, 3.5 Hz, 1H), 3.36 (d, J=8.5 Hz, 1H), 3.64 (d, J=9.8 Hz, 1H), 4.88 (d, J=10.8 Hz, 1H), 4.92-5.16 (m, 2H), 5.41 (dd, J=12.4 Hz, 9.9 Hz, 1H), 5.81-5.98 (m, 1H), 6.12-6.30 (m, 3H), 7.28-7.36 (m, 1H), 7.41 (t, J=7.5 Hz, 2H), 7.82 (d, J=7.8 Hz, 2H), 7.95 (s, 1H), 8.22 (s, 1H), 9.60 (s, 1H), 14.43 (s, 1H).

¹³C NMR (75 MHz, CDCl3): δ (ppm) 7.85, 8.95, 11.06, 12.61, 17.40, 20.32, 21.10, 21.14, 22.69, 26.12, 32.96, 35.43, 36.39, 37.32, 38.48, 39.46, 51.59, 51.73, 56.58, 66.56, 71.85, 74.32, 76.37, 84.03, 95.12, 104.39, 107.98, 109.72, 111.54, 115.34, 116.37, 121.60, 123.37, 124.98, 126.06, 128.39, 129.09, 130.87, 132.93, 132.99, 140.87, 141.96, 146.69, 148.23, 155.05, 166.06, 168.02, 168.63, 171.68, 180.63, 192.61.

### Compound 8:

¹H NMR (300 MHz, CDCl3): δ (ppm) −0.13 (d, J=7.1 Hz, 3H), 0.52 (d, J=6.9 Hz, 3H), 0.81 (d, J=6.9 Hz, 3H), 0.94 (d, J=6.5 Hz, 6H), 1.06 (d, J=6.5 Hz, 3H), 1.18-1.31 (m, 5H), 1.68-1.74 (m, 1H), 1.72 (s, 3H), 1.81-2.04 (m, 6H), 2.03 (s, 3H), 2.24-2.34 (m, 2H), 2.32 (s, 3H), 2.35-2.46 (m, 1H), 2.56-2.73 (m, 2H), 2.88-3.07 (m, 4H), 3.11 (s, 3H), 3.21 (dd, J=9.6 Hz, 3.5 Hz, 1H), 3.28 (d, J=8.3 Hz, 1H), 3.65 (d, J=10.2 Hz, 1H), 4.89 (dd, J=10.8 Hz, 1.3 Hz, 1H), 4.99-5.15 (m, 2H), 5.41 (dd, J=12.7 Hz, 9.6 Hz, 1H), 5.85-5.99 (m, 1H), 6.18-6.30 (m, 3H), 7.22 (ddd, J=7.5 Hz, 4.9 Hz, 1.1 Hz, 1H), 7.76 (td, J=7.7 Hz, 1.7 Hz, 1H), 8.15 (d, J=7.9 Hz, 1H), 8.21-8.28 (m, 2H), 8.56-8.62 (m, 1H), 9.57 (s, 1H), 14.46 (s, 1H).

¹³C NMR (75 MHz, CDCl3): δ (ppm) 7.91, 8.98, 11.13, 12.58, 17.47, 20.39, 21.14, 21.18, 22.72, 26.18, 30.03, 33.06, 35.54, 36.47, 37.41, 38.49, 39.44, 51.71, 51.78, 56.75, 66.62, 71.98, 74.58, 76.47, 83.93, 95.12, 104.49, 108.00, 109.75, 111.65, 115.43, 116.33, 120.64, 123.16, 123.53, 124.02, 125.06, 132.90, 133.02, 137.16, 140.97, 142.01, 146.61, 148.92, 149.78, 150.55, 155.18, 166.03, 168.14, 168.68, 171.76, 180.80, 192.75.

### Compound 12:

¹H NMR (300 MHz, CDCl3): δ (ppm) −0.17, (d, J=7.0 Hz, 3H), 0.46 (d, J=6.8 Hz, 3H), 0.79 (d, J=7.3 Hz, 3H), 0.93 (d, J=6.5 Hz, 6H), 1.03 (d, J=6.9 Hz, 3H), 1.12-1.27 (m, 1H), 1.63-1.74 (m, 1H), 1.71 (s, 3H), 1.77-2.01 (m, 6H), 2.03 (s, 3H), 2.22 (s, 3H), 2.25-2.31 (m, 2H), 2.29 (s, 3H), 2.36-2.46 (m, 1H), 2.55-2.71 (m, 2H), 2.85-3.01 (m, 4H), 3.07 (s, 3H), 3.16 (dd, J=9.6 Hz, 3.3 Hz, 1H), 3.32 (d, J=8.3 Hz, 1H), 3.44-3.58 (m, 2H), 3.62 (d, J=9.9 Hz, 1H), 3.72 (s, 2H), 4.84 (d, J=10.7 Hz, 1H), 4.88-5.09 (m, 2H), 5.38 (dd, J=12.6 Hz, 9.7 Hz, 1H), 5.82-5.97 (m, 1H), 6.15-6.29 (m, 3H), 7.21-7.33 (m, 5H), 7.63 (s, 1H), 8.22 (s, 1H), 9.57 (s, 1H), 14.44 (s, 1H).

¹³C NMR (75 MHz, CDCl3): δ (ppm) 7.82, 8.92, 11.09, 12.54, 17.40, 20.34, 21.10, 21.14, 22.69, 26.13, 32.96, 35.47, 36.40, 37.31, 38.39, 39.40, 42.38, 51.49, 51.73, 52.29, 56.55, 61.39, 66.58, 71.90, 74.30, 76.37, 83.93, 95.10, 104.43, 107.98, 109.68, 111.59, 115.29, 116.18, 123.49, 124.49, 125.00, 127.28, 128.53, 129.22, 132.87, 132.94, 139.06, 140.85, 141.97, 145.67, 146.66, 155.07, 166.16, 168.07, 168.62, 171.64, 180.69, 192.59.

### Example 5

### Antibacterial Activity

MIC values were determined by broth microdilution method according to the CLSI guideline. Unless otherwise mentioned, MIC against A. baumannii was performed in RPMI medium supplemented with 10% FCS. MIC against M. abscessus was performed in Middlebrook 7H9 broth supplemented with Middlebrook ADC growth supplement (10%). All other MIC were performed in standard cation adjusted Mueller Hinton broth, supplemented with Middlebrook ADC growth supplement (5%) for the slow growing mycobacteria (M. xenopi, M. kansasii and M. avium).

The following procedure applies to all species tested, except for M. xenopi. From a fresh culture plate (from overnight to several days depending on the bacteria), cells were resuspended in 0.9% (w/v) saline solution and bacterial inoculum was prepared in the respective testing medium with 5×10⁵ CFU/mL. For M. xenopi, the inoculum was prepared from a fresh liquid culture (7H9+ADC). The appropriate volumes of a 10 mg/mL compound solution were directly dispensed in the 96-well assay plate using a digital dispenser to create two-fold dilution series from 32 to 0.002 μg/mL final concentration for A. baumannii and S. aureus; from 16 to 0.016 μg/mL for M. abscessus and M. avium; and from 1 to 6×10⁻⁵ μg/mL for M. kansasii and M. xenopi. 100 μL of bacterial suspension was finally added to the compounds. Plates were covered and incubated without shaking at 35° C. for 20 hours for A. baumanii and S. aureus; at 30° C. for 4 days for M. abscessus; and at 37° C. for 7 days (at least) for M. kansasii (covered with aluminum foil to avoid light exposure), M. avium and M. xenopi. Every experiment contained an antibiotic as quality control. MIC was determined visually as the lowest concentration of a compound that prevents visible growth of the bacteria.

The in vitro activity of compounds described herein was determined against S. aureus (UAMS-1625 strain), A. baumannii (HUMC1 strain), M. abscessus (ATCC 19977), M. kansasii (ATCC12478), M. avium (ATCC25291), and M. xenopi (ATCC19250). The MIC values, given in μg/mL, are given in Table 6, below.

TABLE 6
In vitro activity (μg/mL) of described compounds against A. baumannii,
S. aureus, M. abscessus, M. kansasii, M. xenopi and M. avium.

| Com- | | | | | | |
|---|---|---|---|---|---|---|
| pound | S. | A. | M. | M. | M. | M. |
| Number | aureus | baumannii | abscessus | kansasii | xenopi | avium |
| Rifabutin | 0.016 | ≤0.002 | 8 | 0.001 | 0.03 | 0.125 |
| 1 | 0.125 | 0.004 | 0.25 | 0.002 | 0.016 | 0.03 |
| 2 | 0.125 | 0.016 | 0.25 | 0.002 | 0.125 | 0.06 |
| 3 | 0.03 | ≤0.002 | 1 | | | |
| 4 | 0.5 | 0.125 | 0.125 | 0.002 | 0.03 | 0.03 |
| 6 | 8 | 1 | 0.25 | 0.002 | 0.03 | 0.06 |
| 7 | 8 | 1 | 1 | | | |
| 8 | 0.03 | ≤0.002 | 0.25 | 0.004 | 0.06 | 0.06 |
| 9 | 0.5 | 0.03 | 0.25 | 0.002 | 0.03 | 0.06 |
| 10 | 8 | 0.125 | 0.5 | | | |
| 11 | 0.03 | 0.008 | 1 | | | |
| 12 | 0.125 | 0.008 | 0.25 | 0.004 | 0.03 | 0.03 |
| 13 | 0.125 | ≤0.002 | 1 | | | |
| 14 | 0.06 | ≤0.002 | 1 | | | |
| 15 | 0.125 | ≤0.002 | 1 | | | |
| 16 | 0.125 | 0.008 | 0.5 | | | |
| 17 | 0.25 | 0.008 | 0.5 | | | |
| 18 | 0.5 | 0.06 | 0.5 | | | |
| 24 | 0.03 | 0.004 | 1 | | | |
| 25 | 0.03 | ≤0.002 | 4 | | | |
| 26 | 0.125 | 0.016 | 0.25 | 0.002 | 0.03 | 0.03 |
| 27 | 0.06 | 0.004 | 2 | | | |
| 28 | 0.5 | 0.03 | 0.5 | | | |

1. A compound of Formula (I) or a pharmaceutically acceptable salt, solvate, hydrate, tautomer, or enantiomer, thereof: 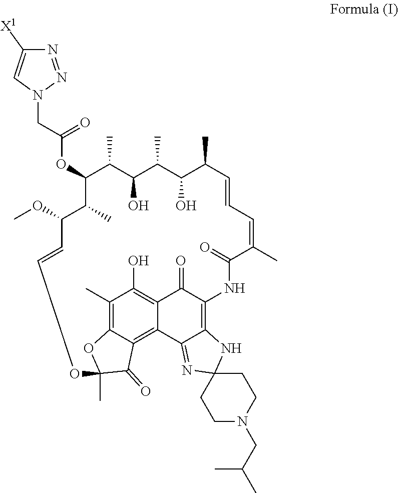
wherein:
X¹ is independently selected from —COOH, —C₁-C₆ alkyl, —C₃-C₈ cycloalkyl, —C₁-C₆ alkylene-(C₃-C₈ cycloalkyl), 5- to 10-membered heterocycloalkyl, —C₁-C₆ alkylene-(5- to 10-membered heterocycloalkyl), —C₆-C₁₀ aryl, —C₁-C₆ alkylene-(C₆-C₁₀ aryl), 5- to 10-membered heteroaryl, and —C₁-C₆ alkylene-(5- to 10-membered heteroaryl);
wherein said alkyl is optionally substituted with one or more R¹;
wherein said cycloalkyl is each independently, at each occurrence, optionally substituted with one or more R²;
wherein said heterocycloalkyl is each independently, at each occurrence, optionally substituted with one or more R³;
wherein said aryl is each independently, at each occurrence, optionally substituted with one or more R⁴;
wherein said heteroaryl is each independently, at each occurrence, optionally substituted with one or more R⁵;
R¹, R², R³, R⁴ and R⁵ are each independently, at each occurrence, selected from —OH, —OC₁-C₆ alkyl, —NR⁶R⁷, —NHSO₂R⁸, —COOH, oxo, —NO₂, phenyl, halogen, and cyano;
R⁶ and R⁷ are each independently, at each occurrence, selected from —H and —C₁-C₆ alkyl, wherein said C₁-C₆ alkyl is optionally substituted with phenyl; and
R⁸ is independently selected from —C₁-C₆ alkyl and phenyl, wherein said phenyl is optionally substituted with —C₁-C₆ alkyl or halogen.

2. The compound of claim 1, wherein:
R¹, R², R³, R⁴ and R⁵ are each independently, at each occurrence, selected from —OH, —OC₁-C₆ alkyl, —NR⁶R⁷, —NHSO₂R⁸, —COOH, oxo, —NO₂, and phenyl;
R⁶ and R⁷ are each independently, at each occurrence, selected from —H and —C₁-C₆ alkyl, wherein said C₁-C₆ alkyl is optionally substituted with phenyl; and
R⁸ is independently selected from —C₁-C₆ alkyl and phenyl, wherein said phenyl is optionally substituted with —C₁-C₆ alkyl or halogen.

3. The compound of claim 1 or 2, wherein X¹ is independently selected from —COOH, —C₁-C₆ alkyl, —C₃-C₆ cycloalkyl, —C₁-C₆ alkylene-(C₃-C₆ cycloalkyl), 5- to 7-membered heterocycloalkyl, —C₁-C₆ alkylene-(5- to 7-membered heterocycloalkyl), —C₆-C₁₀ aryl, —C₁-C₆ alkylene-(C₆-C₁₀ aryl), 5- to 10-membered heteroaryl, or —C₁-C₆ alkylene-(5- to 10-membered heteroaryl);
wherein said alkyl is optionally substituted with one or more R¹;
said cycloalkyl is each independently, at each occurrence, optionally substituted with one or more R²;
said heterocycloalkyl is each independently, at each occurrence, optionally substituted with one or more R³;
said aryl is each independently, at each occurrence, optionally substituted with one or more R⁴;
said 5- to 10-membered heteroaryl is each unsubstituted;
R¹ is independently, at each occurrence, selected from —OH, —NR⁶R⁷, —NHSO₂R⁸, and —COOH;
R² is selected from —NR⁶R⁷;
R³ is oxo; and
R⁴ is independently, at each occurrence, selected from —OC₁-C₆ alkyl, —NR⁶R⁷, —COOH, —NO₂, and phenyl.

4. The compound of any of the preceding claims, wherein X¹ is independently selected from —C₁-C₅ alkyl, —C₅-C₆ cycloalkyl, —C₁-C₂ alkylene-(C₅-C₆ cycloalkyl), 5- to 6-membered heterocycloalkyl, —C₁-C₂ alkylene-(5- to 6-membered heterocycloalkyl), —C₆-C₁₀ aryl, —C₁-C₆ alkylene-(C₆-C₁₀ aryl), and 5- to 6-membered heteroaryl;
wherein said alkyl is optionally substituted with one to three R¹, preferably with exactly one R¹; said cycloalkyl is each unsubstituted; said heterocycloalkyl is each independently, at each occurrence, optionally substituted with one or two R³; said aryl is each independently, at each occurrence, optionally substituted with one to three R⁴, preferably with exactly one R⁴;
R¹ is independently, at each occurrence, selected from —NR⁶R⁷ and —NHSO₂R⁸;
R³ is oxo;
R⁴ is independently, at each occurrence, selected from —OC₁-C₆ alkyl, —NH₂, and —NO₂;
R⁶ and R⁷ are independently, at each occurrence, selected from —H, —CH₃, and —CH₂—C₆H₅; and
R⁸ is —CH₃ or phenyl, wherein said phenyl is optionally substituted with one or more —CH₃ or —C₁.

5. The compound of any of the preceding claims, wherein X¹ is independently selected from —C₁-C₃ alkyl, cyclohexyl, —C₁ alkylene-(cyclohexyl), phenyl, —C₁-C₃ alkylene-(phenyl), and 2-pyridinyl;
wherein said alkyl is optionally substituted with one or two R¹, preferably with exactly one R¹; said cyclohexyl is each unsubstituted; said phenyl each is unsubstituted or substituted with one or two, preferably with exactly one —OCH₃ or —NO₂; said 2-pyridinyl is unsubstituted;
R¹ is independently, at each occurrence, selected from —N(CH₃)(CH₂C₆H₅), and —NHSO₂R⁸; and
R⁸ is phenyl, wherein said phenyl is optionally substituted at the 4-position with —CH₃ or —C₁.

6. The compound of claim 1, wherein X¹ is —C₁-C₆ alkyl, wherein said alkyl is optionally substituted with one or more R¹; and wherein R¹ is independently, at each occurrence, selected from —OH, —OC₁-C₆ alkyl, —NR⁶R⁷, —NHSO₂R, —COOH, oxo, —NO₂, phenyl, halogen, and cyano.

7. The compound of claim 1, wherein X¹ is —C₃-C₈ cycloalkyl or —C₁-C₆ alkylene-(C₃-C₈ cycloalkyl), wherein said cycloalkyl is each independently, at each occurrence, optionally substituted with one or more R²; and wherein R² is selected from —OH, —OC₁-C₆ alkyl, —NR⁶R⁷, —NHSO₂R, —COOH, oxo, —NO₂, phenyl, halogen, and cyano.

8. The compound of claim 1, wherein X¹ is 5- to 10-membered heterocycloalkyl or —C₁-C₆ alkylene-(5- to 10-membered heterocycloalkyl), wherein said heterocycloalkyl is each independently, at each occurrence, optionally substituted with one or more R³; and wherein R³ is selected from —OH, —OC₁-C₆ alkyl, —NR⁶R⁷, —NHSO₂R⁸, —COOH, oxo, —NO₂, phenyl, halogen, and cyano.

9. The compound of claim 1, wherein X¹ is —C₆-C₁₀ aryl or —C₁-C₆ alkylene-(C₆-C₁₀ aryl), wherein said aryl is each independently, at each occurrence, optionally substituted with one or more R⁴; and wherein R⁴ is independently, at each occurrence, selected from —OH, —OC₁-C₆ alkyl, —NR⁶R⁷, —NHSO₂R⁸, —COOH, oxo, —NO₂, phenyl, halogen, and cyano.

10. The compound of claim 1, wherein X¹ is 5- to 10-membered heteroaryl or —C₁-C₆ alkylene-(5- to 10-membered heteroaryl); wherein said heteroaryl is independently, at each occurrence, optionally substituted with one or more R⁵; and wherein R⁵ is selected from —OH, —OC₁-C₆ alkyl, —NR⁶R⁷, —NHSO₂R⁸, —COOH, oxo, —NO₂, phenyl, halogen, and cyano.

11. The compound according to claim 1, wherein the compound is selected from the group consisting of:

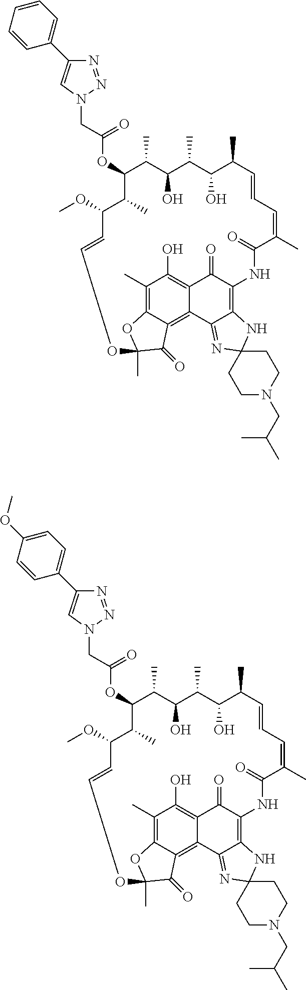 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(-c3ccccc3)nn2)[C@@H]1C)C4=O ; COc1ccc(-c2cn(CC(=O)O[C@H]3[C@H](C)[C@H](O)[C@H](C)[C@@H](O)[C@@H](C)/C=C/C=C(/C)C(=O)NC4=C5NC6(CCN(CC(C)C)CC6)N=C5c5c(c(O)c(C)c6c5C(=O)[C@@](C)(O/C=C/[C@H](OC)[C@H]3C)O6)C4=O)nn2)cc1]

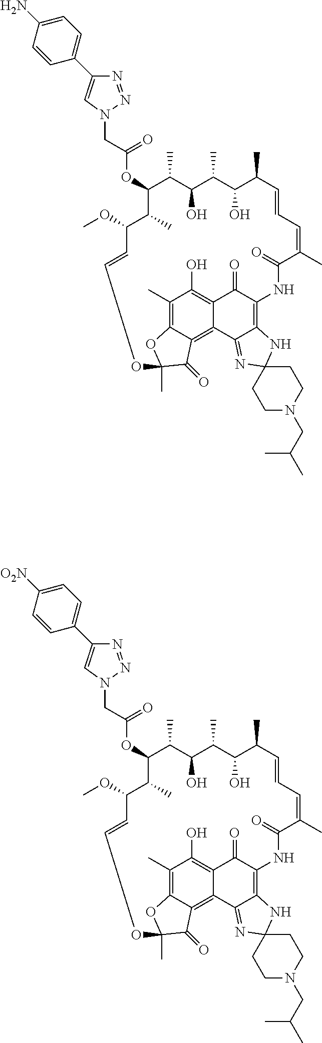 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(-c3ccc(N)cc3)nn2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(-c3ccc([N+](=O)[O-])cc3)nn2)[C@@H]1C)C4=O]

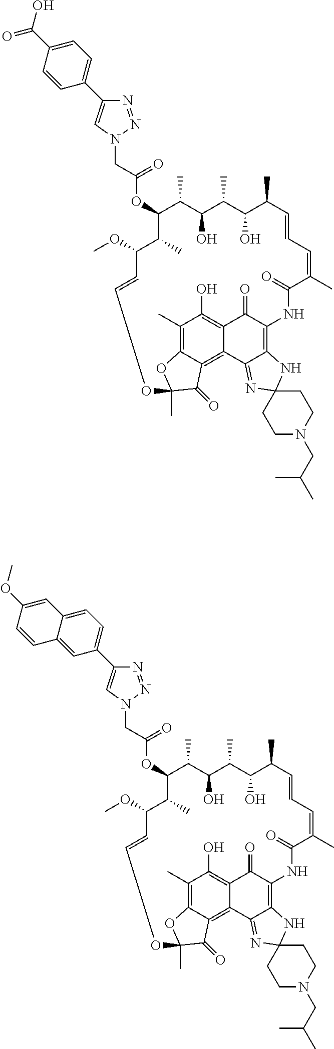 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(-c3ccc(C(=O)O)cc3)nn2)[C@@H]1C)C4=O ; COc1ccc2cc(-c3cn(CC(=O)O[C@H]4[C@H](C)[C@H](O)[C@H](C)[C@@H](O)[C@@H](C)/C=C/C=C(/C)C(=O)NC5=C6NC7(CCN(CC(C)C)CC7)N=C6c6c(c(O)c(C)c7c6C(=O)[C@@](C)(O/C=C/[C@H](OC)[C@H]4C)O7)C5=O)nn3)ccc2c1]

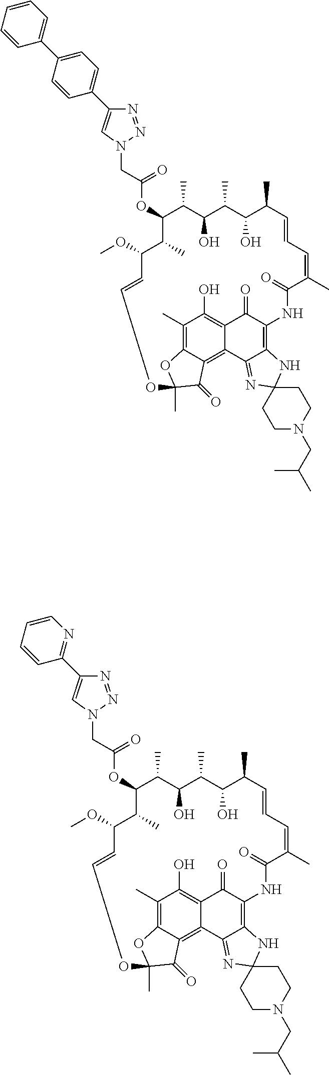 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(-c3ccc(-c5ccccc5)cc3)nn2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(-c3ccccn3)nn2)[C@@H]1C)C4=O]

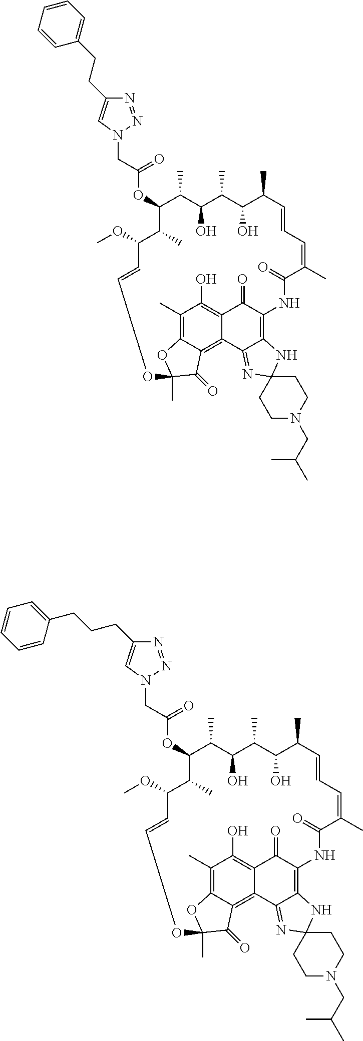 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CCc3ccccc3)nn2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CCCc3ccccc3)nn2)[C@@H]1C)C4=O]

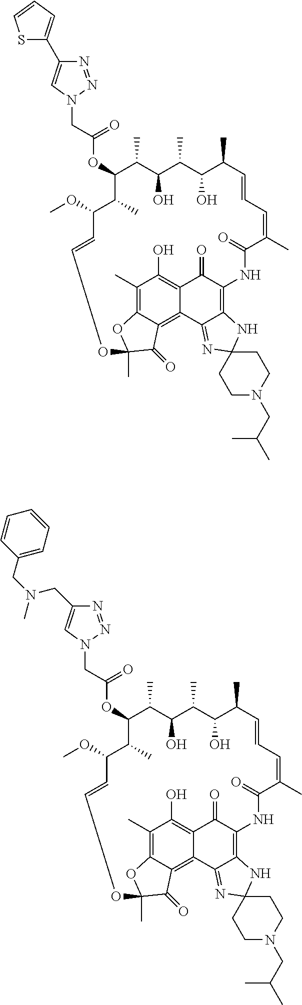 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(-c3cccs3)nn2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CN(C)Cc3ccccc3)nn2)[C@@H]1C)C4=O]

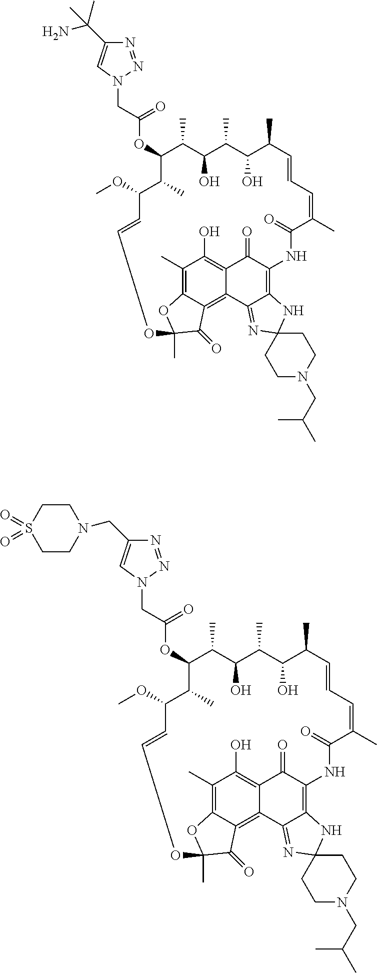 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(C(C)(C)N)nn2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CN3CCS(=O)(=O)CC3)nn2)[C@@H]1C)C4=O]

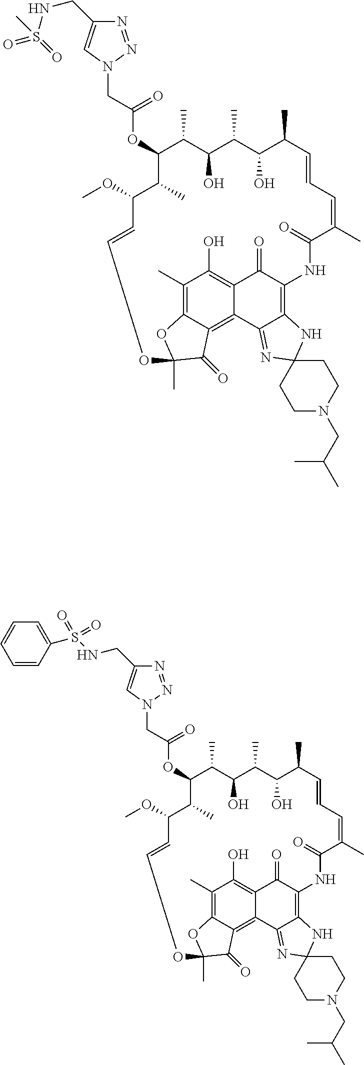 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CNS(C)(=O)=O)nn2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CNS(=O)(=O)c3ccccc3)nn2)[C@@H]1C)C4=O]

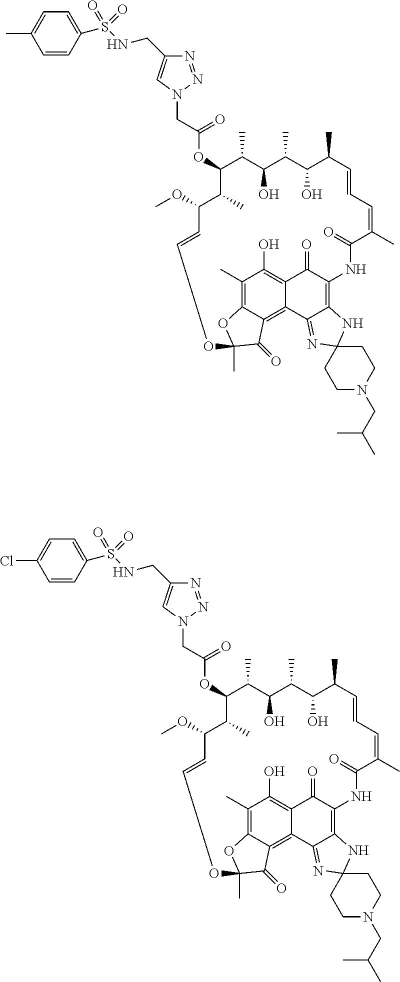

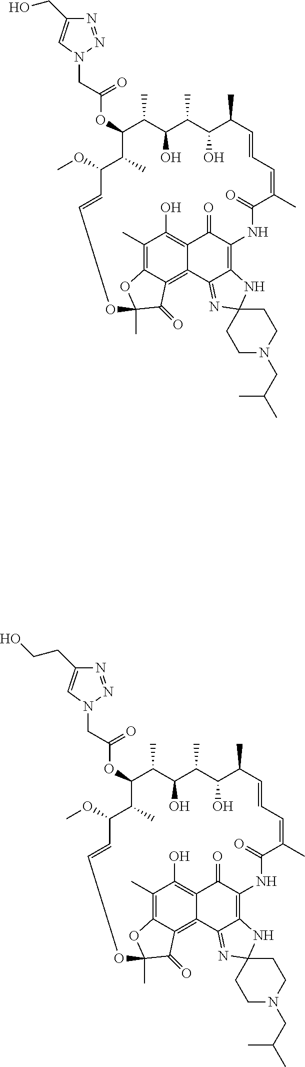 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CO)nn2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CCO)nn2)[C@@H]1C)C4=O]

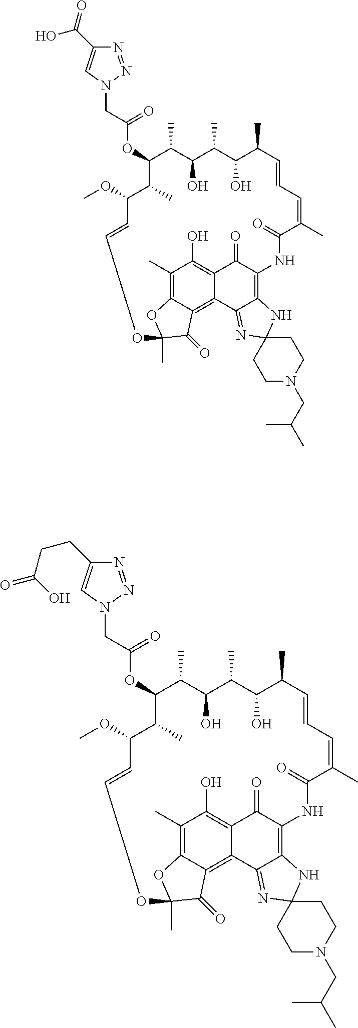 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(C(=O)O)nn2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CCC(=O)O)nn2)[C@@H]1C)C4=O]

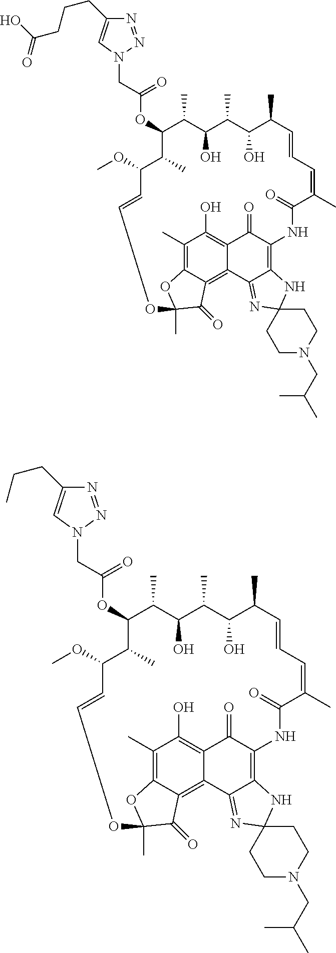

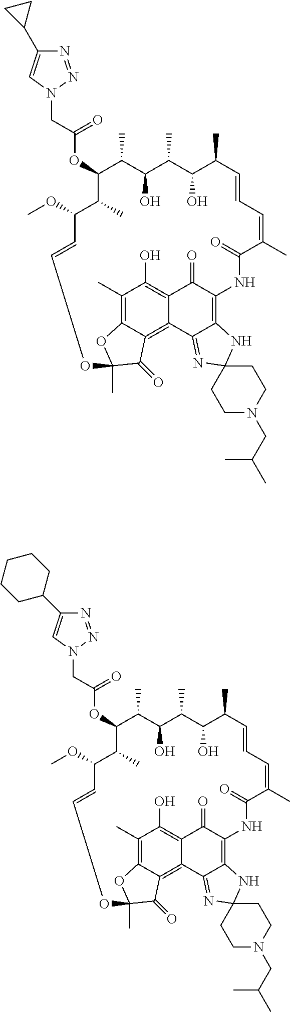 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(C3CC3)nn2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(C3CCCCC3)nn2)[C@@H]1C)C4=O]

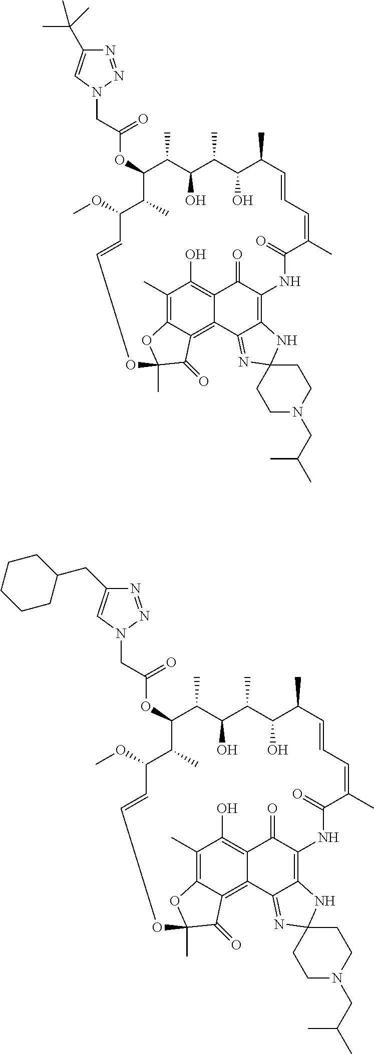 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(C(C)(C)C)nn2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CC3CCCCC3)nn2)[C@@H]1C)C4=O]

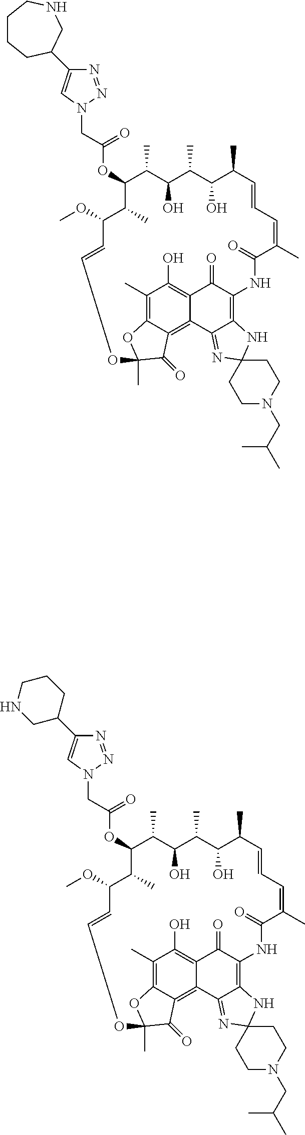 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(C3CCCCNC3)nn2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(C3CCCNC3)nn2)[C@@H]1C)C4=O]

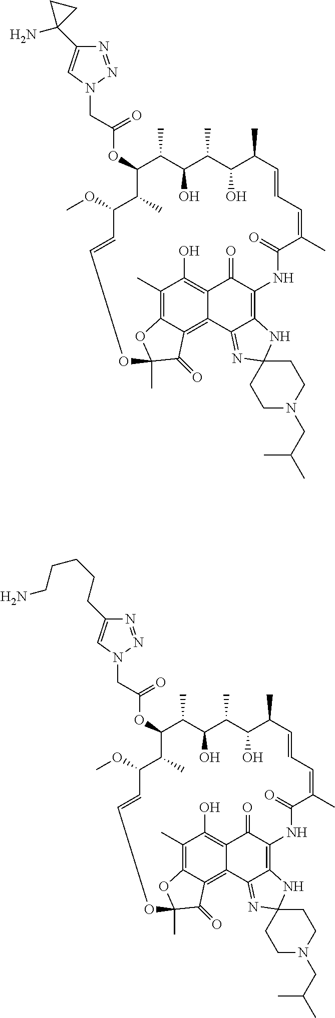 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(C3(N)CC3)nn2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CCCCCN)nn2)[C@@H]1C)C4=O]

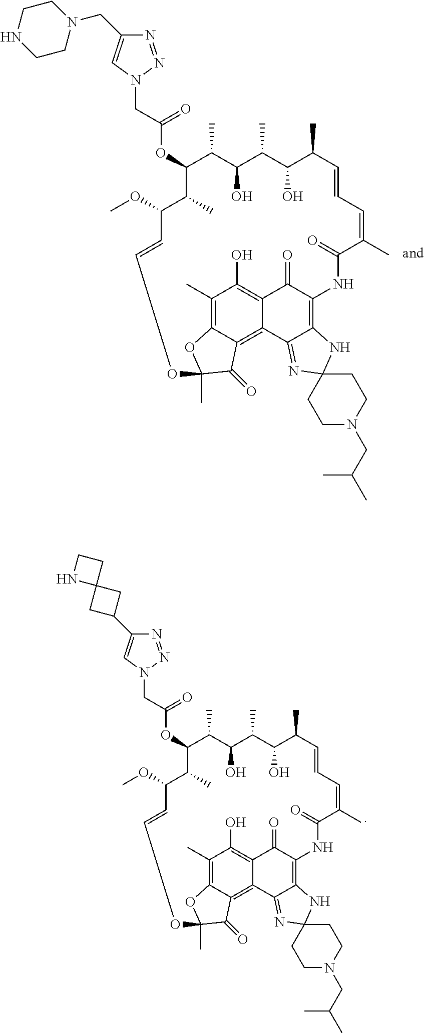 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CN3CCNCC3)nn2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(C3CC5(CCN5)C3)nn2)[C@@H]1C)C4=O]

12. The compound according to claim 1, wherein the compound is selected from the group consisting of:

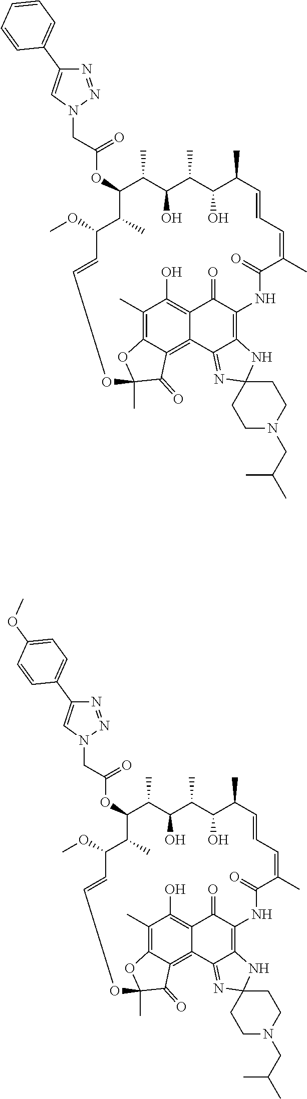 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(-c3ccccc3)nn2)[C@@H]1C)C4=O ; COc1ccc(-c2cn(CC(=O)O[C@H]3[C@H](C)[C@H](O)[C@H](C)[C@@H](O)[C@@H](C)/C=C/C=C(/C)C(=O)NC4=C5NC6(CCN(CC(C)C)CC6)N=C5c5c(c(O)c(C)c6c5C(=O)[C@@](C)(O/C=C/[C@H](OC)[C@H]3C)O6)C4=O)nn2)cc1]

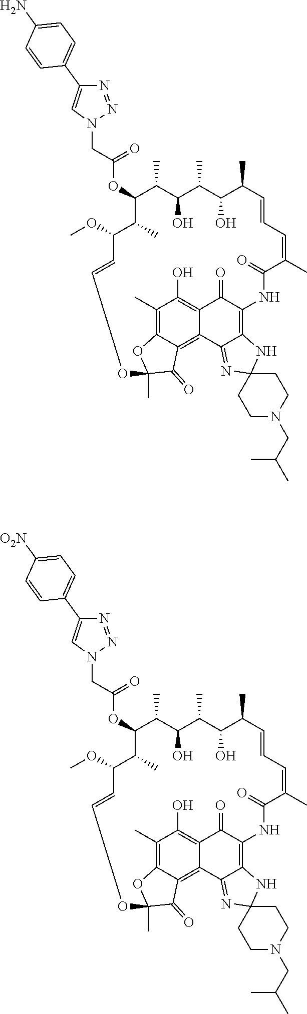 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(-c3ccc(N)cc3)nn2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(-c3ccc([N+](=O)[O-])cc3)nn2)[C@@H]1C)C4=O]

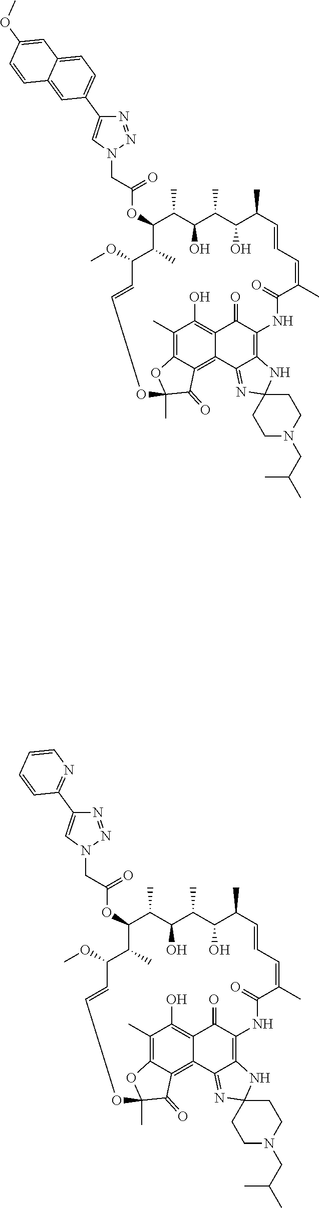 [SMILES: COc1ccc2cc(-c3cn(CC(=O)O[C@H]4[C@H](C)[C@H](O)[C@H](C)[C@@H](O)[C@@H](C)/C=C/C=C(/C)C(=O)NC5=C6NC7(CCN(CC(C)C)CC7)N=C6c6c(c(O)c(C)c7c6C(=O)[C@@](C)(O/C=C/[C@H](OC)[C@H]4C)O7)C5=O)nn3)ccc2c1 ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(-c3ccccn3)nn2)[C@@H]1C)C4=O]

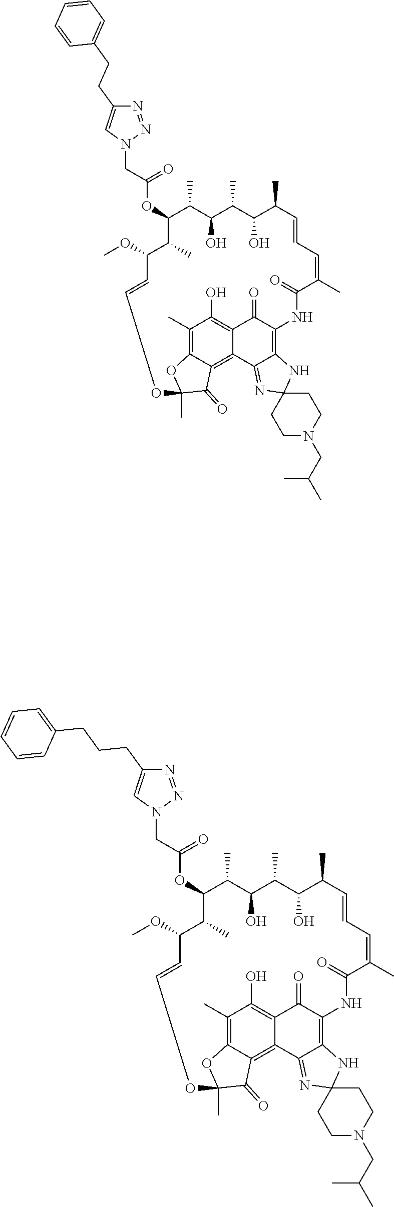 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CCc3ccccc3)nn2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CCCc3ccccc3)nn2)[C@@H]1C)C4=O]

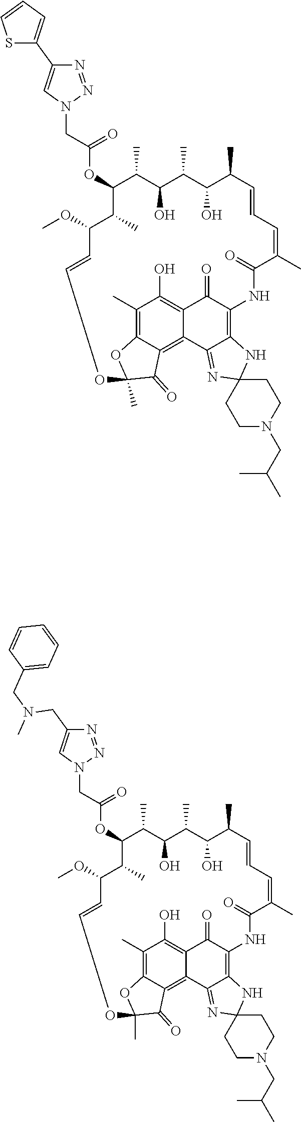 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(-c3cccs3)nn2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CN(C)Cc3ccccc3)nn2)[C@@H]1C)C4=O]

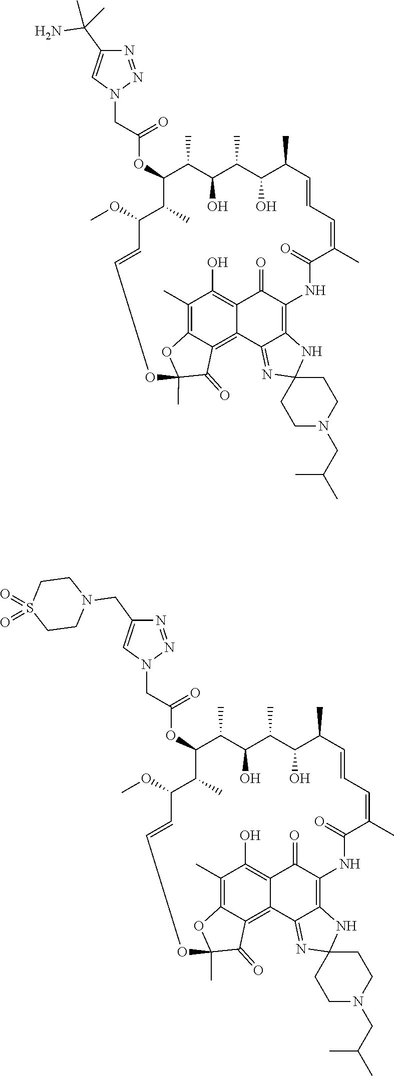 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(C(C)(C)N)nn2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CN3CCS(=O)(=O)CC3)nn2)[C@@H]1C)C4=O]

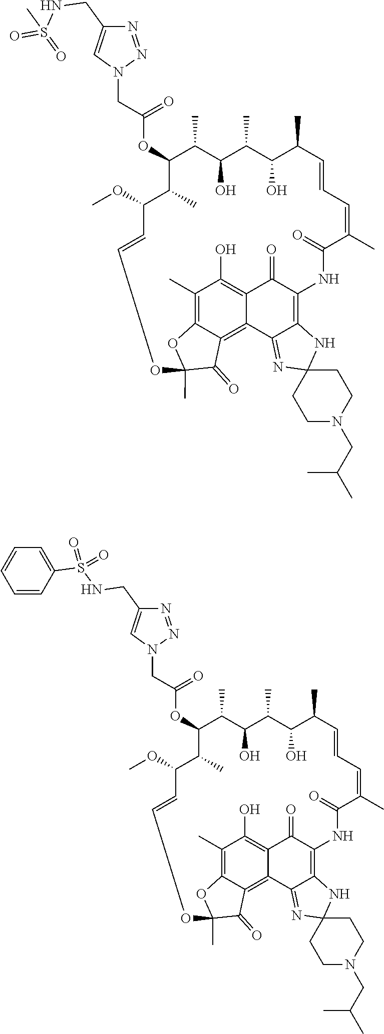 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CNS(C)(=O)=O)nn2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CNS(=O)(=O)c3ccccc3)nn2)[C@@H]1C)C4=O]

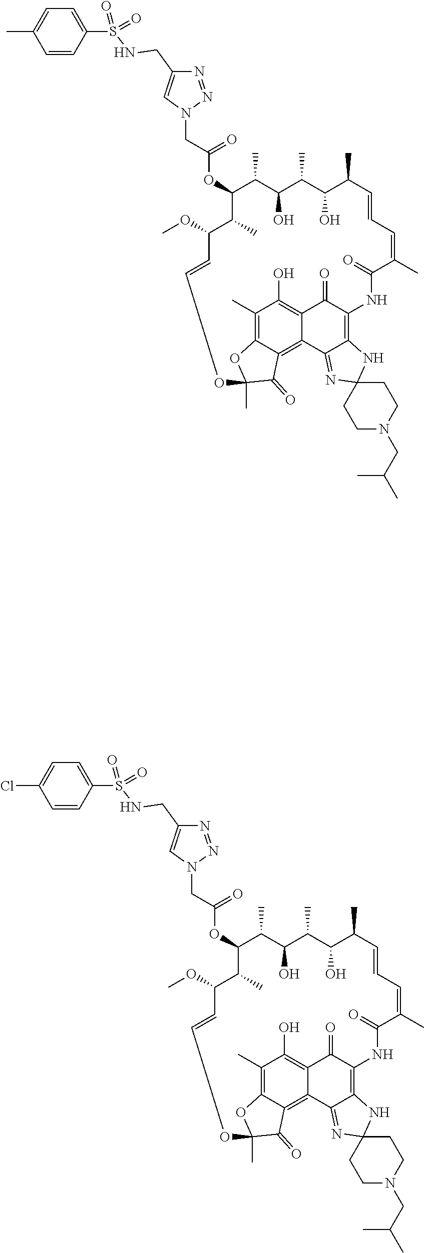

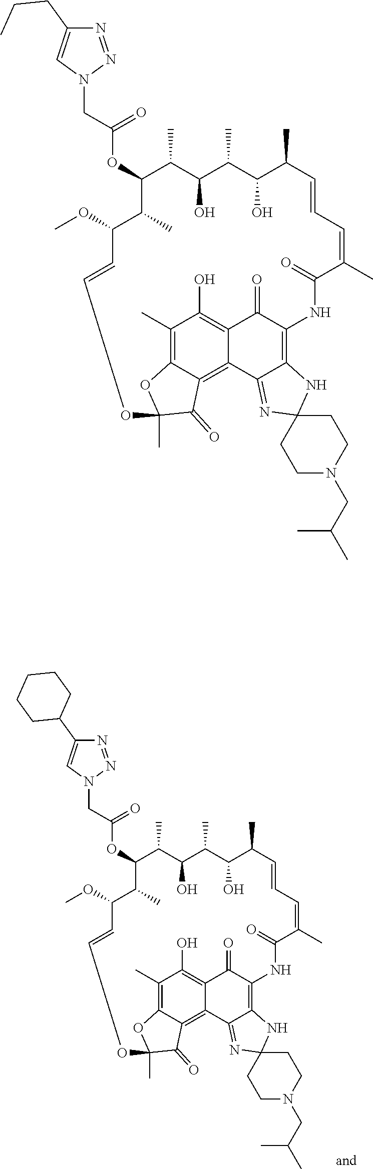 [SMILES: CCCc1cn(CC(=O)O[C@H]2[C@H](C)[C@H](O)[C@H](C)[C@@H](O)[C@@H](C)/C=C/C=C(/C)C(=O)NC3=C4NC5(CCN(CC(C)C)CC5)N=C4c4c(c(O)c(C)c5c4C(=O)[C@@](C)(O/C=C/[C@H](OC)[C@H]2C)O5)C3=O)nn1 ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(C3CCCCC3)nn2)[C@@H]1C)C4=O]

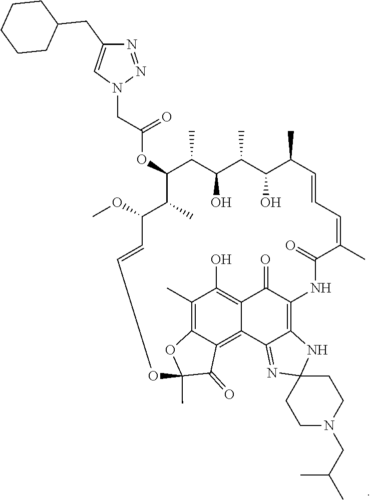 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CC3CCCCC3)nn2)[C@@H]1C)C4=O]

13. The compound according to claim 1, wherein the compound is selected from the group consisting of:

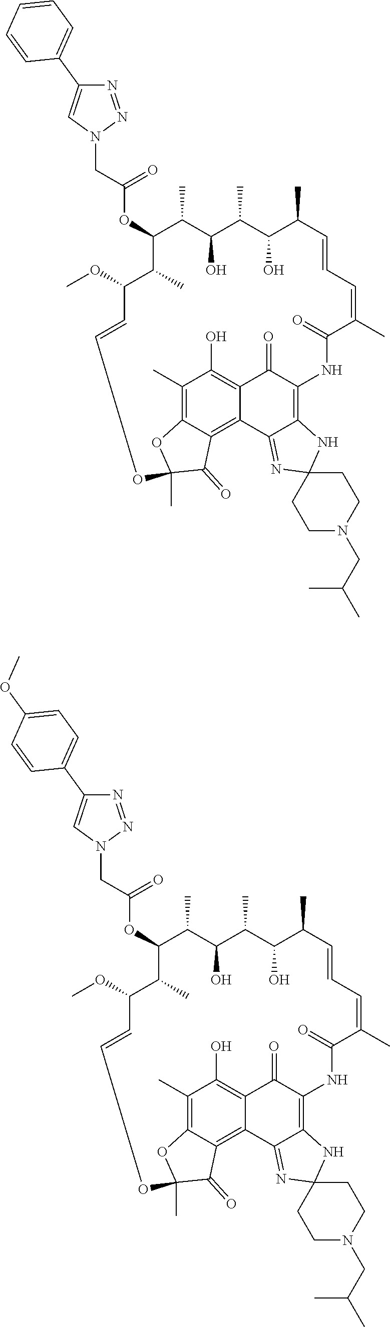 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(-c3ccccc3)nn2)[C@@H]1C)C4=O ; COc1ccc(-c2cn(CC(=O)O[C@H]3[C@H](C)[C@H](O)[C@H](C)[C@@H](O)[C@@H](C)/C=C/C=C(/C)C(=O)NC4=C5NC6(CCN(CC(C)C)CC6)N=C5c5c(c(O)c(C)c6c5C(=O)[C@@](C)(O/C=C/[C@H](OC)[C@H]3C)O6)C4=O)nn2)cc1]

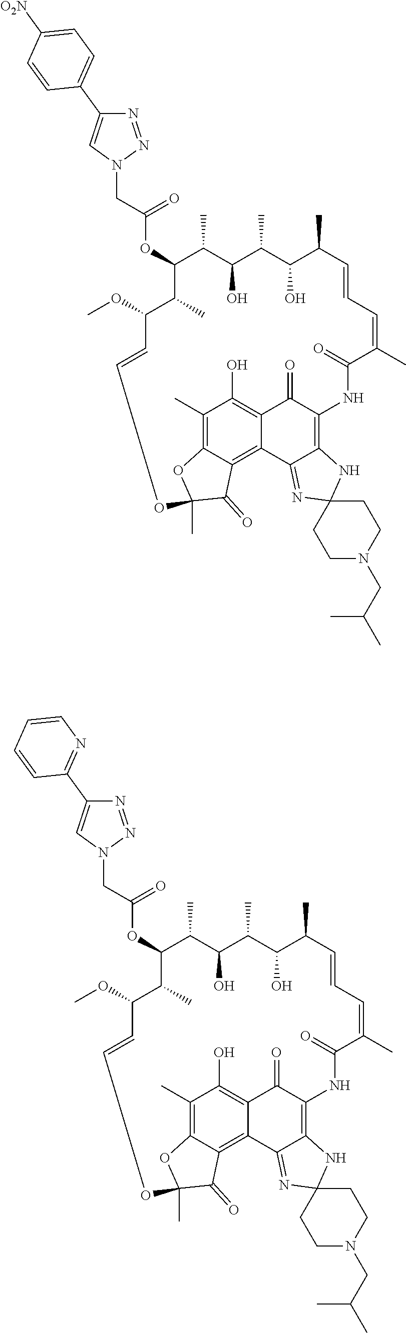 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(-c3ccc([N+](=O)[O-])cc3)nn2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(-c3ccccn3)nn2)[C@@H]1C)C4=O]

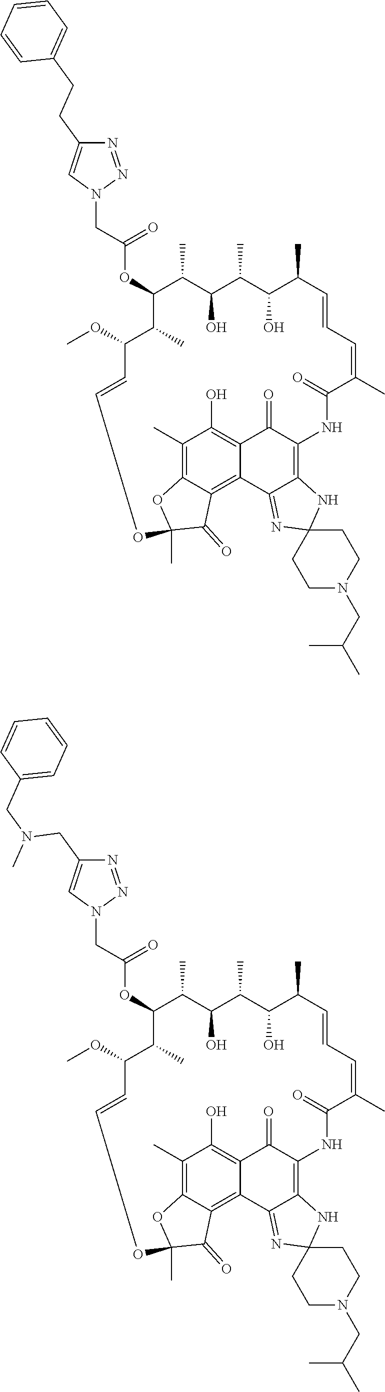 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CCc3ccccc3)nn2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CN(C)Cc3ccccc3)nn2)[C@@H]1C)C4=O]

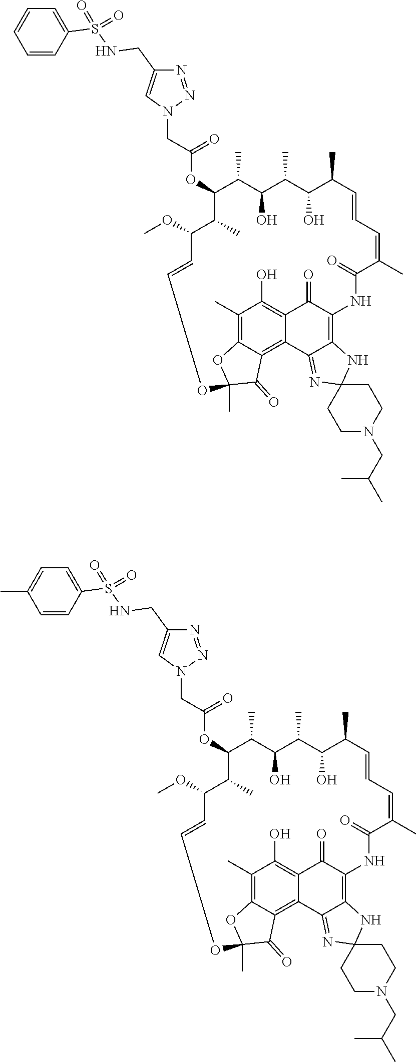

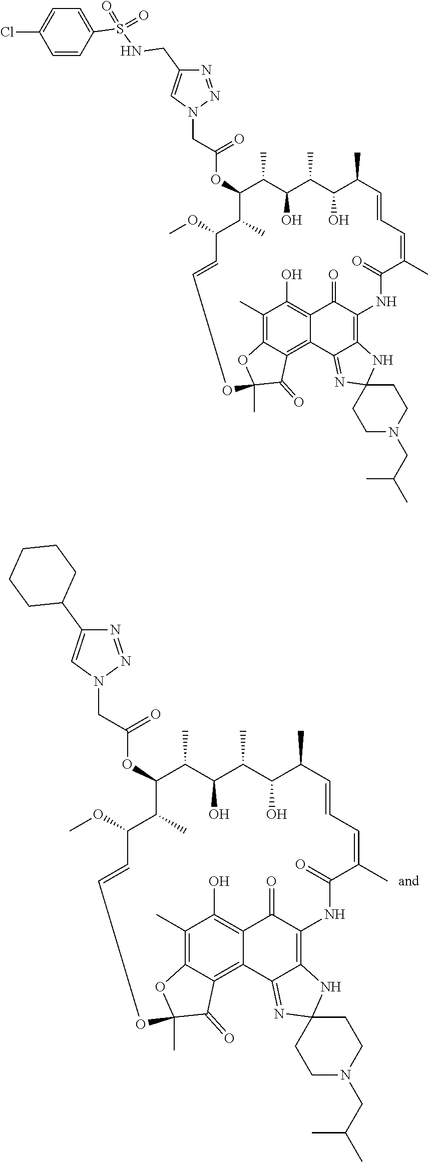 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CNS(=O)(=O)c3ccc(Cl)cc3)nn2)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(C3CCCCC3)nn2)[C@@H]1C)C4=O]

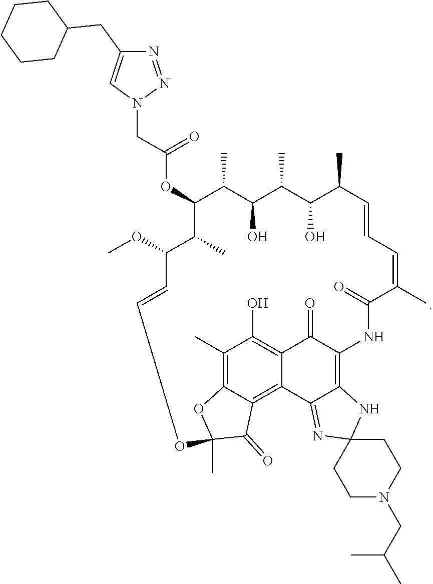 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)Cn2cc(CC3CCCCC3)nn2)[C@@H]1C)C4=O]

14. A pharmaceutical composition comprising at least one compound according to any of the preceding claims, or a pharmaceutically acceptable salt, tautomer, solvate or hydrate thereof, and a pharmaceutically acceptable excipient.

15. A compound according to any of the claims 1 to 13 or a pharmaceutically acceptable salt, tautomer, solvate or hydrate thereof, or a pharmaceutical composition according to claim 14, for use as a medicament.
